SEQUENCE LISTING (33 sequences)

SEQ ID NO: 1 - DNA, 3462 bp, synthetic construct
  source 1..3462: mol_type = other DNA
attgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 2 - DNA, 2817 bp, synthetic construct
  source 1..2817: mol_type = other DNA
attgctccaagatggcggcggcggcggcggcggcagggcagatgacccaaggtgaaaccactcttcttgatcaccctcagcctgaaacctagggaggatgccctggacgccccagctagggtctgacatcaggccccatgcggctcacccgctgctgggctgccctggcagccgccatcatcctcaacctcctagtcttcttctatgtgtcatggctacaacaccagcccagaaactcccgggcccggggtccccgccggacttctgccattggcccccgagtcaccgtcctgattcgggaatttgaggcttttgacaacgcggtgccagagctagtggattcctttttgcagcaggacccagcccagcccgtggtagtggcggccgacacactcccttacccacccctggccttgcctcgcatccccaacgttcgcctggctctgctccagccagccctggaccggccagcggcggcctcgcgcccggagacctacgtagccaccgagtttgtggctctagtgcctgatggagcgcgggccgagtcaccaggccacctggagcgaatggtggaggcgctccgagggagcagcgcgcgcctagtggccgccccggtcgccaccgccaacccagcgcggtgcctagctctgaacgtcagcctgcgggagtggactgcgcgctacgacccagctcccagcgcgccccgctgcgacgctttggatggcgacgctgtgctgctgatgcgctcccgcgacctcttcaacctctcggtgcccctggcgcggccgctggccaccagcctcttcctacagaccgccctgcgcggctgggcagtgcagctgctggacttgaccttcgccgcggcgcgccagccaccgctggccaccgcccacgcgcgctggaaggcggaacgtgaggggcgctcacggagagcggcgctgctgcgctcgttgggaatccgtctggtgagctgggaaggcgggcggctagagtggtttggctgcagcaaggagagcgcgcgctgcttcggtacggtggcgggcgacacacccgcctacctgtatgagggccgctggaccccaccttgttgcctgcgcgcgctgcgcgagactgcgcgctacgtggtgggcgtgctggaggcggcgggcgtgcgctactggttggagggcggctcgctgctgggtgcagctcgccacggcgacatcatcccttgggactacgacgtagatctgggcatctacctggaggacgtgggcaactgcgagcagttgcggggtgccgaagctggctcggtagtggatgaacgcggctttgtgtgggagaaggcggtggagggcgacttcttccgagtacagtacagtgagaacaaccacctgcacgtggacctgtggcccttttacccccgcaatggggttatgaccaaggacacgtggctggaccaccggcaggatgttgagttcccagagcacttcctgcagccacttgtccccctgccctttgcgggtttcatggcacaggcccctaacaactaccgccgcttcctggagctgaagtttgggcctggggtcatcgagaacccggagtaccccaaccccgcactcttaagcttgacaggcggttgaagccctggtacccacatctggggctaggtgaacagcagggtacaagagtctgttcttgcctcggattgtgtatggatctgaaccagtttgtgattgggtagtggggctgtgcactggagggaagaaaaaaaaagggtgaactggccaaggaagattggagggaacacacaggacagtttcagctttggcgggaagcagaacccaaagactggagcaactcagcctcatgagggaggaaagtggtggcctttgatagaaaagcccagggagtctttagggctctgcatagtctctgccctatattccaagagctgtaggcccttggagagtgagatccggagaacagacgagccagcctcagtctaccacatacaacttaggtatccctggaccagttagttacccagtctgtttcatttgagataccgaatgcctccctttgtgtttaggatcctcaggacgcttggctccagtgcttgcttcctccactagagggcacttctgtcccgcttcagttcgtgtttgatgaccgctcgcggctctgcttcatcaccttcatctctggttttcttggagtggctctgggaggtgggacagcactggccacctcctctcctccccgaatgtggcactttcctttgagagtgctgcttgatgtatctgctgcttctaaccccatcttgctcttggaggactgactgggctctctctggtttgggatctggccaatgcattacctctttatttcagccctctgcacctgttccccgccttccccctcccttctaggaagggataaaccttcgccatagctcacatttacaaagagctttcgggctcaggcatacactcggaagtgacagctgctattacgtccattttccggggaggaaaactgaggccaaaatgacagtcacttgccagtaatcagatattttaggaagtggctagtaagggacttgaattcagatcaccgtttcaaggcccacacttcatttcctctctaggttctgctgtagaaatagtacccaagtggagccttgccatggctgtgccactcaaacaggtttaaaaaaaaattctcatttaattatgcatataaattatgttcaaaccagtctgtatattgttaaataagacttaaattatgctactttaaagtttaaaaattcaagacaaatagaaaaaaaaaaaa

SEQ ID NO: 3 - DNA, 134 bp, synthetic construct
  source 1..134: mol_type = other DNA
attgctccaagatggcggcggcggcggcggcggggcagatgacccaaggtgaaaccactcttcttgatcaccctcagcctgaaacctagggaggatgccctggacgccccagctagggtctgacatcaggcccc

SEQ ID NO: 4 - DNA, 1195 bp, synthetic construct
  source 1..1195: mol_type = other DNA
agccctggtacccacatctggggctaggtgaacagcagggtacaagagtctgttcttgcctcggattgtgtatggatctgaaccagtttgtgattgggtagtggggctgtgcactggagggaagaaaaaaaaagggtgaactggccaaggaagattggagggaacacacaggacagtttcagctttggcgggaagcagaacccaaagactggagcaactcagcctcatgagggaggaaagtggtggcctttgatagaaaagcccagggagtctttagggctctgcatagtctctgccctatattccaagagctgtaggcccttggagagtgagatccggagaacagacgagccagcctcagtctaccacatacaacttaggtatccctggaccagttagttacccagtctgtttcatttgagataccgaatgcctccctttgtgtttaggatcctcaggacgcttggctccagtgcttgcttcctccactagagggcacttctgtcccgcttcagttcgtgtttgatgaccgctcgcggctctgcttcatcaccttcatctctggttttcttggagtggctctgggaggtgggacagcactggccacctcctctcctccccgaatgtggcactttcctttgagagtgctgcttgatgtatctgctgcttctaaccccatcttgctcttggaggactgactgggctctctctggtttgggatctggccaatgcattacctctttatttcagccctctgcacctgttccccgccttccccctcccttctaggaagggataaaccttcgccatagctcacatttacaaagagctttcgggctcaggcatacactcggaagtgacagctgctattacgtccattttccggggaggaaaactgaggccaaaatgacagtcacttgccagtaatcagatattttaggaagtggctagtaagggacttgaattcagatcaccgtttcaaggcccacacttcatttcctctctaggttctgctgtagaaatagtacccaagtggagccttgccatggctgtgccactcaaacaggtttaaaaaaaaattctcatttaattatgcatataaattatgttcaaaccagtctgtatattgttaaataagacttaaattatgctactttaaagtttaaaaattcaagacaaatagaaaaaaaaaaaa

SEQ ID NO: 5 - skipped

SEQ ID NO: 6 - DNA, 365 bp, synthetic construct
  source 1..365: mol_type = other DNA
attgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttctccaccatgcggctcacccgctgccaggctgccctggcggccgccatcaccctcaaccttctggtcctcttctatgtctcgtggct

SEQ ID NO: 7 - DNA, 366 bp, synthetic construct
  source 1..366: mol_type = other DNA
agactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatgcggctcacccgctgccaggctgccctggcggccgccatcaccctcaaccttctggtcctcttctatgtctcgtggct

SEQ ID NO: 8 - DNA, 436 bp, synthetic construct
  source 1..436: mol_type = other DNA
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 9 - DNA, 32 bp, synthetic construct
  source 1..32: mol_type = other DNA
attgctccaagatggcggcggcggcggcagcg

SEQ ID NO: 10 - DNA, 42 bp, synthetic construct
  source 1..42: mol_type = other DNA
tgtacaattgctccaagatggcggcggcggcggcggcggcag

SEQ ID NO: 11 - RNA, 3463 nt, Homo sapiens
  source 1..3463: mol_type = mRNA
cattgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 12 - RNA, 3411 nt, Mus musculus
  source 1..3411: mol_type = mRNA
cattgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 13 - RNA, 3621 nt, Homo sapiens
  source 1..3621: mol_type = mRNA
gggaaaggtgggctgctacaagcaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 14 - RNA, 3702 nt, Homo sapiens
  source 1..3702: mol_type = mRNA
cattgctccaagatggcggcggcggcggcagcggaggagatgctctgctgaggggcagttgctatggttacagggcctggaccttcctccggaaggtgagaaacaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 15 - RNA, 3535 nt, Homo sapiens
  source 1..3535: mol_type = mRNA
cattgctccaagatggcggcggcggcggcagcggaggagatgctctgctgaggggcagttgctatggttacagggcctggaccttcctccggaaggtgagaaacaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 16 - RNA, 3483 nt, Homo sapiens
  source 1..3483: mol_type = mRNA
cattgctccaagatggcggcggcggcggcagcggaggagatgctctgctgaggggcagttgctatggttacagggcctggaccttcctccggaaggtgagaaacaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 17 - RNA, 3332 nt, Homo sapiens
  source 1..3332: mol_type = mRNA
cattgctccaagatggcggcggcggcggcagcggaggagatgctctgctgaggggcagttgctatggttacagggcctggaccttcctccggaaggtgagaaacaggagcgcagctcagctgggctggaactgccctcctggaactcccccagcctacaacctaggaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 18 - RNA, 3524 nt, Homo sapiens
  source 1..3524: mol_type = mRNA
cattgctccaagatggcggcggcggcggcagcggaggagatgctctgctgaggggcagttgctatggttacagggcctggaccttcctccggaaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 19 - RNA, 3472 nt, Homo sapiens
  source 1..3472: mol_type = mRNA
cattgctccaagatggcggcggcggcggcagcggaggagatgctctgctgaggggcagttgctatggttacagggcctggaccttcctccggaaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 20 - RNA, 3321 nt, Homo sapiens
  source 1..3321: mol_type = mRNA
cattgctccaagatggcggcggcggcggcagcggaggagatgctctgctgaggggcagttgctatggttacagggcctggaccttcctccggaaggagcgcagctcagctgggctggaactgccctcctggaactcccccagcctacaacctaggaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 21 - RNA, 3629 nt, Homo sapiens
  source 1..3629: mol_type = mRNA
cattgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 22 - RNA, 3260 nt, Homo sapiens
  source 1..3260: mol_type = mRNA
cattgctccaagatggcggcggcggcggcagcgggagcgcagctcagctgggctggaactgccctcctggaactcccccagcctacaacctaggaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 23 - RNA, 3442 nt, Homo sapiens
  source 1..3442: mol_type = mRNA
ggggacagtaggaaggggggccggcggcgagcgcgagcctcgcccacccgggtctacaccgaagggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 24 - RNA, 3275 nt, Homo sapiens
  source 1..3275: mol_type = mRNA
ggggccggcggcgagcgcgagcctcgcccacccgggtctacaccgaagggagcgcagctcagctgggctggaactgccctcctggaactcccccagcctacaacctaggaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 25 - RNA, 3621 nt, Homo sapiens
  source 1..3621: mol_type = mRNA
cgcccacccgggtctacaccgaagggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 26 - RNA, 3900 nt, Homo sapiens
  source 1..3900: mol_type = mRNA
gggggccggcgggtgggagatcgcgccccgaggagggcgagggagacccaggccccgcgcgacggtccccctcgctggagtgagactgaggaaggggccgctttctgcccccctcctcccacttcgccccagagaaagagccttaccccttccacacccaaacaccactctcctggacaattacttttactctgagagaaagggcgaccagcagtggccccatcctctctgattgccggccagaggagatgctctgctgaggggcagttgctatggttacagggcctggaccttcctccggaaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 27 - RNA, 3839 nt, Homo sapiens
  source 1..3839: mol_type = mRNA
gggggccggcgggtgggagatcgcgccccgaggagggcgagggagacccaggccccgcgcgacggtccccctcgctggagtgagactgaggaaggggccgctttctgcccccctcctcccacttcgccccagagaaagagccttaccccttccacacccaaacaccactctcctggacaattacttttactctgagagaaagggcgaccagcagtggccccatcctctctgattgccggccaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgtaagttgcatggcttttgtccgtcttttttttgtttgtttgtttgttttgagacagggtctcacccaggctagagtgaagtggagcaatctcggctcactgcaacctccgcctcctgagttcaagtgattctcacacctcagcctccccaatagctgggattacaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 28 - RNA, 3816 nt, Homo sapiens
  source 1..3816: mol_type = mRNA
tgtcatggtaacccttccccagcggtgggggcgaggtggtggggagcagccttcccctcgcttccacctttgtggttatggagccccaaacctcttaggactctgcaccaggagaaggttgctaaggtaacctaagtgtccgtggtggtccttctgctcctatcctcattgattactatggcaacctcccccccacctagcattcaggtagaaaggaccccagccccaaggacactgatctctaagagaagcacctcttccccagcccctcccccatagccacctagttggcattaccatggtaaccgtcttccccttcccatcgtgggaggcccatgcaccaaactaaccaggtgtttttatttagaggagatgctctgctgaggggcagttgctatggttacagggcctggaccttcctccggaaggtgagaaacaggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggatgccccggaggcccagctagccccagacttcggccccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgaagccctgataacctcgcctttgtttttcgggggtctgtctggatgtggagaagctctgtgtgagcggtgaggggtggagggatgtcgcggagaggggaagggggaaactgaccaagaaagaaattctaaggagagcatgagagaaggctggcattggcaggaggagagcaccaggacgaggatgggaagcgacctccagatttatcaaatggtcatgcccactgggagccgtggatatgcgtggggacatcctgggtcatctcagtcatggagggagacggggatgtcacgccgtcccgcagggcccagcacagccccagacccgaaaaaagtgttctgcccaagattccgagagccctgcgctctagggcaggggcagagttttggaaacagtgcaggctctggagccagactggcgagattcaaatcctggctctatcgcttcggagccaggtgggcctgggggggcgtcgcagtctctctgtgcctcagttgcttccaggatgcgggacccttggctgcaggggttgcttccgccactagagggcgcgccggtcccgctcctggtggcccactgtggctgcccgggcgacagtacgcccagggcctgtgttccatagccatctactctcttgagcctttggacttctctccaagcccctgtgggaggcggacagcagtgaccacctccccttcttttggactgcgacctccttccctcctgggagagccctgtgacctgcatgctactcttaactgttctattcaagactgaatagaagtatttcagtcttgcagaggaggaaatgctcagagctccgaggtgcggctgtggtcgagaaccgggtgctgggccgggcgcgggggctcacgcctgtaatcccagcactttgggaggccgaggtgggaggatcgcttgagcccaggagtctgagaccagcctcggcaacatgccaagaccccgtctctatttttaaaaaagaaaaagaaccgacttctgaatcgcagctccactcatgactaatacctcattatttcagctgtctgcacctaattccccacttgcacggcagtgtagacaataaccatagctcacactcactgagcacctactgggtaccaggcaccattctcagtgtttcacctggatcaactaatgcgtccctcacctcagccctctgaagtgacagctgctattattttcattacacagatgaaaaagctgaggccagaatcgtgaagtcacttgctcaaggtcaggcagcttaggaaggggcagatcgggggcttgaacccaggtggtcaggctctggagcccacaattgtcttacccactatgcccctctctagtcatggtccccaagaggggcttggagacccacttagcaggtgaaagcaatggcagccttccttatttgattatgcacctaagaataaatggtatttgggcatgtattcccaatatgtgtatatttatttataaatatatacagatactattatctgtatgttagtaataaagcttaaattattccattttaaaattatgaatatgaatagggttttttttatgtttcttgcctcatcccaatgacttttgcacacccaggtgtgagcacccagcattcaagaccacg

SEQ ID NO: 29 - DNA, 133 bp, synthetic construct
  source 1..133: mol_type = other DNA
agctcagctgggctggaactgccctcctggaactcccccagcctacaacctaggaggtgcagggactgaggctcaggccaaatcgcaactcagacccagtgaacccaaggcctgaagagaatttggattcatt

SEQ ID NO: 30 - DNA, 21 bp, synthetic construct
  source 1..21: mol_type = other DNA
ttgttaacatgcggctcaccc

SEQ ID NO: 31 - DNA, 20 bp, synthetic construct
  source 1..20: mol_type = other DNA
taccggttcaaccgcctgtc

SEQ ID NO: 32 - protein, 26 aa, synthetic construct
APNNYRRFLELKFGPGVIENPQYPNP

SEQ ID NO: 33 - DNA, 401 bp, synthetic construct
  source 1..401: mol_type = other DNA
attgctccaagatggcggcggcggcggcagcgggagcgcagctcagctgggctggaactgccctcctggaactcccccagcctacaacctaggaggtgcagggactgaggctcaggccaaatcgcaactcagacccagtgaacccaaggcctgaagagaatttggattcatttaccttgttttgtggggactggagagacaagtaaactctcagagtaactgtcccctctgactaccatttctaaggcaagccccctgtttctactcttgcgccccctgctggtttcctgccctgtctgatgccccggaggcccagctagccccagacttcggccccatgcggctcacccgctgccaggctgccctggcggccgccatcaccctcaaccttctggtcctct

(19) United States
(12) Patent Application Publication
(10) Pub. No.: US 2025/0312398 A1
(43) Pub. Date: Oct. 9, 2025

(54) METHOD FOR EXPRESSING A MUSCLE-SPECIFIC GENE AND CASSETTES FOR SAME

(71) Applicant: UNIVERSITY OF WASHINGTON, Seattle, WA (US)

(72) Inventors: Jeffrey Scott CHAMBERLAIN, Seattle, WA (US); Stephen D. HAUSCHKA, Seattle, WA (US); Halli Claire BENASUTTI, Seattle, WA (US)

(73) Assignee: UNIVERSITY OF WASHINGTON, Seattle, WA (US)

(21) Appl. No.: 18/860,319

(22) PCT Filed: Apr. 27, 2023
(86) PCT No.: PCT/US2023/066296

§ 371 Date: Oct. 25, 2024

Related U.S. Application Data
(60) Provisional application No. 63/336,678, filed on Apr. 29, 2022.

(51) Int. Cl. A61K 35/76; A61K 38/45; A61K 48/00; C07K 14/47; C12N 9/12; C12N 15/86

(52) U.S. Cl. CPC A61K 35/76; A61K 38/45; A61K 48/0058; A61K 48/0066; A61K 48/0075; C07K 14/4707; C12N 9/1288; C12N 15/86; C12N 2750/14122; C12N 2750/14143; C12N 2750/14145; C12N 2830/008; C12Y 207/08

(57) ABSTRACT

The invention described here contains a nucleic acid expression cassette comprising a transcriptional regulatory region operably linked to a nucleic acid sequence encoding a FKRP, an RNA transcript comprising a modified 5′ and/or 3′ untranslated region (UTR) that will be utilized to treat a variety of FKRP-mediated diseases.

### CROSS REFERENCE TO RELATED APPLICATIONS

This application claims benefit under 35 U.S.C. § 119(e) to U.S. Provisional Application No. 63/336,678 filed Apr. 29, 2022, the contents of which is incorporated herein by reference in its entirety.

### SEQUENCE LISTING

The instant application contains a Sequence Listing that has been submitted in XML format via Patent Center and is hereby incorporated by reference in its entirety. Said XML copy, created on Apr. 26, 2023, is named “034186-191920WOPT_SL.xml” and is 101,526 bytes in size.

### FIELD OF THE INVENTION

The field of invention relates to gene therapy for the treatment of FKRP-mediated diseases.

### BACKGROUND

Neuromuscular disorders can result from genetic mutations in key genes that regulate optimal muscular production. One gene important for optimal muscular production is fukutin-related protein (FKRP). It is a glycosyl-transferase localized within the trans-golgi complex and mediates glycosylation which is required for the maturation and function of α-dystroglycan, an essential component of the dystrophin-glycoprotein complex (DGC). Currently, there are no significant treatment options for patients with FKRP disorders. One type of treatment that is being utilized to combat genetic muscle diseases is gene therapy, which must be adapted for each individual type of dystrophy.

### SUMMARY OF THE INVENTION

The methods and compositions described herein are based, in part, on the discovery that muscle expression of FKRP can be improved by (i) using a muscle-specific expression cassette (MSEC) to deliver FKRP to a cell, and (ii) modifying the nucleic acid encoding FKRP such that the resulting transcript comprises a modification to the 5′ and/or 3′ untranslated region (UTR). Such modifications to the 5′ and/or 3′ UTR can increase the expression levels of FKRP as compared to a construct comprising a wild-type 5′ and/or 3′ UTR. The methods and compositions provided herein can be used to express FKRP in a cell or subject for the treatment of FKRP-mediated diseases or disorders including, but not limited to, limb girdle muscular dystrophy type 2I/R9 (LGMD2i, also known as LGMDR9), Walker-Warburg syndrome, or muscle-eye-brain disease (MED).

One aspect provided herein describes a nucleic acid expression cassette comprising a transcriptional regulatory region operably linked to a nucleic acid sequence encoding a fukutin-related protein (FKRP) RNA transcript that comprises a modified 5′ and/or 3′ untranslated region (UTR).

In one embodiment of any of the aspects described herein, the modified 5′ untranslated region (UTR) is truncated as compared to the 5′ UTR of wild-type FKRP.

In one embodiment of any of the aspects described herein, the modified 5′ UTR comprises a deletion of at least one nucleotide, a plurality of nucleotides or all of the nucleotides in the 5′ UTR region.

In one embodiment of any of the aspects described herein, the modification of the 5′UTR comprises deletion or disruption of a G-quadruplex, or a hairpin in the 5′ UTR.

In one embodiment of any of the aspects described herein, the modification comprises a modification to the Kozak consensus sequence.

In one embodiment of any of the aspects described herein, the modified 3′ UTR is truncated compared to the 3′ UTR of wild-type FKRP.

In one embodiment of any of the aspects described herein, the modification to the 3′ UTR comprises deletion of at least one nucleotide, a plurality of nucleotides or all of the nucleotides in the 3′ UTR region.

In one embodiment of any of the aspects described herein, the nucleic acid encoding FKRP comprises a modification in each of the 5′ and 3′ UTRs.

In one embodiment of any of the aspects described herein, the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

In one embodiment of any of the aspects described herein, the transcriptional regulatory region comprises a muscle-specific expression cassette (MSEC).

In one embodiment of any of the aspects described herein, the MSEC is selected from the group consisting of CK8e.

In one embodiment of any of the aspects described herein, upon administration to a cell, expression level of an FKRP mRNA or protein is higher when operably linked to an MSEC than the expression level of the FKRP mRNA or protein when operably linked to a CK8e transcriptional regulatory region.

In one embodiment of any of the aspects described herein, upon administration to a cell, expression level of an FKRP mRNA or protein is lower when operably linked to an MSEC than the expression level of the FKRP mRNA or protein when operably linked to a CK8e transcriptional regulatory region.

Another aspect provided herein relates to an RNA transcript generated by transcription of the nucleic acid expression cassette of any one of the embodiments described herein.

Also provided herein, in another aspect, is an adeno-associated viral vector (AAV) comprising the nucleic acid expression cassette of any one of the embodiments described herein.

In one embodiment of any of the aspects described herein, the adeno-associated viral vector is selected from the group consisting of: an AAVRh74 vector, an AAV8 vector, an AAV9 vector, an AAV6 vector, an AAV7 vector, an AAV218 vector, a NP vector, a NP 66 vector, a NP 22 vector, an AAVpo. 1 vector, a MyoAAV vector, and an AAVMyo vector.

In one embodiment of any of the aspects described herein, the adeno-associated viral vector comprises an internal terminal repeat (ITR), a muscle-specific cassette, a nucleic acid specific to FKRP, a polyadenylation signal (pA+), and/or a second ITR.

Another aspect provided herein describes an engineered cell comprising or expressing a nucleic acid expression cassette of any one of the embodiments described herein.

In one embodiment of any of the aspects described herein, the modified 5′ untranslated region (UTR) is truncated as compared to the wild-type 5′ UTR of FKRP.

In one embodiment of any of the aspects described herein, the modified 5′ UTR comprises a deletion of at least one nucleotide, a plurality of or all of the nucleotides in the 5′ UTR region.

In one embodiment of any of the aspects described herein, the modification of the 5′UTR comprises deletion or disruption of a G-quadruplex, or a hairpin in the 5′ UTR.

In one embodiment of any of the aspects described herein, the modification comprises a modification to the Kozak consensus sequence.

In one embodiment of any of the aspects described herein, the modified 3′ UTR is truncated compared to the 3′UTR of wild-type FKRP.

In one embodiment of any of the aspects described herein, the modification to the 3′ UTR comprises deletion of at least one nucleotide, a plurality of nucleotides or all of the nucleotides in the 3′ UTR region.

In one embodiment of any of the aspects described herein, the nucleic acid encoding FKRP comprises a modification in each of the 5′ and 3′ UTRs.

In one embodiment of any of the aspects described herein, the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

Another aspect provided herein relates to a method of expressing an FKRP gene product in a subject comprising administering an adeno-associated viral vector according to any one of the embodiments described herein to a subject in need thereof.

In one embodiment of any of the aspects described herein, the FKRP gene product is a RNA transcript and/or a protein.

In one embodiment of any of the aspects described herein, the subject in need thereof comprises limb girdle muscular dystrophy type 21/R9 (LGMD2i), Walker-Warburg syndrome, or muscle-eye-brain disease (MED).

In one embodiment of any of the aspects described herein, the modified 5′ untranslated region (UTR) is truncated as compared to the 5′ UTR of wild-type FKRP.

In one embodiment of any of the aspects described herein, the modified 5′ UTR comprises a deletion of at least one nucleotide, a plurality of nucleotides or all of the nucleotides in the 5′ UTR region.

In one embodiment of any of the aspects described herein, the modification of the 5′UTR comprises deletion or disruption of a G-quadruplex, or a hairpin in the 5′ UTR.

In one embodiment of any of the aspects described herein, the modification comprises a modification to the Kozak consensus sequence.

In one embodiment of any of the aspects described herein, the modified 3′ UTR is truncated compared to the 3′ UTR of a wild-type FKRP.

In one embodiment of any of the aspects described herein, the modification to the 3′ UTR comprises deletion of at least one nucleotide, a plurality of nucleotides or all of the nucleotides in the 3′ UTR region.

In one embodiment of any of the aspects described herein, the nucleic acid encoding FKRP comprises a modification in each of the 5′ and 3′ UTRs.

In one embodiment of any of the aspects described herein, the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

In one embodiment of any of the aspects described herein, the administration of the AAV vector comprises intravenous and/or intramuscular injection.

In one embodiment of any of the aspects described herein, the subject is a human.

Also provided herein, in another aspect, is a method for reducing at least one symptom of an FKRP-mediated disease or disorder, the method comprising administering an AAV vector of any one the embodiments to a subject in need thereof, thereby reducing at least one symptom of an FKRP-mediated disorder.

In one embodiment of any of the aspects described herein, the FKRP-mediated disease or disorder comprises limb girdle muscular dystrophy type 21/R9 (LGMD2i), Walker-Warburg syndrome, or muscle-eye-brain disease (MED).

In one embodiment of any of the aspects described herein, the modified 5′ untranslated region (UTR) is truncated as compared to the wild-type 5′ UTR of FKRP.

In one embodiment of any of the aspects described herein, the modified 5′ UTR comprises a deletion of at least one nucleotide, a plurality of nucleotides or all of the nucleotides in the 5′ UTR region.

In one embodiment of any of the aspects described herein, the modification of the 5′UTR comprises deletion or disruption of a G-quadruplex, or a hairpin in the 5′ UTR.

In one embodiment of any of the aspects described herein, the modification comprises a modification to the Kozak consensus sequence.

In one embodiment of any of the aspects described herein, the modified 3′ UTR is truncated compared to the 3′ UTR of a wild-type FKRP.

In one embodiment of any of the aspects described herein, the modification to the 3′ UTR comprises deletion of at least one nucleotide, a plurality of nucleotides or all of the nucleotides in the 3′ UTR region.

In one embodiment of any of the aspects described herein, the nucleic acid encoding FKRP comprises a modification in each of the 5′ and 3′ UTRs.

In one embodiment of any of the aspects described herein, the modification in the 5′ and/or 3′ UTR of FKRP causes an increase or a decrease in protein expression and/or enzymatic activity upon expression in a cell as compared to the protein expression and/or enzymatic activity of a construct comprising wild type 5′ and 3′ FKRP UTRs under substantially similar conditions.

In one embodiment of any of the aspects described herein, the administration of the AAV vector comprises intravenous and/or intramuscular injection.

In one embodiment of any of the aspects described herein, the subject is a human.

In one embodiment of any of the aspects described herein, at least one symptom of a FKRP-mediated disease or disorder comprises: muscle pain, muscle weakness, muscle fatigue, muscle atrophy, inflammation, decrease in average myofiber diameter in skeletal muscle, loss of ambulation, abnormalities in the brain and/or eyes, eye problems, delay in development, intellectual disability, and seizures.

### BRIEF DESCRIPTION OF THE DRAWINGS

FIGS. 1A-1B is a schematic depicting an exemplary adeno-associated viral vector (AAV) comprising a muscle specific expression cassette and a nucleic acid sequence encoding an FKRP transcript (FIG. 1A). Expression levels of FKRP in muscle cells (e.g., C2C12 cells) and in mouse muscle cells following intramuscular injection (FIG. 1B). FIG. 1B shows initial data relating to the removal of the 5′UTR and the resulting increase in FKRP expression, as well as the 5′UTR truncations made based on structure predictions and splice sites.

FIG. 2 Schematic of the pre-mRNA and mature mRNA of FKRP, illustrating how the 5′UTR is on multiple exons while the entire coding region of the gene exists on a single exon.

FIG. 3 Subset of 30 MSECs from a library of 300 showing relative expression levels from 1/100th to 20× higher than the powerful, ubiquitously expressed CMV promoter.

FIGS. 4A-4C are schematics showing exemplary modifications to human FKRP gene constructs featuring the first 401 bases from FKRP (SEQ ID NO: 33). (FIG. 4A) Shown are four types of DNA/RNA sequence motifs found in the 5′ UTR that affect FKRP RNA and protein expression levels in mammalian cells. The full 5′ UTR contains a G-quadruplex, a pseudo-knot, an IRES and hairpin forming sequences that influence RNA and protein expression. (FIG. 4B) Mutation of the Kozak consensus sequence in the FKRP gene, which alters expression levels by modifying protein translation. (FIG. 4C) Expression of FKRP protein or RNA can be increased by removal of portions of, or all of the 5′ UTR of the FKRP gene. As but one example, a modified Kozak consensus sequence was introduced into the FKRP expression construct and the entire 5′ UTR was removed and in addition, the upstream MSEC was inserted immediately upstream of the Kozak sequence.

FIG. 5 is a schematic depicting exemplary 5′UTR variations encoded into the transgene expressed in AAV6. The truncations are based on secondary structure predictions, determined via RNAfold Web Server and sequence patterns.

FIGS. 6A-6E shows removal of the FKRP 5′- and 3′-UTRs increases FKRP expression. FIG. 6A examines cell lysates from differentiated C2C12 myotubes transduced with either 1×10¹¹ or 1×10¹⁰ vector genomes (vg) of A6.C8mF-FLAG to verify vector expression of mouse FKRP (mFKRP). Labels on the left indicate antibody target (FKRP, Ab607; FLAG, FLAG-HRP, Sigma F7425). FIG. 6B examines FKRP levels in myotubes transduced with untagged AAV6-Ck8e-mFKRP (+UTR) and AAV6-Ck8e-mFKRP (-UTR). FIG. 6C (upper) shows FKRP expression in tibialis anterior (TA) muscles of wild-type mice injected IM with 1×10¹¹ vg AAV6-Ck8e-mFKRP (-UTR) relative to an untreated wild-type mouse and C2C12 myotubes transduced with 1×10¹² vg of same vector; FIG. 6C (lower) shows immunostaining of wild-type tibialis anterior muscle injected with AAV6-CK8e-mFKRP. FKRP, green. Golgi, red (GMI30). FIG. 6D shows a schematic illustration of the AAV-CK8e-humanFKRP (hFKRP) construct used in these studies. FIG. 6E analyzes a transgene construct expressed from vectors pseudotyped with capsids from AAV6 (A6.C8hF), AAV9 (A9.C8hF), or AAVMYO1 (AM.C8hF). Wild-type mice were then treated and tested at various timepoints post-injection to identify potential physiological and histological changes. WT, wild-type.

FIGS. 7A-7I examines whole-body and isolated limb physiology of wild-type mice treated with AAV-hFKRP. FIG. 7A shows the distance run by mice on a treadmill by mice injected with either saline (WT) or 6.4×10¹³ vg/kg of A9.C8hF or AM.C8hF. FIG. 7B shows the distance run normalized to mouse weight. FIG. 7C examines the percent distance changed relative to previous absolute distance measured. FIG. 7D shows forelimb grip strength. FIG. 7E shows forelimb grip strength normalized to mouse weight. FIG. 7F shows the percent forelimb grip strength changed relative to absolute grip strength. FIGS. 7G-7I analyzes repeated cycles of plantarflexion eccentric contraction induced injury (20×) in the right hind limb of mice at 4-, 8-, and 12-weeks post-injection measured by hindlimb torque. Limb was stimulated with 10 mA at a frequency of 100 Hz for 0.4 seconds with a 9 second rest interval between contractions. Significant differences are represented by different letters, shared letter indicate no difference. WT, untreated. (n=3-4)

FIGS. 8A-8E examines cardiac hemodynamics and skeletal muscle mechanics following AAV delivery to wild-type mice. Mice were untreated or injected with 6.4×10¹³ vg/kg A9.C8hF or AM.C8hF. FIGS. 8A-8C shows ultrasound imaging of heart and diaphragm function at 6- and 12-weeks post-injection. FIGS. 8D & 8E examines the force: length relationship of TA and diaphragm muscles from same mice at 18 weeks post-injection. Muscles are stretched end-to-end and optimal length is determined at maximum isomeric twitch force (see Methods). No differences were detected in these data. WT, untreated. (n=3-4)

FIGS. 9A-9B shows AAV-FKRP injection does not increase gastrocnemius muscle susceptibility to contraction-induced injury. Isometric force development in wild-type mice treated with A6.C8hF at doses of 4×10¹³, 2×10¹⁴, and 4×10¹⁴ vg/kg. Shown is the percent of maximum isometric force during 8 rounds of gastrocnemius eccentric contraction in situ at 5-7 weeks (FIG. 9A) and 10-weeks (FIG. 9B) post-injection. No significant differences were observed between any of the groups (shared letter indicate no difference). WT, untreated. (n=3-5)

FIG. 10 shows a schematic illustrating the dystrophin-associated protein complex and the extracellular effects of mutated FKRP.

### DETAILED DESCRIPTION OF THE INVENTION

Provided herein are methods and compositions useful for the delivery of a nucleic acid to induce expression of an RNA transcript for FKRP that comprises a modified 5′ and/or 3′ UTR region. Such modifications to the 5′ and/or 3′ UTRs in combination with muscle-specific (e.g., skeletal and/or cardiac) expression of FKRP show improved delivery of FKRP as a therapeutic, as well as reduced side effects from off-target expression of FKRP. The methods and compositions described herein can be used for the treatment or prevention of an FKRP-mediated disease or disorder.

### Definitions

As used herein, the term “nucleic acid cassette” refers to a nucleic acid sequence comprising a transcriptional regulatory region and a region that encodes an FKRP RNA transcript.

As used herein, the term “transcriptional regulatory region” refers to a nucleotide sequence located upstream of the nucleotide sequence encoding FKRP as described herein and that permits the recruitment of transcriptional machinery and initiation of transcription of an FKRP RNA transcript. At a minimum, a transcriptional regulatory region comprises a promoter (e.g., a tissue-specific promoter, a constitutive promoter etc.). The transcriptional regulatory region can also comprise one or more regulatory elements, such as an enhancer or repressor or binding site elements for transcription factors. In one embodiment, the transcriptional regulatory region comprises a promoter and at least 1 enhancer region (e.g., at least 2, at least 3, at least 4, at least 5 or more). The promoter, enhancer element(s) or repressor element(s) can be nucleic acid sequences that are naturally occurring or can be synthetic. The transcriptional regulatory region can be modified to tune expression of FKRP to a desired level, for example, by using a strong promoter and enhancer to increase FKRP expression or alternatively a weaker promoter (or a strong promoter and a weak repressor element) can be used to tune expression of FKRP to a lower level when desired. Further tuning as desired can be achieved through combination of a given transcriptional regulatory region with modifications of the 5′ and/or 3′ UTR of the encoded transcript, e.g., as described herein.

As used herein, the term “FKRP RNA transcript” refers to a messenger RNA that encodes FKRP and comprises a modified 5′ or 3′ untranslated region (UTR) as compared to the 5′ or 3′ untranslated region of wild-type FKRP. In some embodiments, the modifications to the 5′ and/or 3′ UTR comprise truncation of at least 1 nucleotide (e.g., at least 2, at least 5, at least 10, at least 25, at least 100 nucleotides) or a complete truncation of the 5′ and/or 3′ UTR (i.e., removal of all the nucleotides in a given UTR region). Alternatively, modifications to the 5′ UTR can comprise disruption or deletion of one or more sequences that form secondary structures that influence protein expression (e.g., G-quadruplexes, RNA hairpins, pseudoknots and the like); typically, removal of secondary structures in the 5′ UTR will result in enhanced expression of FKRP by removing structures that can impede binding of translational machinery. Conversely, modifications to the 5′ UTR can comprise inclusion of a new secondary structure (e.g., an RNA hairpin) that partially impedes binding of translational machinery to tune expression of FKRP to a lower level, if desired. Modifications to the 3′ UTR can comprise the addition, removal or modulation of one or more elements, including but not limited to elements affecting transcript stability, addition of or changes to a polyadenylation signal, and truncations of reducing length of the 3′ UTR, etc.

As used herein, the term “disruption of” when used in reference to RNA transcript secondary structures (e.g., G-quadruplexes, RNA hairpins, pseudoknots etc), refers to the removal of (or alternatively the addition of) nucleotides that in turn disrupt the secondary structure. For example, disruption of a G-quadruplex (a G-C rich region) can be achieved by removal of one or more G-Cs in the region or mutation of one or more of the guanines (G) or cytosines (C) to an adenine (A) or uracil (U) to disrupt the G-C base pair-mediated formation of the G-quadruplex. As another example, disruption of an RNA hairpin can comprise nucleotide mutations or small deletions that remove or modify the self-complementary/palindromic sequence that results in RNA hairpin formation. One of skill in the art can identify or predict the mutations or deletions necessary for disrupting RNA hairpin formation based on the current understanding of RNA hairpins in the art and knowledge of the ability of RNA hairpins to tolerate small mismatch regions.

As used herein, the term “operably linked” refers to the placement of components e.g., in an upstream transcriptional regulatory region such that they work in relationship, thereby permitting them to function in their intended manner. For example, a control sequence (such as a promoter or enhancer) is positioned in such a way that expression of a nucleic acid sequence encoding FKRP is under the control of the promoter and/or enhancer sequences.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for a cell or individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The terms “patient”, “subject” and “individual” are used interchangeably herein, and refer to an animal, particularly a human, to whom treatment of an FKRP-mediated disease or disorder, including prophylactic treatment is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.). A subject can have previously received a treatment for an FKRP-mediated disease, or has never received treatment for an FKRP-mediated disease. A subject can have previously been diagnosed with having an FKRP-mediated disease, or has never been diagnosed with an FKRP-mediated disease.

As used herein, a “therapeutically effective amount” or a “therapeutically effective dose” refers to an amount of a nucleic acid cassette or AAV vector as described herein that, when administered to a subject, is sufficient to effect partial or complete treatment of an FKRP-mediated disease or condition in the subject. The amount of a nucleic acid cassette or AAV vector that constitutes a “therapeutically effective amount” will vary depending on the nucleic acid cassette or AAV vector, the condition and severity of the disease, the manner of administration, and/or the age of the subject to be treated, but can be determined routinely by one of ordinary skill in the art having regard to his or her own knowledge and to this disclosure. Accordingly, when a nucleic acid cassette or AAV vector is said to possess “therapeutic efficacy,” this is intended to mean that the nucleic acid cassette or AAV vector is capable of effecting treatment of FKRP-mediated disease or condition in a subject, provided a “therapeutically effective amount” of the nucleic acid cassette or AAV vector is administered under appropriate conditions.

As used herein, “treating” or “treatment” refers to the treatment of an FKRP-mediated disease or condition of interest in a subject (e.g., a human) having the disease or condition of interest, and includes: (i) preventing or inhibiting the disease or condition from occurring in the subject, for example, when the subject is predisposed to the condition, but has not yet been diagnosed as having the condition; (ii) inhibiting the disease or condition, i.e., arresting or slowing its development or progression; (iii) relieving the disease or condition, i.e., causing regression of the disease or condition; and/or (iv) relieving one or more symptoms (e.g., muscle weakness, muscle fatigue, abnormalities in the brain and/or eyes), resulting from the disease or condition or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. Successful treatment can also be assessed by a reduction in the need for medical interventions, reduction in hospital or emergency room visits, reduction in fatigue, or other markers of an improved quality of life.

As used herein, the phrase “reducing at least one symptom of an FKRP-mediated disease or disorder” refers to a reduction in the presence of a given symptom, or severity of a given symptom associated with an FKRP-mediated disease or disorder following treatment with the methods and compositions described herein. In one embodiment, the reduction of at least one symptom can include the prevention or delay of an expected symptom onset in a subject diagnosed with an FKRP-mediated disease and undergoing treatment as described herein. Exemplary symptoms of an FKRP-mediated disease include muscle atrophy (e.g., a decrease in muscle mass), muscle weakness, weak heart rate, muscle fatigue, muscle pain, inflammation, decrease in average myofiber diameter in skeletal muscle, loss of ambulation, abnormalities in the brain and/or eyes, eye problems, delay in development, intellectual disability, seizures, and mortality. In one embodiment, at least one symptom of an FKRP-mediated disease is reduced by at least 10% as assessed using an appropriate standard clinical measures such as MRI, CT scan, X-rays, PET scans, electromyography, muscle biopsy, ECG, and patient self-reporting; in other embodiments, the at least one symptom is reduced by at least 20%, at least 30%, at least 40%, at least 50%, at least 60%, at least 70%, at least 80%, at least 90%, at least 95%, at least 98%, at least 99% or even 100% (i.e., symptom is resolved or is below detectable parameters using a clinical measure). In some embodiments, therapeutic efficacy can be measured by a reduction in hospital visits, a reduction in the duration of hospital stays, reduction in medications or doses of such medications, increased longevity, improved quality of life and the like.

Unless specifically defined otherwise, the technical terms, as used herein, have their normal meaning as understood in the art. The following terms are specifically defined with examples for the sake of clarity.

As used herein, “a” and “an” denote one or more, unless specifically noted.

As used herein “about” refers to a quantity, level, value, number, frequency, percentage, dimension, size, amount, weight, or length that varies by as much as about 30%, about 25%, about 20%, about 15%, about 10%, about 9%, about 8%, about 7%, about 6%, about 5%, about 4%, about 3%, about 2%, about 1% to a reference quantity, level, number, frequency, percentage, dimension, size, amount, weight, or length. In any embodiment discussed in the context of a numerical value used in conjunction with the term “about,” it is specifically contemplated that the term “about” can be omitted.

### FKRP-Mediated Neuromuscular Disorders

Provided herein are methods and compositions that can be used in the treatment of FKRP-mediated diseases or disorders. Fukutin-Related Protein (FKRP) is a glycosyl-transferase localized within the trans-golgi complex. It mediates glycosylation and maturation of a-dystroglycan, an essential component of the dystrophin-glycoprotein complex (DGC). Mutations within FKRP result in decreases in a-dystroglycan glycosylation and lead to the disruption of a critical mechanical link between the extracellular matrix (ECM) and the muscle contractile apparatus.

These mutations to FKRP result in the disease progression of e.g., dystroglycanopathies, which are a collection of diseases resulting from dysfunction of a-dystroglycan. Such diseases include limb girdle muscular dystrophy type 2I/R9, congenital muscular dystrophy (MDC1C), Walker-Warburg syndrome, and muscle-eye-brain disease (MED). Traditionally, treatment utilizes gene replacement therapy, however new strategies are being developed in combination with gene replacement therapy. Such strategies include gene upregulation, gene editing, testing novel vectors and delivery systems for gene delivery, and developing improved “muscle-specific expression cassettes” (MSECs) as described herein, which help to optimize gene expression levels from the vectors for FKRP-centric tissue-specific needs.

### FKRP Modifications and Constructs

Provided herein are methods and compositions that comprise expression of an RNA transcript of FKRP that has a modified 5′ and/or 3′ UTR. Methods and compositions provided herein include muscle-specific expression cassettes (MSECs) that encode such RNA transcripts. In some embodiments, the nucleic acid cassette comprising a transcriptional regulatory region and a nucleic acid encoding an FKRP RNA transcript are inserted into an AAV plasmid or vector.

In certain embodiments, the methods and compositions described herein comprise a modified 5′ UTR and/or 3′UTR region of the FKRP nucleic sequence. Modifications to the 5′ UTR can include truncation of at least 1 nucleotide from the N-terminal end of the 5′ UTR; in other embodiments the truncation is at least 2 nucleotides, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 210, at least 220, at least 230, at least 240, at least 250, at least 260, at least 270, at least 280, at least 290, at least 300, at least 310, at least 320, at least 330, at least 337 nucleotides, (e.g., the entire 5′ UTR) from the FKRP sequence. While one of skill in the art will recognize that the term truncation when applied to the 5′ UTR generally refers to the removal of nucleotide(s) from the 5′ terminal end, it is also specifically contemplated that the 5′ UTR can be modified to remove one or more nucleotides from the 3′ terminal end of the 5′ UTR.

Modifications to the 3′ UTR can include truncation of at least one nucleotide from the 3′ terminal end of the 3′ UTR; in other embodiments, the truncation can comprise removal of at least 2 nucleotides at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990, at least 1000, at least 1010, at least 1020, at least 1030, at least 1040, at least 1050, at least 1060, at least 1070, at least 1080, at least 1090, at least 1100, at least 1110, at least 1120, at least 1130, at least 1140, at least 1150, at least 1160, at least 1170, at least 1180, at least 1190, at least 1200, at least 1210, at least 1220, at least 1230, at least 1240, at least 1250, at least 1260, at least 1270, at least 1280, at least 1290, at least 1300, at least 1310, at least 1320, at least 1330, at least 1340, at least 1350, at least 1360, at least 1370, at least 1380, at least 1390, at least 1400, at least 1410, at least 1420, at least 1430, at least 1440, at least 1450, at least 1460, at least 1470, at least 1480, at least 1490, at least 1500, at least 1510, at least 1520, at least 1530, at least 1540, at least 1550, at least 1560, at least 1570, at least 1580, at least 1590, at least 1600, at least 1610, at least 1620, at least 1630, at least 1637 nucleotides (e.g., all) from the FKRP sequence.

In one embodiment, the modification comprises complete deletion of the 5′ UTR and/or the 3′ UTR.

The wild-type FKRP RNA transcript comprises several secondary structures (e.g., G-quadruplex, hairpins, pseudoknots and the like) in the 5′ UTR that can be deleted or disrupted to enhance binding of transcriptional machinery, thereby increasing FKRP expression. Thus, where increased FKRP expression is desired, disruption or deletion of such secondary structures can be utilized. Conversely, if a lower degree of FKRP expression is desired, such secondary structures can be inserted to partially impede translational machinery, thereby expressing FKRP at lower levels. One of skill in the art can use such secondary structures to modulate and tune the expression of FKRP to a desired level.

In some embodiments, modification of the 5′ UTR of the FKRP RNA transcript comprises the removal of a G-quadruplex, an RNA hairpin, a pseudoknot, or any combination thereof, with the goal of increasing expression of FKRP in a cell.

In other embodiments, modification of the 5′ UTR of the FKRP RNA transcript comprises the insertion of a G-quadruplex, an RNA hairpin, a pseudoknot, or any combination thereof, with the goal of decreasing expression of FKRP in a cell.

In some embodiments, modifications to, or removal of, a given region in the 5′ UTR can be made to improve translation by removal of RNA structures that can impede the association of translational machinery. For example, thermodynamically stable structures such as G-quadruplexes and RNA hairpins in the 5′ UTR of an RNA transcript can result in reduced expression of a gene, such as FKRP. Features found in the 5′ UTR such as pseudoknots, hairpins, and RNA G-quadruplexes, as well as upstream open reading frames (uORFs) and upstream start codons, (uAUGs) mainly inhibit translation. In embodiments where an increase in expression is desired, a modification to the 5′ UTR comprises removal or disruption of a G-quadruplex and/or an RNA hairpin. In one embodiment, the G-quadruplex sequence in the 5′ UTR of human FKRP comprises attgctccaagatggcggcggcggcggcagcg (SEQ ID NO. 9). In another embodiment, the G-quadruplex sequence in the 5′ UTR of murine FKRP comprises tgtacaattgctccaagatggcggcggcggcggcggcggcag (SEQ ID NO. 10).

In one embodiment, disruption of a G-quadruplex can include removal of at least 1 nucleotide, at least 2 nucleotides, at least 5, at least 10, at least 15, at least 20, at least 25, at least 30, or at least 32 (e.g., all) nucleotides of the G-quadruplex sequence (SEQ ID NO. 9 and SEQ ID NO. 10) such that it modulates the expression of FKRP.

In one embodiment, disruption of a G-quadruplex can be performed by substituting adenosine (A) and thymine (T)/uracil (U) residues for guanine (G) and cytosine (C) residues in the G-quadruplex sequence (SEQ ID NO. 9 and SEQ ID NO. 10). Disruptions of the G-quadruplex that in turn result in modifications of FKRP expression are preferred.

In one embodiment, disruption of a G-quadruplex can be made by inserting adenosine (A) and thymine (T)/uracil (U) residues for guanine (G) and cytosine (C) residues in the G-quadruplex sequence (SEQ ID NO. 9 and SEQ ID NO. 10). Disruptions of the G-quadruplex that in turn result in modifications of FKRP expression are preferred.

It is also contemplated that the activity of G-quadruplexes can be modulated using small molecule inhibitors and/or small molecule ligands such that the expression of a cassette is modified.

In one embodiment, disruption of a hairpin can be achieved by introducing or increasing the number of intramolecular base pair mismatches through nucleotide substitution, thereby modulating the expression of FKRP. One of skill in the art will recognize that RNA hairpins can tolerate a small number of mismatches and that appropriate disruption of an RNA hairpin can require including at least 3, at least 4, at least 5, or more mismatches.

In one embodiment, disruption of a hairpin can be achieved by removing or adding at least 1 nucleotide, at least 2, at least 3, at least 4, at least 5, at least 6, at least 7, at least 8, at least 9, at least 10, at least 11, or at least the full nucleotide length of one or both strands of a hairpin, thereby modulating the expression of FKRP.

In one embodiment, disruption of a hairpin can be achieved by inserting non-complementary nucleotides into the hairpin sequence, such that it disrupts the folding or formation of a base-paired stem in the nucleic acid strand, and in turn modulates the expression of FKRP.

In one embodiment, modifications to the 5′ UTR can comprise introducing a hairpin that prevents, at least partially, proteins from being recruited to initiate translation (e.g. a splicing hairpin), thereby producing FKRP at a lower level of expression.

In one embodiment, the methods and compositions provided herein comprise a 5′ UTR with a modification comprising a disrupted or deleted pseudoknot. Exemplary pseudoknots that can be deleted or disrupted include pseudoknots that are classified as either a H-, K-, L-, or M-type of pseudoknot. A pseudoknot can also include long-range pseudoknots. Software that allows the user to predict the formation of pseudoknots in an RNA structure include Pseudo Viewer and CyloFold. (Staple and Butcher, PLOS Biol. 2005 June; 3 (6): e213; Bindewald et al. Nucleic Acids Res. 2010 July; 38; W368-72). In one embodiment, the pseudoknot sequence in the 5′ UTR of human FKRP comprises (SEQ ID NO. 29):

AGCTCAGCTGGGCTGGAACTGCCCTCCTGGAACTCCCCCAGCCTACAACC
TAGGAGGTGCAGGGACTGAGGCTCAGGCCAAATCGCAACTCAGACCCAGT
GAACCCAAGGCCTGAAGAGAATTTGGATTCATT

In one embodiment, disruption of a pseudoknot can be achieved by the removal of at least one stem-loop region (at least 2, at least 3, or more stem-loop regions) of the pseudoknot.

In one embodiment, disruption of a pseudoknot can be achieved by the insertion and/or substitution of nucleotides into the pseudoknot sequence, such that it disrupts the folding of the nucleic acid strand.

In one embodiment, disruption of a pseudoknot can be achieved by increasing mismatches through nucleotide substitution.

In one embodiment, disruption of a pseudoknot can be achieved by a point mutation of one or more nucleotides in the pseudoknot sequence that participates in formation of the pseudoknot structure.

In certain embodiments, modifications can include alteration of a Kozak consensus sequence. The Kozak consensus sequence is a nucleotide motif that functions as the protein translation initiation site in many mRNA transcripts. Varying this region affects the “strength” of the initiation sequence. Alterations (including, but not limited to, substitution, addition or deletion of sequence) of the Kozak consensus sequence in FKRP can result in improved translation and are specifically contemplated for use with the methods and compositions described herein.

### Muscle-Specific Expression Cassettes (MSECs)

MSECs are engineered transcriptional regulatory elements that each have varying specificity for different types of muscle tissue, such as skeletal and cardiac muscle tissue. In some embodiments, an MSEC as described herein can comprise control elements (e.g., MSEC enhancers and promoters) that bind both ubiquitous and/or muscle type-specific transcription factors; and the activity of each MSEC is determined by differences in control element types, sequences, numbers, and linear order within the enhancer and promoter regions. MSECs can be used to avoid toxicity and immune activation that occurs with uncontrolled expression of muscle therapeutic proteins in other sites, as well as to restrict the expression of a desired gene (e.g., FKRP) in muscle (e.g., skeletal and/or cardiac).

In some embodiments, the muscle-specific transcriptional regulatory cassette is derived from an M-creatine kinase enhancer and/or a M-creatine kinase promoter sequence. For example, the muscle-specific transcriptional regulatory cassette can be derived from an M-creatine kinase enhancer with an M-creatine kinase promoter. Furthermore, the muscle-specific transcriptional regulatory cassette can include one or more enhancers derived from conserved regions of muscle creatine kinase and/or a CK8 transcriptional regulatory cassette (SEQ ID NO.: 8).

The muscle-specific transcriptional regulatory cassette can be a muscle-specific CK8 transcriptional regulatory cassette (CK8) or a derivative thereof. CK8 is a non-naturally occurring nucleotide sequence including multiple muscle and non-muscle gene control elements arranged in a miniaturized array. CK8 can provide high or very high transcriptional expression of a predetermined RNA and/or protein in skeletal and cardiac muscle cells. In one embodiment, an MSEC useful for the methods and compositions described herein comprises a modified CK8 transcriptional regulatory cassette (e.g., CK8e).

It is contemplated that excess FKRP may be differentially toxic in cardiac muscle. In some preferred embodiments, MSECs that have at least 3-fold, at least 10-fold, at least 30-fold, at least 100-fold, at least 300-fold, at least 1000-fold less transcriptional activity than CK8e can be used. One who is skilled in the art can determine what MSEC would produce low levels of FKRP. Different MSECs, their makeup, and how their activity is measured are described in, e.g., PCT/US2022/023915, which is incorporated herein by reference in its entirety.

In certain embodiments, the muscle-specific transcriptional regulatory cassette can be a CK8e transcriptional regulatory cassette having at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 100% sequence identity, to the nucleotide sequence of SEQ ID NO.: 8.

SEQ ID NO. 8: (CK8e transcriptional regulatory cassette)

TGCCCATGTAAGGAGGCAAGGCCTGGGGACACCCGAGATGCCTGGTTATA
ATTAACCCAGACATGTGGCTGCCCCCCCCCCCCCAACACCTGCTGCCTCT
AAAAATAACCCTGCATGCCATGTTCCCGGCGAAGGGCCAGCTGTCCCCCG
CCAGCTAGACTCAGCACTTAGTTTAGGAACCAGTGAGCAAGTCAGCCCTT
GGGGCAGCCCATACAAGGCCATGGGGCTGGGCAAGCTGCACGCCTGGGTC
CGGGGTGGGCACGGTGCCCGGGCAACGAGCTGAAAGCTCATCTGCTCTCA
GGGGCCCCTCCCTGGGGACAGCCCCTCCTGGCTAGTCACACCCTGTAGGC
TCCTCTATATAACCCAGGGGCACAGGGGCTGCCCTCATTCTACCACCACC
TCCACAGCACAGACAGACACTCAGGAGCCAGCCAGC

In one embodiment, the MSEC comprises CK8e. Alternative MSECs that exhibit either lower overall transcriptional activities, or lower cardiac muscle activities, can be used and are found at PCT/US22/023915, which is incorporated by reference herein in its entirety.

### AAV Vectors

AAV is a parvovirus which belongs to the genus Dependoparvovirus that is useful for the delivery of therapeutic nucleic acids (e.g., a nucleic acid encoding FKRP). AAV's usefulness stems from its ability to infect a wide range of host cells, including non-dividing cells, as well as its ability to infect cells from different species. AAV has not been associated with any human or animal disease and does not appear to alter the biological properties of the host cell upon integration, thus making it an ideal vector for the delivery of therapeutic nucleic acids.

An “AAV vector,” as that term is used herein, comprises a vector derived from an adeno-associated virus serotype, including without limitation, AAV1, AAV2, AAV3, AAV4, AAV5, AAV6, AAV7, AAV8, and AAV9. In one embodiment, the AAV vector comprises AAV6. As will be appreciated by those of skill in the art, AAV vectors can have one or more of the AAV wild-type viral genes deleted in whole or part, e.g., the rep and/or cap genes, but retain functional flanking ITR sequences. Where functional ITR sequences are necessary for the rescue, replication, and packaging of the AAV virion, in some embodiments, the AAV vectors described herein include at least one functional ITR. The ITRs need not be the wild-type nucleotide sequences, and can be altered, e.g., by the insertion, deletion or substitution of nucleotides, so long as the sequences provide for functional rescue, replication and packaging.

In some embodiments, the AAV vectors described herein are engineered to produce synthetic, modified or recombinant AAV vectors. A “recombinant AAV vector” or “rAAV vector” comprises an infectious, replication-defective virus composed of an AAV capsid protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. Exemplary recombinant AAV (rAAV) vectors include, but are not limited to, an AAVRh74 vector, an AAV8 vector, an AAV9 vector, an AAV6 vector, an AAV7 vector, an AAV218 vector, a NP vector, a NP 66 vector, a NP 22 vector, an AAVpo.1 vector, MyoAAV, and/or an AAVMyo vector. In one embodiment, the AAV vector comprises a MyoAAV vector (Weinmann, J. et al. 2020, Nat Commun 11, 5432). In one embodiment, the AAV vector comprises an AAVMyo vector (Tabebordbar, M. et al. 2021, Cell 184, 4919-4938 e4922; Chamberlain, J. S. 2022, N. Engl. J. Med. 386, 1184-1186).

In various embodiments, the therapeutically effective amount of the pharmaceutical composition can be between about 10¹¹ and about 10¹⁶ vector genomes (vg)/kilogram (kg) subject weight, between about 10¹² and about 10¹⁵ vg/kg subject weight, between about 10¹³ and about 10¹⁴ vg/kg subject weight, between about 10¹² and about 10¹⁴ vg/kg subject weight, between about 10¹² and about 10¹⁵ vg/kg subject weight, between about 10¹² and about 10¹⁶ vg/kg subject weight, between about 10¹¹ and about 10¹⁴ vg/kg subject weight, between about 10¹⁰ and about 10¹⁵ vg/kg subject weight, between about 10¹³ and about 10¹⁵ vg/kg subject weight, between about 10¹⁴ and about 10¹⁵ vg/kg subject weight, between about 10¹³ and about 10¹⁴ vg/kg subject weight, or between about 10¹⁴ and about 10¹⁵ vg/kg subject weight.

In some embodiments, the pharmaceutical composition can be administered intravascularly, intraperitoneally, subcutaneously, or by intramuscular injection.

In some embodiments, it can be beneficial to optimize the level of expression of FKRP in muscle tissue (e.g., skeletal and/or cardiac). In some embodiments, it is important to prevent unintended effects from producing too much FKRP, however still expressing enough FKRP to receive its benefits. Some AAV vectors, when combined with different MSECs, can produce high levels of expression of FKRP. However, other AAV vectors, when combined with different MSECs, can produce much lower levels of expression of FKRP. Titrating the optimal expression of FKRP protein can be achieved by selecting vectors and/or MSECs that produce different levels of protein in particular muscle tissues (e.g., skeletal and/or cardiac). Such titration is well within the skill set of one of skill in the art given the guidance provided herein. As but one example, if a construct comprising a modified 5′-FKRP UTR cDNA is determined to convey high FKRP protein levels, it may well be that the MSEC in combination with the modified UTRs and AAV vector produces too much FKRP and possibly even toxic levels. In order to produce a vector that provides lower levels, an MSEC that produces transcripts in a muscle-specific, but lower level can be used, one or more 5′- or 3′-UTR modifications as discussed herein can be introduced, or a combination of MSEC and 5′- or 3′-UTR modifications can be used to titrate FKRP message and/or protein levels as needed.

### Measuring FKRP Expression Levels

Methods to measure the level of FKRP expression products in a cell or tissue are known to a skilled artisan. Such methods to measure FKRP expression products include, e.g., protein level, include ELISA (enzyme linked immunosorbent assay), western blot, immunoprecipitation, histology; immunohistological staining; and/or immunofluoresence assay using detection reagents such as an antibody or protein binding agents.

In one embodiment, an FKRP antibody is used to measure the expression of FKRP from the nucleic acid cassettes or AAV vectors as described herein. Antibodies for FKRP are commercially available and can be used to measure protein expression levels, (e.g. anti-FKRP (Cat. No. sc-374642; Santa Cruz Biotechnologies, Santa Cruz, CA), anti-FKRP (Cat. No. DCABY-1120; Creative Diagnostics, Shirley, NY). Alternatively, since the amino acid sequences for the targets described herein are known and publicly available at the NCBI website, one of skill in the art can raise their own antibodies against these polypeptides of interest for the purpose of the methods described herein. The amino acid sequences of the polypeptides described herein have been assigned NCBI accession numbers for different species such as human, mouse and rat. In particular, the amino acid sequences of human FKRP and murine FKRP are included herein, e.g. SEQ ID NO: 11 and SEQ ID NO.: 12 respectively.

In some embodiments of any of the aspects, immunohistochemistry (“IHC”) and immunocytochemistry (“ICC”) techniques can be used to detect FKRP expression. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay to detect FKRP expression can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the FKRP-specific polypeptides with respective FKRP-specific proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Other techniques can be used to detect the level of a FKRP-specific polypeptide in a sample. One such technique is the dot blot, an adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is then probed with antibody and labelled secondary antibody as in Western blotting. The intensity of the signal from the detectable label in either format corresponds to the amount of enzyme present, and therefore the amount of polypeptide. Levels can be quantified, for example by densitometry.

In certain embodiments, the FKRP expression products as described herein can be instead determined by determining the level of FKRP-specific messenger RNA (mRNA). Such molecules can be isolated, derived, or amplified from a biological sample, such as a blood sample. Techniques for the detection of mRNA expression is known by persons skilled in the art, and can include, but are not limited to, PCR procedures, reverse-transcription (RT) PCR, quantitative RT-PCR (QRT-PCR), real-time PCR (RT-PCR) methods, quantitative RT-PCR Northern blot analysis, differential gene expression, RNAse protection assay, microarray based analysis, next-generation sequencing; hybridization methods, etc. Such methods are known to those of skill in the art and are not described in detail herein.

In some embodiments of any of the aspects, the level of an FKRP-specific mRNA can be measured by a quantitative sequencing technology, e.g. a quantitative next-generation sequence technology. Exemplary methods of sequencing include, but are not limited to, Sanger sequencing, dideoxy chain termination, high-throughput sequencing, next generation sequencing, 454 sequencing, SOLID sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties. Methods of sequencing a nucleic acid sequence are known to those of skill in the art and are not described in detail herein.

The nucleic acid sequences of the genes described herein have been assigned NCBI accession numbers for different species such as human, mouse and rat. For example, the human FKRP mRNA (e.g. SEQ ID NO: 11) is known. Accordingly, a skilled artisan can design an appropriate primer based on the known sequence for determining the mRNA level of the respective gene.

Nucleic acid and ribonucleic acid (RNA) molecules can be isolated from a particular biological sample (e.g. a mouse) using any of a number of procedures, which are well-known in the art, the particular isolation procedure chosen being appropriate for the particular biological sample. (Roiff, A et al. PCR: Clinical Diagnostics and Research, Springer (1994)).

In some embodiments of any of the aspects, one or more of the reagents (e.g. an FKRP-specific antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. FKRP-specific antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′, 4′, 7′,4,7-hexachlorofluorescein (HEX), 6-carboxy-4′,5′-dichloro-2′,7′-dimethoxyfluorescein (JOE or J), N,N,N′, N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, CA. A reagent can also be detectably labeled using fluorescence emitting metals such as 152Eu, or others of the lanthanide series. These metals can be attached to the reagent using such metal chelating groups as diethylenetriaminepentaacetic acid (DTPA) or ethylenediaminetetraacetic acid (EDTA).

### Pharmaceutical Compositions

An aspect of the disclosure relates to pharmaceutical or biopharmaceutical compositions comprising the nucleic acid cassettes or AAV vectors described herein. The pharmaceutical composition can include a transcriptionally regulatory cassette (e.g., a muscle-specific regulatory cassette) operably linked to a nucleotide sequence encoding fukutin-related protein (FKRP), wherein an RNA transcript when expressed from the nucleotide sequence encoding FKRP comprises a modified 5′ and/or 3′ untranslated region (UTR). In other embodiments, the pharmaceutical composition can comprise an AAV vector comprising a transcriptional regulatory cassette operably linked to a nucleotide sequence encoding fukutin-related protein (FKRP), wherein the FKRP RNA transcript when expressed from the nucleotide sequence encoding FKRP comprises a modified 5′ and/or 3′ untranslated region (UTR).

The composition can be prepared in pharmaceutically acceptable, physiologically acceptable, and/or pharmaceutical-grade solutions for administration to a cell or a subject (e.g., an animal), either alone, or in combination with one or more other modalities of therapy. The formulations may be administered in combination with other agents, such as other proteins, polypeptides, pharmaceutically active agents, etc.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions. Where the methods and compositions are directed to the treatment of muscular diseases or disorders, it will be appreciated that methods that permit delivery, at least in part, to muscle are preferred. For example, intramuscular injection can be used to directly deliver the compositions as described herein to muscle (e.g., skeletal and/or cardiac muscle). While delivery to skeletal muscle is preferred, some delivery to cardiac muscle can also be useful for the methods described herein. Alternatively, one can administer the compositions through intravenous injection, as long as sufficient amounts are localized to muscles to produce a desired effect. In order to increase the amount of a composition (e.g., an AAV vector) to a given site, a targeting moiety can be used that will enhance delivery to muscle (e.g., skeletal and/or cardiac muscle).

Exemplary carriers can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

In one embodiment, the composition is formulated for intramuscular delivery. In one embodiment, the composition is formulated for intracardiac delivery.

Therapeutic compositions contain a physiologically tolerable carrier together with the vectors described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmaceutical composition that contains active ingredients dissolved or dispersed therein is understood in the art and need not be limited based on formulation. Typically, such compositions are prepared as injectable either as liquid solutions or suspensions; however, solid forms suitable for solution, or suspension in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition for use with the methods described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of a vector to be administered herein that will be effective in the treatment of a particular disorder or condition will depend on the nature of the disorder or condition, the expression of the therapeutic agent, and can be determined by standard clinical techniques.

While any suitable carrier known to those of ordinary skill in the art can be employed in the pharmaceutical composition, the type of carrier will vary depending on the mode of administration. Compositions for use as described herein can be formulated for any appropriate manner of administration, including for example, intravenous or intramuscular administration. For parenteral administration, such as intramuscular administration, the carrier preferably comprises water, saline, alcohol, a fat, a wax or a buffer. Alternatively, compositions as described herein can be formulated as a lyophilizate.

The pharmaceutical composition may reduce a pathological effect or symptom of a neuromuscular disorder associated with FKRP in a subject.

### Dosage and Administration

Essentially any method of administration can be used with the methods and compositions described herein that permit intramuscular delivery and expression of FKRP in muscle (e.g., skeletal or cardiac muscle). Typically, the administrative method will comprise delivery of a therapeutically effective amount of a pharmaceutical composition to one or more muscles in a subject to be treated (e.g., intramuscular injection, intracardiac injection, systemic or intravenous injection or infusion). The pharmaceutical composition can include a nucleic acid expression cassette comprising a transcriptional regulatory region operably linked to a nucleotide sequence encoding a fukutin-related protein (FKRP) RNA transcript comprising a modified 5′ and/or 3′ untranslated region (UTR).

The compositions can be administered via any suitable route that permits delivery to muscle tissue (e.g. skeletal and/or cardiac), including but not limited to, locally, subcutaneously, systemically, intravenously, intravascularly, intramuscularly, intracardiac, or via a bolus. The compositions can be encapsulated in liposomes, exosomes, microparticles, microcapsules, nanoparticles, and the like, if so desired. Techniques for formulating and administering therapeutically useful polypeptides are also disclosed in Remington: The Science and Practice of Pharmacy (Alfonso R. Gennaro, et al. eds. Philadelphia College of Pharmacy and Science 2000), which is incorporated herein in its entirety.

Suitable lipophilic solvents or vehicles include fatty oils such as sesame oil, or synthetic fatty acid esters such as ethyl oleate, triglycerides, or liposomes. Aqueous injection suspensions can contain substances that increase the viscosity of the suspension, such as sodium carboxymethyl cellulose, sorbitol, or dextran.

Optionally, the suspension can also contain suitable stabilizers or agents that increase the solubility of the active ingredients, to allow for the preparation of highly concentrated solutions. Alternatively, the active ingredient can be in powder form for constitution with a suitable vehicle, e.g., a sterile, pyrogen-free, water-based solution, before use.

In some embodiments, a therapeutic agent can be delivered in an immediate release form. In other embodiments, the therapeutic agent can be delivered in a controlled-release system or sustained-release system. Controlled- or sustained-release of an active ingredient can be stimulated by various conditions, including but not limited to, changes in pH, changes in temperature, concentration or availability of enzymes, concentration or availability of water, or other physiological conditions or compounds.

In some embodiments, the compositions described herein can be administered via a schedule including continuous administration or intermittent administration. Accordingly, in addition to these general schedules, in some embodiments, the composition can be administered twice a day, once a day, once every other day, once a week, once a month, or another suitable period of administration.

In one embodiment, a pump can be used for administration (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)).

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Treatment using the methods and compositions described herein includes both prophylaxis/prevention of disease onset and therapy of an active disease. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating a disease or disorder in a subject (e.g., a muscle disease or disorder). In one embodiment, the subject can be a mammal. In another embodiment, the mammal can be a human, although the approach is effective with respect to all mammals. The method comprises administering to the subject an effective amount of a pharmaceutical composition comprising vector as described herein in a pharmaceutically acceptable carrier.

The dosage range for the agent depends upon the potency, the expression level of the therapeutic protein and includes amounts large enough to produce the desired effect, e.g., reduction in at least one symptom of the disease to be treated. The dosage should not be so large as to cause unacceptable adverse side effects. Generally, the dosage will vary with the therapeutic composition (e.g., AAV vector vs. plasmid delivery), and with the age, condition, and sex of the patient. The dosage can be determined by one of skill in the art and can also be adjusted by the individual physician in the event of any complication.

In some embodiments, the vectors are administered at a multiplicity of infection (MOI) of at least 5, at least 10, at least 20, at least 30, at least 40, at least 50, at least 100, at least 200, at least 500 or more.

In certain embodiments, the vectors are administered at a titer of at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ viral particles or more.

Repeated administration can be performed as necessary to maintain therapeutic efficacy. As used herein, the term “therapeutically effective amount” refers to an amount of a vector or expressed FKRP that is sufficient to produce a statistically significant, measurable change in at least one symptom of a disease (see “Efficacy Measurement” below). Alternatively, a therapeutically effective amount is an amount of a vector or expressed FKRP protein that is sufficient to produce a statistically significant, measurable change in the expression level of a biomarker associated with the disease in the subject. Such effective amounts can be gauged in clinical trials as well as animal studies for a given agent.

The vector compositions can be administered directly to a particular site (e.g., intramuscular injection). It is also contemplated herein that the agents can also be delivered intravenously (by bolus or continuous infusion), or systemically, if so desired and provided that FKRP can be expressed in one or more muscle sites (e.g., skeletal and/or cardiac).

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration.

Administration of the doses recited above or as employed by a skilled clinician can be repeated for a limited and defined period of time. In some embodiments, the doses are given once a day, or multiple times a day, for example, but not limited to three times a day. Typically, the dosage regimen is informed by the half-life of the agent as well as the minimum therapeutic concentration of the agent in blood, serum or localized in a given biological tissue. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

### Efficacy

In some embodiments, the methods and compositions described herein comprise a step of diagnosing a subject as having an FKRP-mediated disease. FKRP-mediated diseases can be initially diagnosed by a muscle MRI. FKRP-mediated diseases can show damage to the proximal muscles with relative preservation of the muscles of the anterior compartment of the thighs. A follow up with genetic analysis by specific sequencing of the FKRP gene or of a panel grouping together all the genes involved in the glycosylation of a a-dystroglycan, or a larger panel of genes can be used to confirm the diagnosis.

The efficacy of a given treatment for reducing or preventing FKRP-mediated diseases can be determined by the skilled clinician. However, a treatment is considered “effective treatment,” as the term is used herein, if any one or all of the signs or symptoms of muscle (e.g., muscle weakness, muscular atrophy), brain, and/or eye deterioration is/are altered in a beneficial manner, or other clinically accepted symptoms or markers of disease are improved, or ameliorated, e.g., by at least 10% following treatment with a therapeutic agent that increases expression of FKRP in muscle (e.g., skeletal and/or cardiac). Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of muscle, brain, and/or eye deterioration or (2) relieving the disease, e.g., causing regression of symptoms; and (3) preventing or reducing the likelihood of the development of the disease, or preventing secondary diseases/disorders associated with the deterioration of the muscle, brain, and/or eye.

The technology may be as described in any one of the following numbered paragraphs:

Paragraph 1. A nucleic acid expression cassette comprising a transcriptional regulatory region operably linked to a nucleic acid sequence encoding a fukutin-related protein (FKRP) RNA transcript that comprises a modified 5′ and/or 3′ untranslated region (UTR).

Paragraph 2. The nucleic acid expression cassette of paragraph 1, wherein the modified 5′ untranslated region (UTR) is truncated as compared to the 5′ UTR of wild-type FKRP.

Paragraph 3. The nucleic acid expression cassette of paragraph 1, wherein the modified 5′ UTR comprises a deletion of at least one nucleotide, a plurality of nucleotides or all of the nucleotides in the 5′ UTR region.

Paragraph 4. The nucleic acid expression cassette of paragraph 3, wherein the modification of the 5′UTR comprises deletion or disruption of a G-quadruplex, or a hairpin in the 5′ UTR.

Paragraph 5. The nucleic acid expression cassette of paragraph 4, wherein the modification comprises a modification to the Kozak consensus sequence.

Paragraph 6. The nucleic acid expression cassette of paragraph 1, wherein the modified 3′ UTR is truncated compared to the 3′ UTR of wild-type FKRP.

Paragraph 7. The nucleic acid expression cassette of paragraph 5, wherein the modification to the 3′ UTR comprises deletion of at least one nucleotide, a plurality of nucleotides or all of the nucleotides in the 3′ UTR region.

Paragraph 8. The nucleic acid expression cassette of paragraph 1, wherein the nucleic acid encoding FKRP comprises a modification in each of the 5′ and 3′ UTRs.

Paragraph 9. The nucleic acid expression cassette of any of paragraphs 1-8, wherein the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

Paragraph 10. The nucleic acid expression cassette of any one of paragraphs 1-9, wherein the transcriptional regulatory region comprises a muscle-specific expression cassette (MSEC).

Paragraph 11. The nucleic acid expression cassette of paragraph 10, wherein the MSEC is selected from the group consisting of CK8e.

Paragraph 12. The nucleic acid expression cassette of paragraph 11, wherein upon administration to a cell, expression level of an FKRP mRNA or protein is higher when operably linked to an MSEC than the expression level of the FKRP mRNA or protein when operably linked to a CK8e transcriptional regulatory region.

Paragraph 13. The nucleic acid expression cassette of paragraph 11, wherein upon administration to a cell, expression level of an FKRP mRNA or protein is lower when operably linked to an MSEC than the expression level of the FKRP mRNA or protein when operably linked to a CK8e transcriptional regulatory region.

Paragraph 14. An RNA transcript generated by transcription of the nucleic acid expression cassette of any one of paragraphs 1-13.

Paragraph 15. An adeno-associated viral vector (AAV) comprising the nucleic acid expression cassette of any one of paragraphs 1-13.

Paragraph 16. The AAV vector of paragraph 15, wherein the adeno-associated viral vector is selected from the group consisting of: an AAVRh74 vector, an AAV8 vector, an AAV9 vector, an AAV6 vector, an AAV7 vector, an AAV218 vector, a NP vector, a NP 66 vector, a NP 22 vector, an AAVpo. 1 vector, a MyoAAV vector, and an AAVMyo vector.

Paragraph 17. The AAV vector of paragraph 15, wherein the adeno-associated viral vector comprises an internal terminal repeat (ITR), a muscle-specific expression cassette, a nucleic acid encoding FKRP, a polyadenylation signal (pA+), and/or a second ITR.

Paragraph 18. An engineered cell comprising or expressing a nucleic acid expression cassette of any one of paragraphs 1-17.

Paragraph 19. The engineered cell of paragraph 18, wherein the modified 5′ untranslated region (UTR) is truncated as compared to the wild-type 5′ UTR of FKRP.

Paragraph 20. The engineered cell of paragraph 18 or 19, wherein the modified 5′ UTR comprises a deletion of at least one nucleotide, a plurality of nucleotides or all of the nucleotides in the 5′ UTR region.

Paragraph 21. The engineered cell of paragraph 20, wherein the modification of the 5′UTR comprises deletion or disruption of a G-quadruplex, or a hairpin in the 5′ UTR.

Paragraph 22. The engineered cell of paragraph 20 or 21, wherein the modification comprises a modification to the Kozak consensus sequence.

Paragraph 23. The engineered cell of paragraph 18, wherein the modified 3′ UTR is truncated compared to the 3′UTR of wild-type FKRP.

Paragraph 24. The engineered cell of paragraph 23, wherein the modification to the 3′ UTR comprises deletion of at least one nucleotide, a plurality of nucleotides or all of the nucleotides in the 3′ UTR region.

Paragraph 25. The engineered cell of paragraph 18, wherein the nucleic acid encoding FKRP comprises a modification in each of the 5′ and 3′ UTRs.

Paragraph 26. The engineered cell of any of paragraphs 18-25, wherein the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

Paragraph 27. A method of expressing an FKRP gene product in a subject comprising administering an adeno-associated viral vector of any one of paragraphs 15-17 to a subject in need thereof.

Paragraph 28. The method of paragraph 27, wherein the FKRP gene product is a RNA transcript and/or a protein.

Paragraph 29. The method of claim 27, wherein the subject in need thereof comprises limb girdle muscular dystrophy type 2I/R9 (LGMD2i), Walker-Warburg syndrome, or muscle-eye-brain disease (MED).

Paragraph 30. The method of paragraph 27, wherein the modified 5′ untranslated region (UTR) is truncated as compared to the 5′ UTR of wild-type FKRP.

Paragraph 31. The method of paragraph 27, wherein the modified 5′ UTR comprises a deletion of at least one nucleotide, a plurality of nucleotides or all of the nucleotides in the 5′ UTR region.

Paragraph 32. The method of paragraph 31, wherein the modification of the 5′UTR comprises deletion or disruption of a G-quadruplex, or a hairpin in the 5′ UTR.

Paragraph 33. The method of paragraph 32, wherein the modification comprises a modification to the Kozak consensus sequence.

Paragraph 34. The method of paragraph 27, wherein the modified 3′ UTR is truncated compared to the 3′ UTR of a wild-type FKRP.

Paragraph 35. The method of paragraph 34, wherein the modification to the 3′ UTR comprises deletion of at least one nucleotide, a plurality of nucleotides or all of the nucleotides in the 3′ UTR region.

Paragraph 36. The method of claim 27, wherein the nucleic acid encoding FKRP comprises a modification in each of the 5′ and 3′ UTRs.

Paragraph 37. The method of any of paragraphs 27-36, wherein the modification in the 5′ and/or 3′ UTR of FKRP causes a reduction or inhibition in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

Paragraph 38. The method of paragraph 27, wherein the administration of the AAV vector comprises intravenous and/or intramuscular injection.

Paragraph 39. The method of paragraph 27, wherein the subject is a human.

Paragraph 40. A method for reducing at least one symptom of an FKRP-mediated disease or disorder, the method comprising administering an AAV vector of any one of paragraphs 15-17 to a subject in need thereof, thereby reducing at least one symptom of an FKRP-mediated disorder.

Paragraph 41. The method of paragraph 40, wherein the FKRP-mediated disease or disorder comprises limb girdle muscular dystrophy type 2I/R9 (LGMD2i), Walker-Warburg syndrome, or muscle-eye-brain disease (MED).

Paragraph 42. The method of paragraph 40, wherein the modified 5′ untranslated region (UTR) is truncated as compared to the wild-type 5′ UTR of FKRP.

Paragraph 43. The method of paragraph 40, wherein the modified 5′ UTR comprises a deletion of at least one nucleotide, a plurality of nucleotides or all of the nucleotides in the 5′ UTR region.

Paragraph 44. The method of paragraph 43, wherein the modification of the 5′UTR comprises deletion or disruption of a G-quadruplex, or a hairpin in the 5′ UTR.

Paragraph 45. The method of paragraph 44, wherein the modification comprises a modification to the Kozak consensus sequence.

Paragraph 46. The method of paragraph 40, wherein the modified 3′ UTR is truncated compared to the 3′ UTR of a wild-type FKRP.

Paragraph 47. The method of paragraph 46, wherein the modification to the 3′ UTR comprises deletion of at least one nucleotide, a plurality of nucleotides or all of the nucleotides in the 3′ UTR region.

Paragraph 48. The method of paragraph 40, wherein the nucleic acid encoding FKRP comprises a modification in each of the 5′ and 3′ UTRs.

Paragraph 49. The method of any of paragraphs 40-48, wherein the modification in the 5′ and/or 3′ UTR of FKRP causes an increase or a decrease in protein expression and/or enzymatic activity upon expression in a cell as compared to the protein expression and/or enzymatic activity of a construct comprising wild type 5′ and 3′ FKRP UTRs under substantially similar conditions.

Paragraph 50. The method of paragraph 40, wherein the administration of the AAV vector comprises intravenous and/or intramuscular injection.

Paragraph 51. The method of paragraph 40, wherein the subject is a human.

Paragraph 52. The method of paragraph 40, wherein at least one symptom of a FKRP-mediated disease or disorder comprises: muscle pain, muscle weakness, muscle fatigue, muscle atrophy, inflammation, decrease in average myofiber diameter in skeletal muscle, loss of ambulation, abnormalities in the brain and/or eyes, eye problems, delay in development, intellectual disability, and seizures.

### EXAMPLES

It will be readily understood that the embodiments, as generally described herein, are exemplary. The following more detailed description of various embodiments is not intended to limit the scope of the present disclosure, but is merely representative of various embodiments, Moreover, the order of the steps or actions of the methods disclosed herein may be changed by those skilled in the art without departing from the scope of the present disclosure. In other words, unless a specific order of steps or actions in required for proper operation of the embodiment, the order or use of specific steps or actions may be modified.

### Example 1

Mutations in the gene encoding fukutin-related protein (FKRP) lead to limb girdle muscular dystrophy type 2I/R9, and less frequently to congenital muscular dystrophy (MDC1C), Walker-Warburg syndrome (WWS) and rarely muscle-eye-brain disease (MEB). Provided herein are methods and compositions for delivering FKRP to a muscle cell. In some embodiments, FKRP is delivered to a cell by way of expression from a nucleic acid expression cassette. Such nucleic acid expression cassettes can be encoded in an AAV vector.

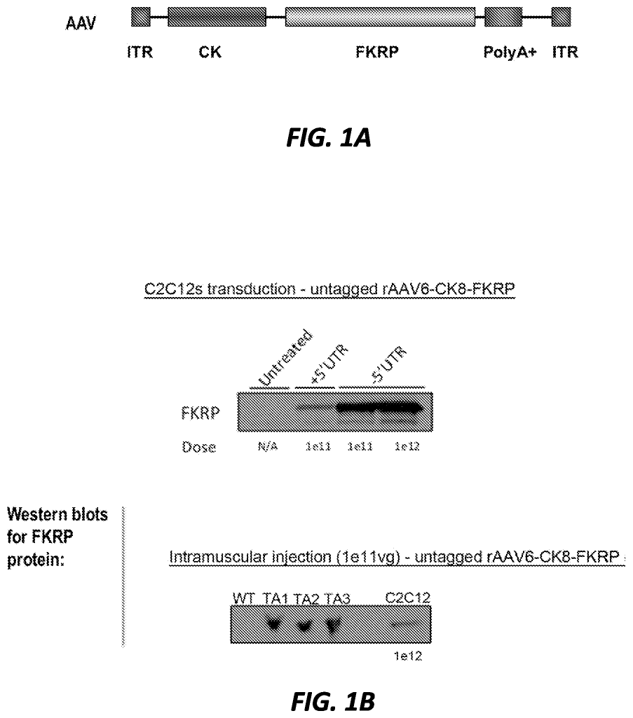

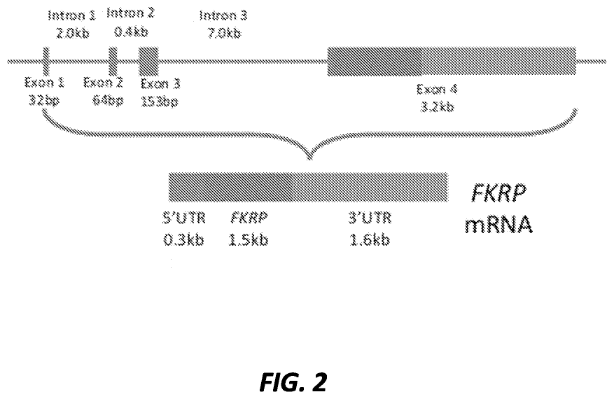

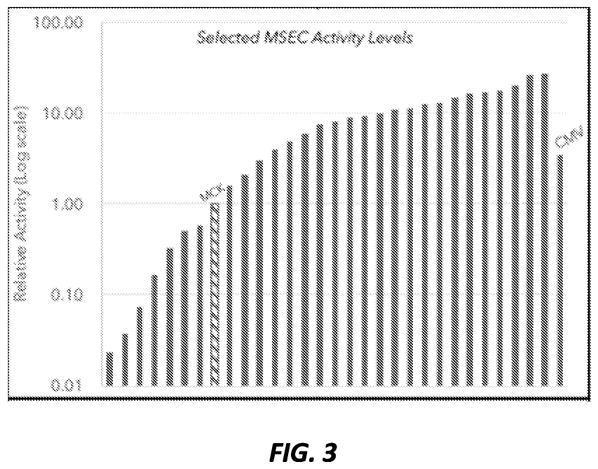

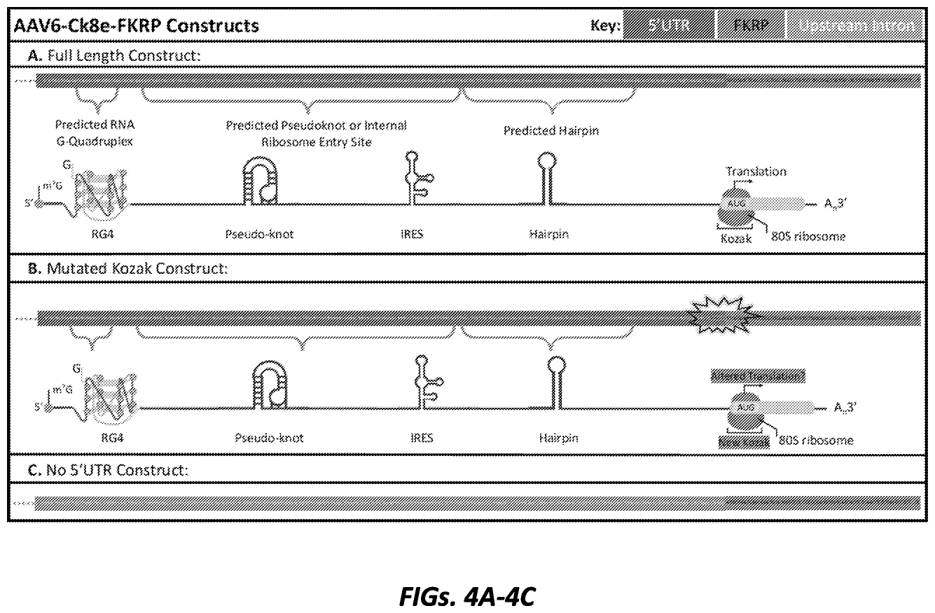

The inventors have worked to optimize a vector system with the goal of achieving maximal functional benefit with the lowest possible dose. Exemplary vector backbones are shown in FIG. 1A and FIG. 2. Typically, an AAV plasmid used in the methods and compositions described herein comprises ITRs, a muscle-specific expression cassette (e.g., CK8e), the wild-type FKRP cDNA and a polyA signal. This vector can be adapted as desired and tested using initial dose escalation studies, for example, with the AAV6 capsid.

The AAV vectors can be assessed and compared at varying intravascular doses between 5×10\hat{ }12 vector genomes per kg (vg/kg) up to 2×10\hat{ }14 vg/kg in different models. Without wishing to be bound by theory, the low dose of ˜5×10\hat{ }12 vg/kg may not be particularly useful for systemic delivery but can still be considered for direct administration to muscle. The high dose of ˜2×10\hat{ }14 vg/kg is the dose that can be used to obtain near saturating levels of gene transfer in several different models of muscular dystrophy, and is the dose being used in essentially all current human gene therapy trials for DMD, LGMD, SMA and MTM1. Thus, in one embodiment, a dose of 2×10\hat{ }14 vg/kg of a given AAV vector is used with the methods and compositions described herein.

It is specifically contemplated herein that additional studies can be designed to test and optimize the expression cassette needed for clinical gene transfer. Importantly, ongoing human gene therapy trials have highlighted the critical need for refined MSECs to appropriately target gene expression, while avoiding toxicity and immune activation.

### Example 2: FKRP Sequence Optimization

Analysis of the untranslated (UTR) regions of the FKRP gene indicates the presence of inhibitory sequences in the 5′ UTR of the FKRP gene. These include high GC content, an RNA G-quadruplex structure and other inverted repeats. Removal of these sequences in expression vectors as described herein has led to increased FKRP expression levels.

The inventors have generated and tested expression cassettes with increasing truncations of the 5′ UTR in combination with altered Kozak consensus sequences to determine which regions maximally impact FKRP expression. In the context of FKRP gene delivery, these results can help identify ways to either enhance or dampen expression levels depending on whether the studies identify safety/toxicity/functionality issues with under- or over-expression of the native gene.

Finally, the inventors have explored truncation of the large 3′ UTR of the FKRP gene with a goal of identifying potential regulatory sequences, which, if removed, permits shortening of the expression cassette to include auxiliary sequences for delivery or for tighter control of expression levels via MSECs.

It is specifically contemplated herein that the sequences described herein can be codon-optimized for human use. Final designs can be tested in animal models, preferably the rat or mouse models.

### Example 3: Selection of AAV Serotypes

Optimal gene therapy for LGMD2I and CMD can benefit from vectors that display enhanced targeting of striated muscles and muscle satellite (stem) cells, while minimizing transduction of the liver and other organs. Ongoing work in the field aims to develop synthetic capsids to either alter tropism or evade pre-existing immunity to natural serotypes.

Three types of synthetic capsids have been developed:
(a) insertion of short sequences from one serotype to another,
(b) synthesis of novel capsids following ancestral AAV sequence reconstruction
(c) shuffling sequences randomly and between different natural serotypes.

Specific subsequences in these serotypes can also be shuffled between different capsids to further refine targeting, as can any promising ligands emerging from the nucleocapsid screens.

### Example 4: Immunological Considerations

One precaution with all exogenous therapeutics for genetic disorders is activation of immune responses that can limit therapies or lead to toxic adverse events such as liver dysfunction and thrombocytopenia. In some embodiments, the number or extent of immune events is monitored and approaches can be developed to ameliorate their occurrence.

Efforts to limit immunity include the use of muscle-restricted (MSEC-mediated) gene expression, which has been shown to largely eliminate immune responses against foreign proteins. For example, by limiting expression to muscle, exposure of the modified genes and their products to antigen-presenting cells is limited.

Monitoring immune responses can be performed using established assays for neutralizing antibody titers, cytokine release, complement activation, blood cell counts and chemistries and activation of cellular immune responses.

### Example 6: Testing Gene Delivery Systems

A wide variety of testing systems can be used to evaluate the various vector and other gene delivery systems contemplated herein. These include conventional mouse models of FKRP disorders using dose escalation combined with functional and immunological testing. However, the inventors can also test expression cassettes and vectors in various in vitro systems, such as myogenic cultures (primary and iPSC-derived), 2D and 3D human myogenic systems as discussed elsewhere herein.

The in vitro systems permit analysis of efficiency, expression levels and some functional readouts. The in vivo systems can also add tropism, efficiency, whole body function and safety studies including immune responses, serum chemistries, blood counts and complement activation.

In some embodiments, the gene therapy vectors can be optimized to express FKRP at varying levels in muscle cells. The therapeutically effective amount of FKRP expression can be affected by the percentage of muscle fiber and cardiomyocyte myonuclei that are transduced, which varies by the use of different therapeutic vectors; and since new vector designs will improve transduction efficiencies, optimal therapeutic protein product levels may decrease as targeted transduction efficiencies improve.

MSECs with attributes for treating LGMD2I have been designed and are currently being optimized, and can be tested for efficacy in FKRP mutant mouse and rat models. This work can be done in the following three exemplary phases.

Phase 1 testing can entail a series of studies starting with the AAV gene therapy approaches outlined above. AAV-FKRP vectors can be tested using MSECs that are expected to produce low, medium and high levels of expression. A first goal can be to identify the strongest MSECs that generate therapeutic efficacy without toxicity. If toxicity is observed, the focus can be shifted to weaker MSECs.

The Phase 2 testing can compare a series of MSEC cassettes in the general range of activity below those associated with adverse effects and focus on obtaining uniform expression levels in as many skeletal muscles and fiber types as possible, while also allowing good expression levels in cardiac muscle. These studies can involve sequential testing initially with reporter genes, followed by confirmatory and functional studies with the FKRP gene.

Phase 3 testing can be performed to optimize MSECs for the potential expression of FKRP having modified 5′ and/or 3′ UTRs as described herein for up-regulating the expression of mutant FKRP in genetic situations in which LGMD2I patients produce low-activity FKRP. Each of these strategies can benefit from the use of existing MSECs, or pairing with additional MSECs, with transcriptional activities that are appropriate for the particular therapeutic strategy.

### Example 7: Cardiac and Skeletal Muscle Performance

For heart function, the inventors assess longitudinal systolic and diastolic performance by M-mode and Tissue Doppler echocardiographic imaging. Endpoint hemodynamics is assessed in situ by Millar catheter and in vitro by Langendorff perfusion. For skeletal muscle, in situ performance is assessed by direct nerve or zonal stimulation of rodent hindlimb muscle. For excised skeletal muscle, the inventors assess twitch and tetanic contraction, fatigue resistance and recovery. Intracellular calcium can be measured by microscopy after loading muscle with fluorescent calcium chelators.

### Example 8: Demembranated Muscle and Isolated Myofibril Contractile Properties

Dissolving the surface membrane of skeletal or cardiac muscle cells permits study of the contractile apparatus performance without the confounding influence of intracellular calcium dynamics that occurs during contractions. Tissue and single muscle cell preparations are robust, allowing the inventors to test several conditions in each preparation. Isolated myofibrils (single sub-cellular contractile organelles) allow study of the millisecond timescale kinetics of contractile activation and relaxation, with high fidelity and resolution of forces in the piconewton range.

### Example 9: Muscle Tissue, Cell and Myofibril Structure

Tissue-level structural analysis permits the examination of fibrosis, satellite cells and morphological features such as centralized nuclei and muscle damage. In muscle cells, the contractile units called sarcomeres align in the direction perpendicular to the long axis of the cell within myofibrils, and the boundaries of these structures (z-disks) align between myofibrils for high-order parallelization of the contractile apparatus. This order is often disrupted in diseased and damaged muscle, contributing to reduction of force production, altering contraction and relaxation kinetics, and furthering downward spiral muscle damage. Within individual myofibrils, electron microscopy can be used to examine the detailed structure of sarcomere thin and thick filaments and z-disks, and these approaches can be used to study structural effects of therapeutic interventions.

### Example 10: Isolated Contractile Protein Mechanics

An advantage in flow cell assays of isolated contractile protein mechanics is that effects on myosin, actin, troponin and tropomyosin can be assessed as the molecular target for therapeutics, and a large number of conditions can be rapidly assessed since proteins can be separately treated once they are all in a flow cell assay.

### Example 11: Metabolic Profiling

Targeted aqueous metabolite profiling analysis can be performed using, for example, the Agilent 1260/AB-Sciex 5500 Qtrap LC-MS/MS instrument and HILIC (hydrophilic interaction chromatography) protocols. The development of highly multiplexed, targeted methods using advanced LC-MS/MS instruments provides fast data acquisition and reasonably tight quality control, resulting in reproducible measurement of >200 metabolites located in >60 different metabolic pathways.

### Example 12: Mitochondrial Function

Isolated mitochondria and permeabilized cardiac tissue can undergo high-resolution respirometry (HRR) via Oxygraph for the measurement of respiration/respiratory complex analysis (oxygen consumption and flux), mitochondrial membrane potential, ROS and ATP production. Mitochondrial content can be assessed by Western blot to assay ETS (Mitosciences Oxphos profile) and VDAC (Santa Cruz Biotech) protein expression, citrate synthase activity and mtDNA/nuclear DNA. ATP synthesis rate in beating hearts can be assessed, if needed, using 31P NMR transfer compared to estimation by MVO2.

### Example 13: Efficacy and Muscle Safety Assessment of Fukutin-Related Protein Gene Therapy

### Introduction

Dystroglycanopathies are a family of muscle disorders (>20) that are caused by altered glycosylation of α-dystroglycan (α-DG), a peripheral membrane protein located on the extracellular side of the sarcolemma that normally binds to laminin. The laminin-α-DG association is a crucial portion of the dystrophin-glycoprotein complex (DGC), which provides a mechanical link between the intracellular actin cytoskeleton and the extracellular matrix. The DGC enables the lateral transmission of forces from within myofibers, allowing the muscle bundle to contract in unison and preventing cellular damage by internally maintained contractile energy. More than 11 glycosyltransferases are known to post-translationally modify α-DG, working sequentially to build long glycan chains onto the protein. One such glycosyltransferase, Fukutin-related protein (FKRP), catalyzes the transfer of ribitol 5-phosphate to a phosphorylated O-mannosyl trisaccharide on α-DG, but only after Fukutin has added a ribitol 5-phosphate to the growing chain. These sequential modifications lead up to the addition of a repeating glucuronic acid and xylose chain that serves as the laminin binding domain of α-DG.

Altered α-DG glycosylation severely disrupts the DGC mechanics, leading to fragile sarcolemma membranes and muscular dystrophy. These resulting dystroglycanopathies are the primary cause for several forms of congenital muscular dystrophy as well as multiple limb-girdle muscular dystrophies. Limb-girdle muscular dystrophy type R9 (LGMDR9, previously LGMD21) is one of the most common of these diseases. It is an autosomal recessive disorder caused by mutations in the FKRP gene which also can lead to congenital muscular dystrophy (MDC1C), Walker-Warburg syndrome (WWS) and muscle-eye-brain disease (MEB). The study is focused on developing treatment options for LGMDR9, although the results will also be relevant to the other, less prevalent FKRP disorders. LGMDR9 is slowly progressive, but patients still experience symptoms such as muscle weakness, muscle cramps, hypertrophy, joint contractures, and, in some cases, severe cardiomyopathy and respiratory issues. The age of LGMDR9 onset varies, with a spectrum of symptoms presenting in relation to specific mutations in FKRP. For example, affected LGMDR9 patients are often wheelchair-dependent by 25 years after age of onset. Diagnoses are typically made based on elevated serum creatine kinase (CK) levels and proximal muscle weakness followed by genotyping. There is no cure for LGMDR9, and treatments are limited to temporary symptom amelioration.

LGMDR9 is often due to heterozygous and homozygous mutations in the 1.5 kb coding region of the FKRP gene, the most common of which is 826C>A (L276I). There is a strong genotype-phenotype correlation for this mutation with compound heterozygous patients displaying a more severe phenotype than homozygous patients. Another common mutation is 1343C>T (P448L), and both mutations interfere with the transfer of FKRP from the endoplasmic reticulum to the Golgi apparatus. As FKRP is a post-translational glycosyltransferase, mislocalization of this enzyme leads to decreased glycosylation and half-life, and results in increased targeting of α-DG by the proteosome. Additionally, other insertion, deletion, missense, and nonsense mutations have been reported in patients, though less commonly than the L276I and P448L point mutations. Interestingly, FKRP-null mutations are embryonic lethal, which explains why all patients genotyped to date have at least one mutant allele that leads to expression of a presumably partially functional protein. These and other data suggest that most, if not all, mutant FKRP enzymes found in patients still retain some enzymatic activity.

Developing approaches for gene therapy of LGMDR9 have been promising. However, inconsistent vectors, mouse age, genotype, and transgene have resulted in contradictory toxicity evidence associated with FKRP-overexpression. For example, recent data have shown that adeno-associated viral vector (AAV)-mediated systemic delivery of FKRP can significantly ameliorate the dystrophic phenotype in a murine disease model, the FKRP^{P448L} mouse, which will reasonably recapitulate LGMDR9. Mouse age varied widely across the studies and the regulatory cassettes used in the various studies similarly differed, as some groups used the strong and ubiquitously expressed CMV (human cytomegalovirus immediate early enhancer plus promoter) or CB (CMV enhancer/chicken β-actin promoter) cassettes while others used a muscle-specific creatine kinase (CK7) cassette. The use of the mouse vs. the human FKRP cDNA also varied between studies, as did doses that ranged from 2×10¹² vg/kg to 6×10¹³ vg/kg. The ability of gene therapy to treat LGMDR9 as well as its durability are also significantly dose-dependent, with lower doses displaying shorter-term, but still significant effects.

Moreover, only one test of systemic muscle function has been performed on FKRP^{P448L} mice receiving FKRP gene therapy, a treadmill exhaustion assay that, although useful, provides limited metrics for analysis. Other tests of systemic muscle function include running wheels for voluntary exercise and treadmills with respiratory chambers for assessing metabolic rate during forced exercise, all of which are recommended assessments for preclinical studies with dystrophic mice. Quantifying metabolic rate through indirect calorimetry assesses the combined functional integrity of both skeletal and cardiac muscle and differs from plethysmography. This latter measure of ventilation reflects diaphragm function only and has little effect on metabolic rate. Exercise-based assessments mimic those often used to assess dystrophic patients in the clinic (e.g., 6-minute-walk test) and can be used to exacerbate the dystrophic phenotype in preclinical studies.

Therapeutic delivery method is yet another component that should be considered in the development of the safest possible therapeutic for LGMDR9 and related CMDs. Increased gene expression is often a critical part of gene therapy, as therapeutics are limited by the immune responses associated with high doses of AAVs in human patients. For example, the issue of systemically administered AAVs and the accompanying liver toxicity seen in clinical trials remain a barrier moving forward. Therefore, an ideal treatment would maximize gene expression while minimizing the AAV dose. The latest advancements in AAV vector designs have led to increased targeting and gene expression in specific tissues. The development of these capsid variants that permit muscle-specific gene delivery may alter dosing thresholds and therapeutic efficacy, and need to be examined in relation to current vectors used in the clinic. One such novel AAV capsid has been named AAVMYO1 for its myotropic properties, and has been shown to increase efficiency and specificity in heart, diaphragm, and skeletal muscle. These new developments not only address the issue of myotropism but also decrease liver-tropism, which will be critical to avoid the issues of innate immunity, and will affect the success of gene therapies using high dose AAV vectors.

While it has been shown that FKRP gene therapy is generally an effective treatment in mouse models for LGMDR9, there has been little focus on transgene designs. Transgene optimization provides a tool to improve efficacy and lower necessary treatment doses. Evidence of potential secondary structures was discovered in the untranslated regions of FKRP mRNA. One particularly relevant structure is an RNA G-quadruplex (RGQ), a stable secondary mRNA configuration associated with the inhibition of translation. Repeats of a (CGG) motif in the 5′UTR of FKRP are suggestive of an RGQ, and further investigation into UTR regions can provide a tool for tunable gene expression (for FKRP and other genes).

The current study tested the dose-dependent efficacy of a novel FKRP gene therapeutic. This particular therapeutic differed from the vectors previously tested as it used AAV6 instead of AAV9, as well as a miniaturized mouse muscle creatine kinase enhancer/promoter (CK8e) that is uniquely active and specific for striated muscle (AAV6-Ck8e-humanFKRP, A6.C8hF). To address the issue of potential FKRP toxicity, it was explored whether overexpression of FKRP in wild-type mice affected muscle physiology. These studies delivered the small FKRP cDNA using multiple doses of vectors pseudotyped with capsids from AAV6, AAV9, and the newer myotropic AAVMYO1. Cardiac and muscle physiology assays did not identify any adverse functional affects due to the exogenous FKRP in wild type mice when delivered with any of these serotypes.

### Results

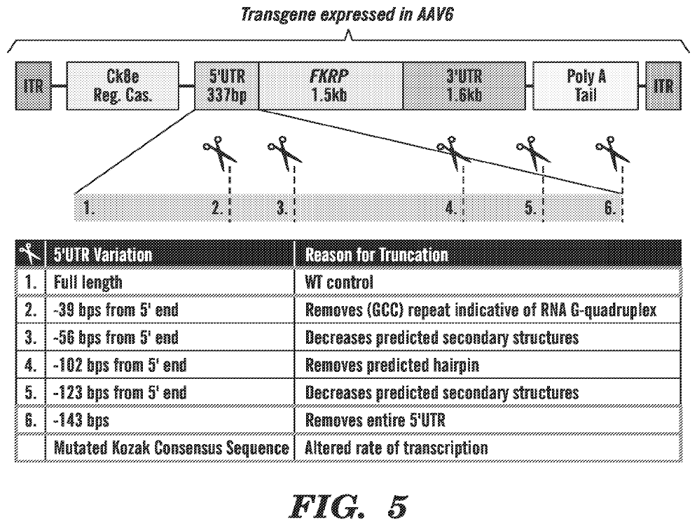

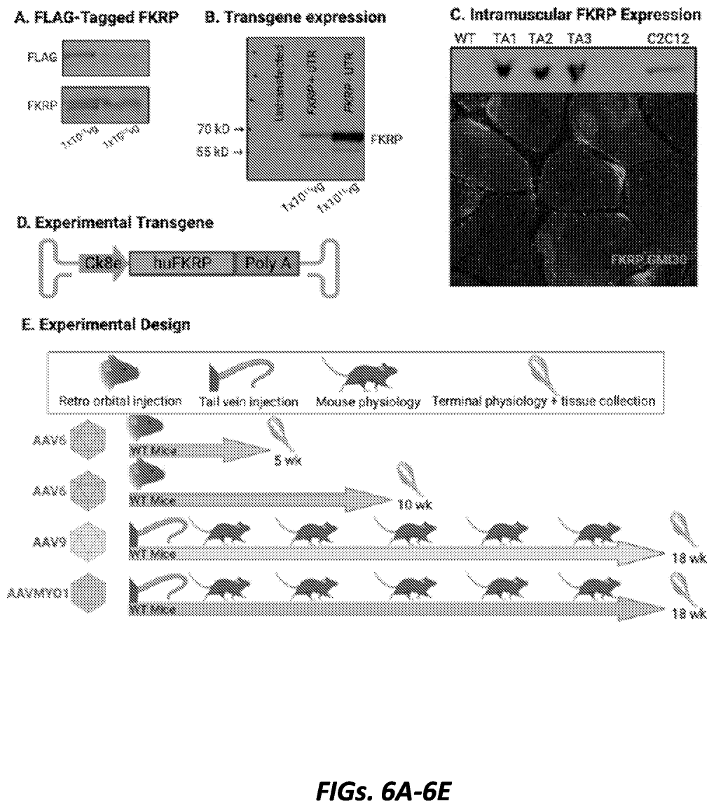

Removal of the untranslated regions from the FKRP cDNA increases protein expression. To explore FKRP expression, rabbit polyclonal antisera (named Ab607) was initially generated against a conserved fragment of the C-terminus of mouse and human FKRP (see methods). To verify the utility of the antisera after affinity-purification, C2C12 myotubes were transduced with 1×10¹² vg, 1×10¹¹ vg, or 1×10¹⁰ vg of AAV6-CK8e-mFKRP-FLAG (the murine Fkrp cDNA with a C-terminal FLAG-tag). Cell lysates revealed co-immunoreactivity with the FKRP and FLAG antibodies and provided confirmation of FKRP production by the AAV6-CK8e-FKRP vector (FIG. 6A). To maximize protein expression, potential inhibitory sequences were located in the FKRP untranslated regions. Sequence analysis suggested there may be regulatory secondary mRNA structures, so both UTRs were removed from the Fkrp cDNA (A6.C8mF). Note, however, that CK8e, which is present in all these vectors, carries 49 bp from the mouse CKM 5′ UTR and that the polyA sequence used was also identical in all vectors (see methods). The effects of these modifications were then compared in vitro by transducing C2C12 myotubes with vectors either carrying or lacking both the Fkrp 5′ and 3′ UTRs. Western analysis with Ab607 showed a notable increase in FKRP expression upon the removal of the UTRs (FIG. 6B). It is important to note that the minor bands at 55 kDa correspond to the predicted molecular weight of FKRP, while the larger bands at ˜60 kDa are likely the N-glycosylated form of the protein. This vector was then tested in vivo via intramuscular injections into the tibialis anterior (TA) of wild-type mice (1×10¹¹ vg/muscle). Analysis of injected muscles revealed FKRP expression by western blot as well as mosaic distribution of the protein throughout the injected muscles (FIG. 6C).

To explore potential deleterious effects from high levels of exogenous FKRP expression, a similar vector was generated that expressed the human FKRP (also lacking the FKRP 5′ and 3′ UTRs). This CK8e-hFKRP construct was encapsulated into AAV6 (A6.C8hF), AAV9 (A9.C8hF), or AAVMYO1 (AM.C8hF) vectors and tested via systemic delivery (FIGS. 6D and 6E).

Wild-type animals treated with AAV-FKRP vectors displayed normal muscle physiology. To explore multiple approaches for delivering FKRP to skeletal muscles and simultaneously monitor for toxic effects, additional assays were performed in wild-type mice injected with vectors made with AAV6, AAV9 or AAVMYO1 capsids. These studies also provided a way to examine the effects of exogenous FKRP in muscle already expressing normal levels of the enzyme. In particular, one previous report suggested that overexpression of FKRP could impair the formation of a functional DGC. The studies used AAV6, as above, but also 2 additional vector types. Gene therapeutics featuring AAV9 capsids are commonly used in clinical trials for different neuromuscular disorders while the recently developed AAVMYO1 was reported to provide significantly enhanced muscle transduction. By using AAV vectors pseudotyped with multiple capsids, including a potent myotropic capsid (AAVMYO1), the results were not limited to the effects of a single type of AAV capsid.

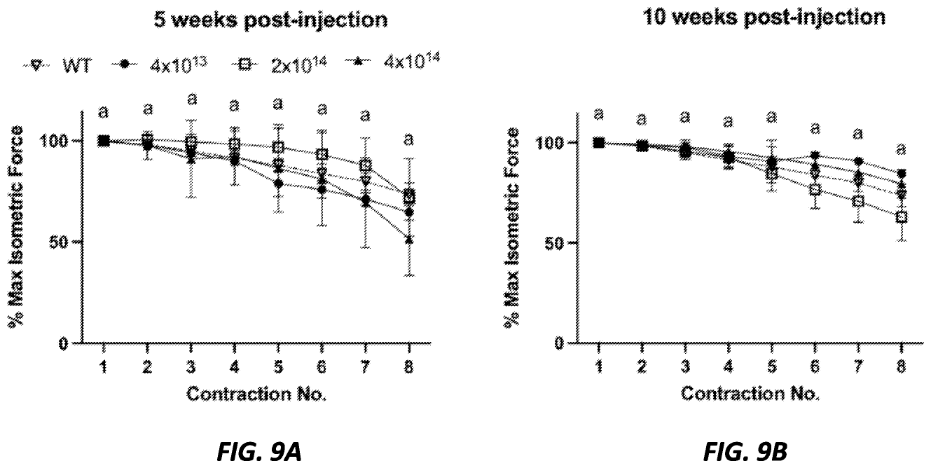

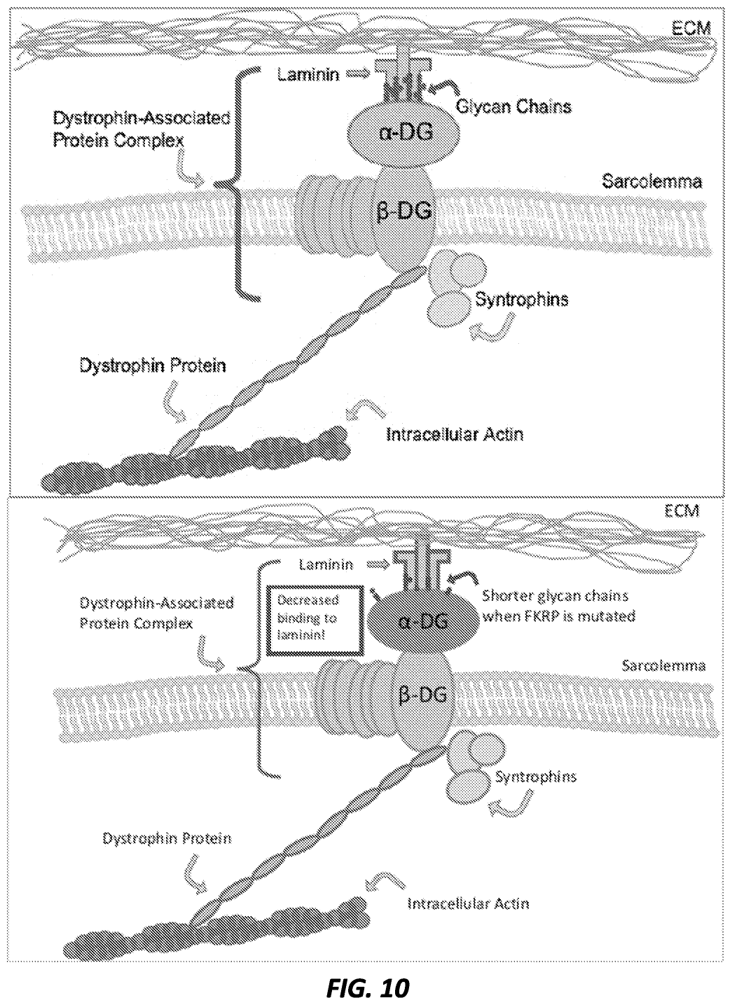

The first study consisted of wild-type mice injected with A6.C8hF at doses of 4×10¹³, 2×10¹⁴, or 4×10¹⁴ vg/kg to determine whether this vector caused an increase in susceptibility of muscles to contraction-induced injury. These high doses were a used in this preliminary safety assessment, as doses at or exceeding 1×10¹⁴ vg/kg have often led to serious adverse events in patients with various neuromuscular disorders. This is important because a high dose AAV-mediated FKRP therapeutic, plus endogenous FKRP, provides the ability to maximize potential expression. Some of these mice underwent gastrocnemius muscle physiology assays 5 weeks later and others were tested at 10 weeks. However, no deleterious impact on mechanical properties were observed at these doses (FIG. 9A-9B).

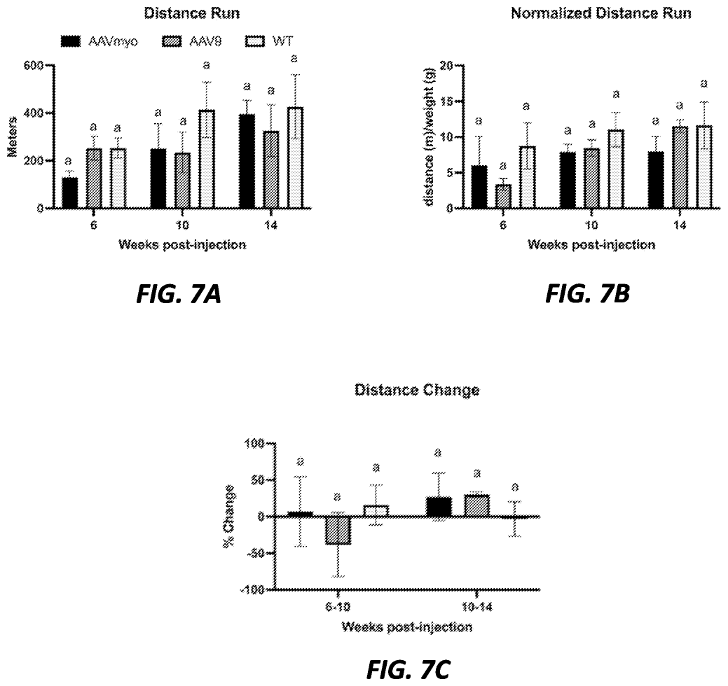

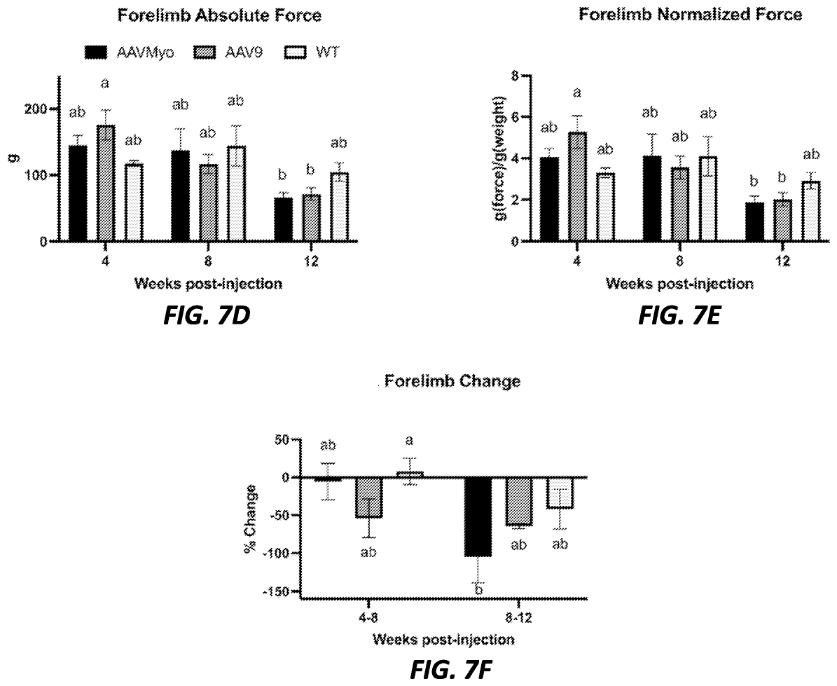

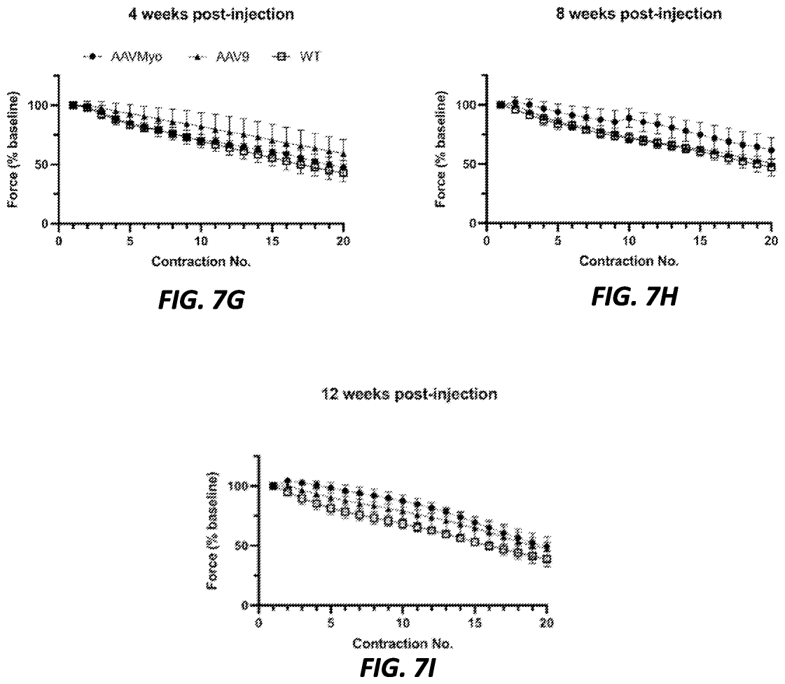

Finally, the AAV9 and AAVMYO1 vectors were injected into wild-type mice at doses of 6.4×10¹², 2×10¹³, and 6.4×10¹³ vg/kg. The latter two cohort of mice were analyzed for a variety of properties at 4-, 6-, 8-, 10-, 12-, and 14-weeks post-injection to evaluate effects on muscle physiology (FIGS. 7A-7I). At 6-, 10-, and 14-weeks post-injection, fatigue assays were performed in which mice ran on a treadmill at a speed of 10 meter/sec, with 1 meter/sec/minute increases (FIG. 7A-7C). No differences were detected between groups at each time point. Moreover, the absence of differences in relative change within and between groups similarly demonstrates an absence of detrimental effects on running fitness and fatiguability following vector delivery. Forelimb grip strength was also examined between 4-, 8-, and 12-weeks post-injection. Although mice treated with AAV9 displayed reduced force over time, there were no differences between groups at any time point. In fact, all the mice (treated and untreated) experienced a decrease in strength between 10- and 14-weeks post-injection groups, which typically occurs when mice habituate to the grip-strength assay.

Another commonly used assay in mouse muscle analysis is the ankle plantarflexion assay, in which ankle torque over 20 eccentric contractions is quantified using a high yet physiologically relevant stimulation frequency that elicits a maximal response. The assay is designed to assess force generation in addition to fatigue caused by repeated eccentric contractions. At all time points, torque declined to 50% of starting baseline measurements, consistent with contraction-induced fatigue (FIG. 7G-7H). However, no differences were detected between any of the three groups at any timepoint. This includes potential differences in torque at a given contraction as well as the relative change from baseline. These results further suggest that A9.C8hF and AM.C8hF treatments at a dose of 6.4×10¹³ vg/kg did not compromise muscle force generation or enhance fatiguability.

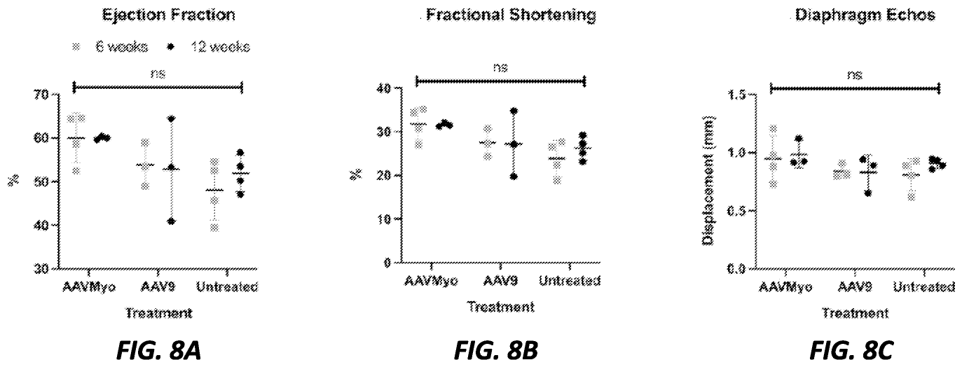

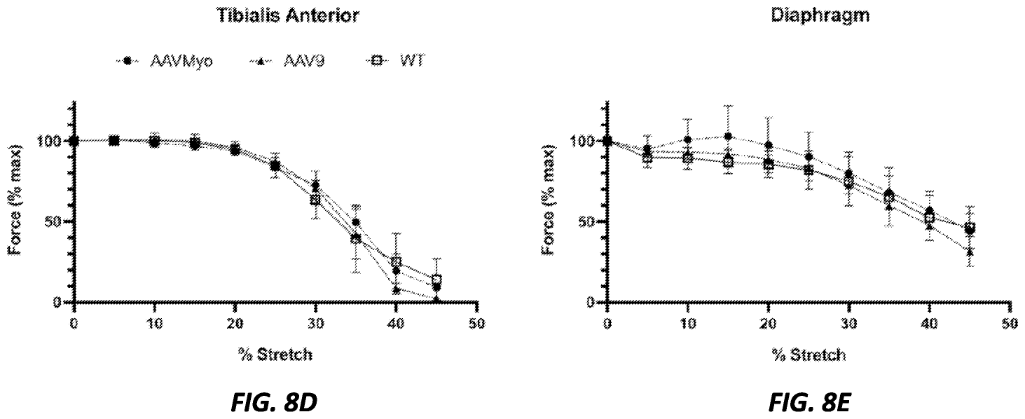

To identify potential adverse consequences of exogenous FKRP expression in cardiac and diaphragm muscles, ultrasound imaging was used to measure cardiac ejection fraction and fractional shortening as well as diaphragm displacement. Ejection fraction measures the percent of blood leaving the left ventricle, while fractional shortening is a measure of the heart's contractility; both of which are common indicators of cardiac function. Diaphragm displacement is likewise an indicator of respiratory function. No differences were detected between untreated and treated groups, likely due to small sample size (n=3-4), however more data may highlight the increased injection fraction of AM.C8hF over A9.C8hF and untreated mice (FIG. 8A-8C). At the experimental endpoint (18 weeks post-injection), skeletal muscle function was further assessed using a contraction-induced injury protocol on tibialis anterior and diaphragm muscles. Here, muscles were stretched by an additional 5% between contractions to measure specific force development and to examine the effects of contraction-induced injury on force. As with the other functional assays, no differences were detected between treated and untreated groups (FIGS. 8D & 8E). These data complement those from previous tests, which together reveal no adverse consequences from exogenous FKRP overexpression in skeletal or cardiac muscles using the assays shown (FIGS. 7 and 8).

### Discussion

This study focused to develop a transgene with high expression levels in striated muscles. Use of such a transgene permits studies of safety while also facilitating therapeutic expression levels at lower vector doses. It should be noted that safety and toxicity concerns have been growing in the field, with adverse events from either the vector or transgene being observed in a variety of high dose, neuromuscular disease AAV trials.

RNA G-quadruplexes (RGQ) are known to regulate gene expression and have been targeted as a strategy to ameliorate or worsen genetic disease pathologies. It was discovered that complete removal of both the 5′ and 3′ UTRs led to increased gene expression (FIG. 6A-6E). These data suggest the presence of inhibitory regulatory sequences within the UTRs of FKRP, which could provide a potential target for fine-tuning gene expression. The occurrence of toxicity associated with gene overexpression has sometimes been resolved by targeting UTRs, which continue to be evolved for even more influence over protein production.

Demonstrating that the therapeutic can improve exercise capacity is especially meaningful, as the metrics measured are largely dependent upon cardiac rather than skeletal muscle function. Moreover, cardiac and respiratory impairment directly contribute to mortality in many individuals with different FKRP mutations and are predictors of long-term survival. It is not unreasonable, therefore, to presume that systemic FKRP gene therapy could ameliorate the primary cause of mortality in LGMDR9 patients.

Safety Assessment with Exogenous FKRP Expression

While studies here and by others show improved phenotypic outcome from AAV-FKRP delivery, the effects in wild-type mice were explored to monitor potential adverse events from exogenous FKRP expression above normal muscle expression. These exogenous expression levels were augmented using a strong, muscle-specific regulatory cassette (CK8e), the removal of UTRs from the FKRP cDNA and the use of myotropic AAV capsids, especially AAVMYO1. The AAVMYO1 capsid in particular is one of a new class of potent muscle-targeting capsids and may allow lower doses to be used in clinical trials. Three doses of 4×10¹³, 2×10¹⁴, and 4×10¹⁴ vg/kg of A6.C8hF indicated that treated wild-type mice continued to gain strength, did not fatigue more rapidly, and showed no detrimental effects on mechanical properties (FIG. 9). A longer study was also performed with wild-type mice untreated or injected with A9.C8hF and AM.C8hF. These groups of mice showed no significant differences in distance run, forelimb grip strength, or TA, diaphragm muscle or hind limb eccentric contraction induced fatigue over time, nor were these measurements significantly different between groups (FIG. 7). In these studies, treated and untreated mice also showed no change in ejection fraction, fractional shortening, nor diaphragm displacement over time (FIG. 8). Overall the data support the safety of A6.Ch8F, A9.C8hF, and AM.Ch8F as gene therapies for LGMDR9, as the assays show that none of these treatments led to adverse effects on wild-type mouse physiology. These results demonstrate that expression from the vectors used was not deleterious to muscle function and was able to ameliorate disease pathology in mutant mice.

### Conclusions and Future Directions

The studies further support the feasibility of gene therapy for disorders resulting from FKRP mutations. Further, no detrimental effects from expression of FKRP were observed using a variety of AAV vectors at different doses in wild-type mice. A potential G-quadruplex in the 5′ UTR may serve to limit expression, although individual bases were dissected in the 5′ or 3′ UTRs of the FKRP mRNA that mediate this effect. The difficulty encountered by many labs in detecting endogenous FKRP expression suggests that extremely low levels of the enzyme is all that is needed for normal glycosylation of «-DG. It is possible that inhibitory UTR sequences may serve a role in limiting overexpression during normal muscle activity, although further studies would be needed to clarify this issue. Importantly, the safety of this therapeutic observed in the studies supports continued advancement of methods for gene therapy to treat patients carrying mutations in the FKRP gene.

### Example 14: Materials and Methods

### Animals

All animal experiments were approved by the Institutional Animal Care and Use Committee of the University of Washington Mouse studies were performed in C57BL6 wild-type male mice aged 6-35 weeks at age of injection.

### Plasmid Construction and Vector Production

The coding region of mouse Fkrp was PCR amplified (Forward primer: 5′ TTGTTAACATGCGGCTCACCC 3′ (SEQ ID NO: 30); Reverse primer: 5′ TACCGGTTCAACCGCCTGTC 3′ (SEQ ID NO: 31)) from mouse muscle cDNA. The resulting DNA fragment was digested with HpaI and AgeI and ligated into an AAV backbone vector containing a muscle specific CK8e promoter and synthetic poly A tail, as previously described. Briefly, a custom AAV transfer plasmid containing the previously described muscle-specific CK8e regulator cassette, a 1,482 bp cDNA expression construct for native mouse Fkrp mRNA (NCBI CCDS20853.1), a synthetic polyA signal and flanking AAV serotype 2 inverted terminal repeats was constructed using standard recombinant methodology. The resulting plasmid, pAAV-CK8e-FKRP, was then used to co-transfect HEK293 cells along with the pDGM6 helper plasmid to generate AAV6 vector as previously described. For AAV9 (pA9.C8hF) and AAVMYO1 (AM.C8hF) vectors the human FKRP cDNA was PCR amplified and cloned into pAAV-Ck8e-FKRP in place of the mouse cDNA. These latter vector preparations were produced and purified by Forge Biologics (Grove City, OH).

### Antibody Production and Purification

Rabbit polyclonal antisera was generated against a peptide near the C-terminus of the FKRP sequence that was identical in the mouse and human proteins (as a fusion with KLH: KLH-C-APNNYRRFLELKFGPGVIENPQYPNP (SEQ ID NO: 32)) by Covance (Denver, PA). Affinity purification was performed using a maltose-binding protein fusion of the antigenic peptide on a MBPTrap HP columns (GE Healthcare) and coupled to Ultralink Biosupport polyacrylamide resin (Thermo Scientific) as per manufacturer's instructions. The FKRP-C antibody (named Ab607) was affinity purified by HPLC through MBP-FKRP coupled beads and stored in BSA and NaN₃.

### Cell Culture

Mouse C2C12 cells were plated at ˜80% confluence on gelatin-coated 6-well plates with standard growth media (DMEM, 20% FBS, 1% penicillin-streptomycin (P/S)) overnight, then washed three times with 1× Saline G prior to infection with rAAV6-CK8-mFKRP. Vectors were diluted to the desired concentrations in differentiation media (DMEM, 2% HS, 1% Penicillin-Streptomycin). Cells in each well were incubated with 0.5 ml of diluted virus at 37° C. for ˜ 2 hours and brought to a final volume of 2 ml with differentiation media and incubated overnight prior to refeeding with fresh differentiation media. Western analysis was performed at day 3 post-differentiation to determine FKRP expression.

### Gene Delivery

Mice were randomly assigned to groups prior to treatment. They were then anesthetized by an intraperitoneal injection of 0.25 mg/g 2,2,2-tribromoethanol or isoflurane and subsequently injected retro-orbitally (RO) (150 μl at doses of 1.5×10¹³, 4×10¹³, 1.5×10¹⁴, 2×10¹⁴, or 4×10¹⁴ vg/kg), intramuscularly (30 μl at doses of 1×10¹⁰ vg and 1×10¹¹ vg), or intravenously via the tail vein (150 μl at doses of 6.4×10¹³ vg/kg).

### Western Analysis

Frozen muscles were ground to fine powder and proteins extracted in 1× Laemmli buffer (62.5 mM Tris-HCl, pH 6.8, 2% SDS, 10% glycerol, protease inhibitor (Roche)). Lysates were centrifuged at 10,000×g for 10 min at 4° C., and supernatants transferred to fresh tubes. Total protein concentration was determined by BCA Assay (Pierce). Prior to loading, 50 mM DTT and 0.01% bromophenol blue were added to samples and heated at 94° C. for 4 min. For each sample, 20 μg of total protein was separated on 4-12% SDS-PAGE (Life Technologies), transferred to PVDF membranes (GE Healthcare), and blocked (5% skim milk, 1×PBS, 0.1% Tween-20) for 1 hr at room temperature. Blots were probed with Ab607 (1:2000) overnight at 4° C., washed in 1×PBS-Tween, incubated with HRP-Rabbit secondary (1:20,000, Pierce) and developed with ECL chemiluminescent reagents (GE Healthcare).

### Statistical Analysis

Data are presented as means±SEM and statistical comparisons of non-parametric data points were made using Prism (GraphPad Software, La Jolla, CA). Significant differences (p≤0.05 unless otherwise noted) were determined using a Student's t-test or with a 1- or 2-way analysis of variance coupled to Tukey's post-hoc test for multiple mean comparisons. Significant differences are represented by different letters and a shared letter indicates no difference. For example, three data points labelled a, ab, and b represent a significant difference between a and b, as different letters indicate significance. There is no difference between a and ab or ab and b, as they share letters.

### Example 16

Another way to analyze and verify the effects of gene therapy with FKRP constructs as described herein is to administer vectors as described herein to a mouse model of FKRP deficiency-mediated disease. To that end, FKRP^{P448L} mice (which contain a mutation in the FKRP ORF at position 448 (P448L) between the ages of 6 months to a year (the preferred age is 10 months) can be injected with different doses of A6.C8hF (saline, at a range between 1×10¹², and 1×10¹⁵ vg/kg) and monitored alongside age-matched wild-type controls. The purpose of using older mice is to address the effect of treatment in older mice with a more advanced phenotype. Forelimb grip strength of age from wild-type, untreated and treated FKRP^{P448L} mice can be measured between 2-weeks and 16-weeks post-injection. It is expected that absolute forelimb grip strength will restore strength, e.g., between 30%-80% of wild-type levels or more, compared to untreated FKRP^{P448L} mice. Additionally, it is expected that body weight and grip strength will increase modestly in all treated FKRP^{P448L} mice. Such a change would indicate that changes in grip strength are unlikely to be due to compensatory responses to changes in body weight.

Hindlimb grip-strength measurements are often highly variable until mice become accustomed to the assay. Based on this, it can be expected that grip-strength absolute force will increase in all groups before stabilizing between 7-10 weeks. However, it is also predicted that treated mice will also be stronger than untreated. These results will suggest that despite the innate variability of the assay, treatment with constructs as described herein, including but not limited to A6.C8hF, will increase hindlimb grip strength in aged mice.

### Example 17

Exercise capacity of mice treated with FKRP viral vectors as described herein. Additional metrics that can be examined in treated mutant mice include measurements of VO₂max, distance travelled, energy expended, and energy consumption rate. All of these can be measured using a metabolic treadmill and can include VO₂max tests to assess changes in maximal 02 consumption, a measure of exercise capacity and cardiorespiratory function, and several intermittent training sessions meant to exacerbate the dystrophic phenotype. It is expected that training will have a beneficial effect in wild-type mice and will increase VO₂max between 5%-15%. By contrast, it is expected that training will exacerbate the dystrophic phenotype in untreated FKRP^{P448L} mice as VO₂max will be reduced by over 5%-20%. It is expected that this decline will be prevented by A6.C8hF treatment. Moreover, it is expected that the degree of relative change between tests will be significant among all three groups, indicating the effect of impact training and treatment. It is expected that changes in running distances and energy rates will reflect a similar relationship. These results will collectively suggest that although high dose treatment with A6.C8hF may not restore exercise capacity and cardiorespiratory function to wild-type levels, it prevents the deleterious effects of impact training and improves striated muscle functional efficiency. It should be noted that FKRP mutations in both animal models and human patients leads to a degenerative disease that progressively develops and worsens with age. As such, it would not be unexpected that even successful therapy in a model with advanced disease does not restore function to pre- or non-disease levels. Higher doses or ancillary treatments that enhance muscle mass and strength may be needed when degeneration is advanced.

It is expected that both groups of mice will run shorter distances in the initial tests while expending similar calories. Although this will result in higher energy-consumption rates, these differences are not expected to be significantly different from wild-type mice. It is expected that the protective effect on exercise-induced impact will also be reflected in energy consumption rates. It is expected that all three groups will be significantly different from one another in the final test, with untreated FKRP^{P448L} mice having the lowest VO₂max and highest energy consumption rate. Treated FKRP^{P448L} mice will display values closer to those of wild-type animals. It is expected that treatment with A6.C8hF will be able to at least partially restore exercise capacity.

### Example 18

Effect of FKRP viral vectors as described herein on dystrophic respiratory pattern and endurance exercise capacity in FKRP^{P448L} mice. Several pathological markers can be used for assessing exercise impact training for dystrophic mice. In addition to histological metrics, these include several exercise-induced markers in FKRP^{P448L} mice such as variability in respiration (VO₂cv), the respiratory exchange ratio (RER, VCO₂/VO₂), energy expended, and the accumulation of motivational shocks. It is expected that in some of training sessions, the VO₂cv values for untreated FKRP^{P448L} mice will be higher than those for wild-type mice, while the treated FKRP^{P448L} animals will have values that will be either significantly lower than the untreated mice or not different from wild-type mice. A similar pattern is expected when comparing the overall differences between groups, which would be highly significant, and when assessing differences in maximal RER values.

### Example 19

Evaluation of muscle degeneration, muscle fiber size distribution and creatine kinase levels. After assessing exercise impact training, all mice can be sacrificed, and different muscles will be collected. Compared to untreated FKRP^{P448L} mice, it is expected that treated FKRP^{P448L} mice will have larger skeletal muscle myofiber sizes and will have fewer centrally-nucleated myofibers and fewer total centrally-located nuclei, which are hallmarks of prior rounds of muscle necrosis and regeneration. It is expected that treatment will also impact serum creatine kinase levels in treated vs. untreated mice, with levels in treated mice being significantly lower than in untreated. Such a result would suggest that treatment with FKRP vectors as described herein could reduce exercise-induced muscle damage that activates cycles of necrosis and regeneration that lead to skeletal muscle atrophy.

### Example 20

Combinations of G-quadruplex, hairpin, and 3′+MSEC below based on the description from FIG. 5. The term “FKRP ORF” refers to both wild-type FKRP and a mutated form of FKRP. The term “MSEC” refers to any MSEC as defined in the specification and referenced in PCT/US22/023915, which is incorporated by reference in its entirety. The following describes various combinations of 5′ UTR and 3′ UTR modifications in combination with MSECs contemplated for FKRP constructs as described herein.

### 5′ UTR Truncations

−01 bp to −143 bp from 5′ UTR end+FKRP ORF+3′ UTR+MSEC
−02 bp to −143 bp from 5′ UTR end+FKRP ORF+3′ UTR+MSEC
−03 bp to −143 bp from 5′ UTR end+FKRP ORF+3′ UTR+MSEC
−04 bp to −143 bp from 5′ UTR end+FKRP ORF+3′ UTR+MSEC
−05 bp to −143 bp from 5′ UTR end+FKRP ORF+3′ UTR+MSEC
−06 bp to −143 bp from 5′ UTR end+FKRP ORF+3′ UTR+MSEC
−07 bp to −143 bp from 5′ UTR end+FKRP ORF+3′ UTR+MSEC
−08 bp to −143 bp from 5′ UTR end+FKRP ORF+3′ UTR+MSEC
−09 bp to −143 bp from 5′ UTR end+FKRP ORF+3′ UTR+MSEC
−10 bp to −143 bp from 5′ UTR end+FKRP ORF+3′ UTR+MSEC
−11 bp to −143 bp from 5′ UTR end+FKRP ORF+3′ UTR+MSEC
−12 bp to −143 bp from 5′ UTR end+FKRP ORF+3′ UTR+MSEC
−13 bp to −143 bp from 5′ UTR end+FKRP ORF+3′ UTR+MSEC
−14 bp to −143 bp from 5′ UTR end+FKRP ORF+3′ UTR+MSEC
−15 bp to −143 bp from 5′ UTR end+FKRP ORF+3′ UTR+MSEC
−16 bp to −143 bp from 5′ UTR end+FKRP ORF+3′ UTR+MSEC
−17 bp to −143 bp from 5′ UTR end+FKRP ORF+3′ UTR+MSEC
−18 bp to −143 bp from 5′ UTR end+FKRP ORF+3′ UTR+MSEC
−19 bp to −143 bp from 5′ UTR end+FKRP ORF+3′ UTR+MSEC
−20 bp to −143 bp from 5′ UTR end+FKRP ORF+3′ UTR+MSEC
−21 bp to −143 bp from 5′ UTR end+FKRP ORF+3′ UTR+MSEC
−22 bp to −143 bp from 5′ UTR end+FKRP ORF+3′ UTR+MSEC
−23 bp to −143 bp from 5′ UTR end+FKRP ORF+3′ UTR+MSEC
−24 bp to −143 bp from 5′ UTR end+FKRP ORF+3′ UTR+MSEC
−25 bp to −143 bp from 5′ UTR end+FKRP ORF+3′ UTR+MSEC
−26 bp to −143 bp from 5′ UTR end+FKRP ORF+3′ UTR+MSEC
−27 bp to −143 bp from 5′ UTR end+FKRP ORF+3′ UTR+MSEC
−28 bp to −143 bp from 5′ UTR end+FKRP ORF+3′ UTR+MSEC
−29 bp to −143 bp from 5′ UTR end+FKRP ORF+3′ UTR+MSEC
−30 bp to −143 bp from 5′ UTR end+FKRP ORF+3′ UTR+MSEC
−31 bp to −143 bp from 5′ UTR end+FKRP ORF+3′ UTR+MSEC
−32 bp to −143 bp from 5′ UTR end+FKRP ORF+3′ UTR+MSEC
−33 bp to −143 bp from 5′ UTR end+FKRP ORF+3′ UTR+MSEC
−34 bp to −143 bp from 5′ UTR end+FKRP ORF+3′ UTR+MSEC
−35 bp to −143 bp from 5′ UTR end+FKRP ORF+3′ UTR+MSEC
−36 bp to −143 bp from 5′ UTR end+FKRP ORF+3′ UTR+MSEC
−37 bp to −143 bp from 5′ UTR end+FKRP ORF+3′ UTR+MSEC
−38 bp to −143 bp from 5′ UTR end+FKRP ORF+3′ UTR+MSEC
−39 bp to −143 bp from 5′ UTR end+FKRP ORF+3′ UTR+MSEC
−40 bp to −143 bp from 5′ UTR end+FKRP ORF+3′ UTR+MSEC
−41 bp to −143 bp from 5′ UTR end+FKRP ORF+3′ UTR+MSEC
−42 bp to −143 bp from 5′ UTR end+FKRP ORF+3′ UTR+MSEC
−43 bp to −143 bp from 5′ UTR end+FKRP ORF+3′ UTR+MSEC
−44 bp to −143 bp from 5′ UTR end+FKRP ORF+3′ UTR+MSEC
−45 bp to −143 bp from 5′ UTR end+FKRP ORF+3′ UTR+MSEC
−46 bp to −143 bp from 5′ UTR end+FKRP ORF+3′ UTR+MSEC
−47 bp to −143 bp from 5′ UTR end+FKRP ORF+3′ UTR+MSEC
−48 bp to −143 bp from 5′ UTR end+FKRP ORF+3′ UTR+MSEC
−49 bp to −143 bp from 5′ UTR end+FKRP ORF+3′ UTR+MSEC
−50 bp to −143 bp from 5′ UTR end+FKRP ORF+3′ UTR+MSEC
−51 bp to −143 bp from 5′ UTR end+FKRP ORF+3′ UTR+MSEC
−52 bp to −143 bp from 5′ UTR end+FKRP ORF+3′ UTR+MSEC
−53 bp to −143 bp from 5′ UTR end+FKRP ORF+3′ UTR+MSEC
−54 bp to −143 bp from 5′ UTR end+FKRP ORF+3′ UTR+MSEC
−55 bp to −143 bp from 5′ UTR end+FKRP ORF+3′ UTR+MSEC
−56 bp to −143 bp from 5′ UTR end+FKRP ORF+3′ UTR+MSEC
−57 bp to −143 bp from 5′ UTR end+FKRP ORF+3′ UTR+MSEC
−58 bp to −143 bp from 5′ UTR end+FKRP ORF+3′ UTR+MSEC
−59 bp to −143 bp from 5′ UTR end+FKRP ORF+3′ UTR+MSEC
−60 bp to −143 bp from 5′ UTR end+FKRP ORF+3′ UTR+MSEC
−61 bp to −143 bp from 5′ UTR end+FKRP ORF+3′ UTR+MSEC
−62 bp to −143 bp from 5′ UTR end+FKRP ORF+3′ UTR+MSEC
−63 bp to −143 bp from 5′ UTR end+FKRP ORF+3′ UTR+MSEC
−64 bp to −143 bp from 5′ UTR end+FKRP ORF+3′ UTR+MSEC
−65 bp to −143 bp from 5′ UTR end+FKRP ORF+3′ UTR+MSEC
−66 bp to −143 bp from 5′ UTR end+FKRP ORF+3′ UTR+MSEC
−67 bp to −143 bp from 5′ UTR end+FKRP ORF+3′ UTR+MSEC
−68 bp to −143 bp from 5′ UTR end+FKRP ORF+3′ UTR+MSEC
−69 bp to −143 bp from 5′ UTR end+FKRP ORF+3′ UTR+MSEC
−70 bp to −143 bp from 5′ UTR end+FKRP ORF+3′ UTR+MSEC
−71 bp to −143 bp from 5′ UTR end+FKRP ORF+3′ UTR+MSEC
−72 bp to −143 bp from 5′ UTR end+FKRP ORF+3′ UTR+MSEC
−73 bp to −143 bp from 5′ UTR end+FKRP ORF+3′ UTR+MSEC
−74 bp to −143 bp from 5′ UTR end+FKRP ORF+3′ UTR+MSEC
−75 bp to −143 bp from 5′ UTR end+FKRP ORF+3′ UTR+MSEC
−76 bp to −143 bp from 5′ UTR end+FKRP ORF+3′ UTR+MSEC
−77 bp to −143 bp from 5′ UTR end+FKRP ORF+3′ UTR+MSEC
−78 bp to −143 bp from 5′ UTR end+FKRP ORF+3′ UTR+MSEC
−79 bp to −143 bp from 5′ UTR end+FKRP ORF+3′ UTR+MSEC
−80 bp to −143 bp from 5′ UTR end+FKRP ORF+3′ UTR+MSEC
−81 bp to −143 bp from 5′ UTR end+FKRP ORF+3′ UTR+MSEC
−82 bp to −143 bp from 5′ UTR end+FKRP ORF+3′ UTR+MSEC
−83 bp to −143 bp from 5′ UTR end+FKRP ORF+3′ UTR+MSEC
−84 bp to −143 bp from 5′ UTR end+FKRP ORF+3′ UTR+MSEC
−85 bp to −143 bp from 5′ UTR end+FKRP ORF+3′ UTR+MSEC
−86 bp to −143 bp from 5′ UTR end+FKRP ORF+3′ UTR+MSEC
−87 bp to −143 bp from 5′ UTR end+FKRP ORF+3′ UTR+MSEC
−88 bp to −143 bp from 5′ UTR end+FKRP ORF+3′ UTR+MSEC
−89 bp to −143 bp from 5′ UTR end+FKRP ORF+3′ UTR+MSEC
−90 bp to −143 bp from 5′ UTR end+FKRP ORF+3′ UTR+MSEC
−91 bp to −143 bp from 5′ UTR end+FKRP ORF+3′ UTR+MSEC
−92 bp to −143 bp from 5′ UTR end+FKRP ORF+3′ UTR+MSEC
−93 bp to −143 bp from 5′ UTR end+FKRP ORF+3′ UTR+MSEC
−94 bp to −143 bp from 5′ UTR end+FKRP ORF+3′ UTR+MSEC
−95 bp to −143 bp from 5′ UTR end+FKRP ORF+3′ UTR+MSEC
−96 bp to −143 bp from 5′ UTR end+FKRP ORF+3′ UTR+MSEC
−97 bp to −143 bp from 5′ UTR end+FKRP ORF+3′ UTR+MSEC
−98 bp to −143 bp from 5′ UTR end+FKRP ORF+3′ UTR+MSEC
−99 bp to −143 bp from 5′ UTR end+FKRP ORF+3′ UTR+MSEC
−100 bp to −143 bp from 5′ UTR end+FKRP ORF+3′ UTR+MSEC
−101 bp to −143 bp from 5′ UTR end+FKRP ORF+3′ UTR+MSEC
−102 bp to −143 bp from 5′ UTR end+FKRP ORF+3′ UTR+MSEC
−103 bp to −143 bp from 5′ UTR end+FKRP ORF+3′ UTR+MSEC
−104 bp to −143 bp from 5′ UTR end+FKRP ORF+3′ UTR+MSEC
−105 bp to −143 bp from 5′ UTR end+FKRP ORF+3′ UTR+MSEC
−106 bp to −143 bp from 5′ UTR end+FKRP ORF+3′ UTR+MSEC
−107 bp to −143 bp from 5′ UTR end+FKRP ORF+3′ UTR+MSEC
−108 bp to −143 bp from 5′ UTR end+FKRP ORF+3′ UTR+MSEC
−109 bp to −143 bp from 5′ UTR end+FKRP ORF+3′ UTR+MSEC
−110 bp to −143 bp from 5′ UTR end+FKRP ORF+3′ UTR+MSEC
−111 bp to −143 bp from 5′ UTR end+FKRP ORF+3′ UTR+MSEC
−112 bp to −143 bp from 5′ UTR end+FKRP ORF+3′ UTR+MSEC
−113 bp to −143 bp from 5′ UTR end+FKRP ORF+3′ UTR+MSEC
−114 bp to −143 bp from 5′ UTR end+FKRP ORF+3′ UTR+MSEC
−115 bp to −143 bp from 5′ UTR end+FKRP ORF+3′ UTR+MSEC
−116 bp to −143 bp from 5′ UTR end+FKRP ORF+3′ UTR+MSEC
−117 bp to −143 bp from 5′ UTR end+FKRP ORF+3′ UTR+MSEC
−118 bp to −143 bp from 5′ UTR end+FKRP ORF+3′ UTR+MSEC
−119 bp to −143 bp from 5′ UTR end+FKRP ORF+3′ UTR+MSEC
−120 bp to −143 bp from 5′ UTR end+FKRP ORF+3′ UTR+MSEC
−121 bp to −143 bp from 5′ UTR end+FKRP ORF+3′ UTR+MSEC
−122 bp to −143 bp from 5′ UTR end+FKRP ORF+3′ UTR+MSEC
−123 bp to −143 bp from 5′ UTR end+FKRP ORF+3′ UTR+MSEC
−124 bp to −143 bp from 5′ UTR end+FKRP ORF+3′ UTR+MSEC
−125 bp to −143 bp from 5′ UTR end+FKRP ORF+3′ UTR+MSEC
−126 bp to −143 bp from 5′ UTR end+FKRP ORF+3′ UTR+MSEC
−127 bp to −143 bp from 5′ UTR end+FKRP ORF+3′ UTR+MSEC
−128 bp to −143 bp from 5′ UTR end+FKRP ORF+3′ UTR+MSEC
−129 bp to −143 bp from 5′ UTR end+FKRP ORF+3′ UTR+MSEC
−130 bp to −143 bp from 5′ UTR end+FKRP ORF+3′ UTR+MSEC
−131 bp to −143 bp from 5′ UTR end+FKRP ORF+3′ UTR+MSEC
−132 bp to −143 bp from 5′ UTR end+FKRP ORF+3′ UTR+MSEC
−133 bp to −143 bp from 5′ UTR end+FKRP ORF+3′ UTR+MSEC
−134 bp to −143 bp from 5′ UTR end+FKRP ORF+3′ UTR+MSEC
−135 bp to −143 bp from 5′ UTR end+FKRP ORF+3′ UTR+MSEC
−136 bp to −143 bp from 5′ UTR end+FKRP ORF+3′ UTR+MSEC
−137 bp to −143 bp from 5′ UTR end+FKRP ORF+3′ UTR+MSEC
−138 bp to −143 bp from 5′ UTR end+FKRP ORF+3′ UTR+MSEC
−139 bp to −143 bp from 5′ UTR end+FKRP ORF+3′ UTR+MSEC
−140 bp to −143 bp from 5′ UTR end+FKRP ORF+3′ UTR+MSEC
−141 bp to −143 bp from 5′ UTR end+FKRP ORF+3′ UTR+MSEC
−142 bp to −143 bp from 5′ UTR end+FKRP ORF+3′ UTR+MSEC
+1 bp to −142 bp from 5′ UTR end+FKRP ORF+3′ UTR+MSEC
+1 bp to −141 bp from 5′ UTR end+FKRP ORF+3′ UTR+MSEC
+1 bp to −140 bp from 5′ UTR end+FKRP ORF+3′ UTR+MSEC
+1 bp to −139 bp from 5′ UTR end+FKRP ORF+3′ UTR+MSEC
+1 bp to −138 bp from 5′ UTR end+FKRP ORF+3′ UTR+MSEC
+1 bp to −137 bp from 5′ UTR end+FKRP ORF+3′ UTR+MSEC
+1 bp to −136 bp from 5′ UTR end+FKRP ORF+3′ UTR+MSEC
+1 bp to −135 bp from 5′ UTR end+FKRP ORF+3′ UTR+MSEC
+1 bp to −134 bp from 5′ UTR end+FKRP ORF+3′ UTR+MSEC
+1 bp to −133 bp from 5′ UTR end+FKRP ORF+3′ UTR+MSEC
+1 bp to −132 bp from 5′ UTR end+FKRP ORF+3′ UTR+MSEC
+1 bp to −131 bp from 5′ UTR end+FKRP ORF+3′ UTR+MSEC
+1 bp to −130 bp from 5′ UTR end+FKRP ORF+3′ UTR+MSEC
+1 bp to −129 bp from 5′ UTR end+FKRP ORF+3′ UTR+MSEC
+1 bp to −128 bp from 5′ UTR end+FKRP ORF+3′ UTR+MSEC
+1 bp to −127 bp from 5′ UTR end+FKRP ORF+3′ UTR+MSEC
+1 bp to −126 bp from 5′ UTR end+FKRP ORF+3′ UTR+MSEC
+1 bp to −125 bp from 5′ UTR end+FKRP ORF+3′ UTR+MSEC
+1 bp to −124 bp from 5′ UTR end+FKRP ORF+3′ UTR+MSEC
+1 bp to −123 bp from 5′ UTR end+FKRP ORF+3′ UTR+MSEC
+1 bp to −122 bp from 5′ UTR end+FKRP ORF+3′ UTR+MSEC
+1 bp to −121 bp from 5′ UTR end+FKRP ORF+3′ UTR+MSEC
+1 bp to −120 bp from 5′ UTR end+FKRP ORF+3′ UTR+MSEC
+1 bp to −119 bp from 5′ UTR end+FKRP ORF+3′ UTR+MSEC
+1 bp to −118 bp from 5′ UTR end+FKRP ORF+3′ UTR+MSEC
+1 bp to −117 bp from 5′ UTR end+FKRP ORF+3′ UTR+MSEC
+1 bp to −116 bp from 5′ UTR end+FKRP ORF+3′ UTR+MSEC
+1 bp to −115 bp from 5′ UTR end+FKRP ORF+3′ UTR+MSEC
+1 bp to −114 bp from 5′ UTR end+FKRP ORF+3′ UTR+MSEC
+1 bp to −113 bp from 5′ UTR end+FKRP ORF+3′ UTR+MSEC
+1 bp to −112 bp from 5′ UTR end+FKRP ORF+3′ UTR+MSEC
+1 bp to −111 bp from 5′ UTR end+FKRP ORF+3′ UTR+MSEC
+1 bp to −110 bp from 5′ UTR end+FKRP ORF+3′ UTR+MSEC
+1 bp to −109 bp from 5′ UTR end+FKRP ORF+3′ UTR+MSEC
+1 bp to −108 bp from 5′ UTR end+FKRP ORF+3′ UTR+MSEC
+1 bp to −107 bp from 5′ UTR end+FKRP ORF+3′ UTR+MSEC
+1 bp to −106 bp from 5′ UTR end+FKRP ORF+3′ UTR+MSEC
+1 bp to −105 bp from 5′ UTR end+FKRP ORF+3′ UTR+MSEC
+1 bp to −104 bp from 5′ UTR end+FKRP ORF+3′ UTR+MSEC
+1 bp to −103 bp from 5′ UTR end+FKRP ORF+3′ UTR+MSEC
+1 bp to −102 bp from 5′ UTR end+FKRP ORF+3′ UTR+MSEC
+1 bp to −101 bp from 5′ UTR end+FKRP ORF+3′ UTR+MSEC
+1 bp to −100 bp from 5′ UTR end+FKRP ORF+3′ UTR+MSEC
+1 bp to −99 bp from 5′ UTR end+FKRP ORF+3′ UTR+MSEC
+1 bp to −98 bp from 5′ UTR end+FKRP ORF+3′ UTR+MSEC
+1 bp to −97 bp from 5′ UTR end+FKRP ORF+3′ UTR+MSEC
+1 bp to −96 bp from 5′ UTR end+FKRP ORF+3′ UTR+MSEC
+1 bp to −95 bp from 5′ UTR end+FKRP ORF+3′ UTR+MSEC
+1 bp to −94 bp from 5′ UTR end+FKRP ORF+3′ UTR+MSEC
+1 bp to −93 bp from 5′ UTR end+FKRP ORF+3′ UTR+MSEC
+1 bp to −92 bp from 5′ UTR end+FKRP ORF+3′ UTR+MSEC
+1 bp to −91 bp from 5′ UTR end+FKRP ORF+3′ UTR+MSEC
+1 bp to −90 bp from 5′ UTR end+FKRP ORF+3′ UTR+MSEC
+1 bp to −89 bp from 5′ UTR end+FKRP ORF+3′ UTR+MSEC
+1 bp to −88 bp from 5′ UTR end+FKRP ORF+3′ UTR+MSEC
+1 bp to −87 bp from 5′ UTR end+FKRP ORF+3′ UTR+MSEC
+1 bp to −86 bp from 5′ UTR end+FKRP ORF+3′ UTR+MSEC
+1 bp to −85 bp from 5′ UTR end+FKRP ORF+3′ UTR+MSEC
+1 bp to −84 bp from 5′ UTR end+FKRP ORF+3′ UTR+MSEC
+1 bp to −83 bp from 5′ UTR end+FKRP ORF+3′ UTR+MSEC
+1 bp to −82 bp from 5′ UTR end+FKRP ORF+3′ UTR+MSEC
+1 bp to −81 bp from 5′ UTR end+FKRP ORF+3′ UTR+MSEC
+1 bp to −80 bp from 5′ UTR end+FKRP ORF+3′ UTR+MSEC
+1 bp to −79 bp from 5′ UTR end+FKRP ORF+3′ UTR+MSEC
+1 bp to −78 bp from 5′ UTR end+FKRP ORF+3′ UTR+MSEC
+1 bp to −77 bp from 5′ UTR end+FKRP ORF+3′ UTR+MSEC
+1 bp to −76 bp from 5′ UTR end+FKRP ORF+3′ UTR+MSEC
+1 bp to −75 bp from 5′ UTR end+FKRP ORF+3′ UTR+MSEC
+1 bp to −74 bp from 5′ UTR end+FKRP ORF+3′ UTR+MSEC
+1 bp to −73 bp from 5′ UTR end+FKRP ORF+3′ UTR+MSEC
+1 bp to −72 bp from 5′ UTR end+FKRP ORF+3′ UTR+MSEC
+1 bp to −71 bp from 5′ UTR end+FKRP ORF+3′ UTR+MSEC
+1 bp to −70 bp from 5′ UTR end+FKRP ORF+3′ UTR+MSEC
+1 bp to −69 bp from 5′ UTR end+FKRP ORF+3′ UTR+MSEC
+1 bp to −68 bp from 5′ UTR end+FKRP ORF+3′ UTR+MSEC
+1 bp to −67 bp from 5′ UTR end+FKRP ORF+3′ UTR+MSEC
+1 bp to −66 bp from 5′ UTR end+FKRP ORF+3′ UTR+MSEC
+1 bp to −65 bp from 5′ UTR end+FKRP ORF+3′ UTR+MSEC
+1 bp to −64 bp from 5′ UTR end+FKRP ORF+3′ UTR+MSEC
+1 bp to −63 bp from 5′ UTR end+FKRP ORF+3′ UTR+MSEC
+1 bp to −62 bp from 5′ UTR end+FKRP ORF+3′ UTR+MSEC
+1 bp to −61 bp from 5′ UTR end+FKRP ORF+3′ UTR+MSEC
+1 bp to −60 bp from 5′ UTR end+FKRP ORF+3′ UTR+MSEC
+1 bp to −59 bp from 5′ UTR end+FKRP ORF+3′ UTR+MSEC
+1 bp to −58 bp from 5′ UTR end+FKRP ORF+3′ UTR+MSEC
+1 bp to −57 bp from 5′ UTR end+FKRP ORF+3′ UTR+MSEC
+1 bp to −56 bp from 5′ UTR end+FKRP ORF+3′ UTR+MSEC
+1 bp to −55 bp from 5′ UTR end+FKRP ORF+3′ UTR+MSEC
+1 bp to −54 bp from 5′ UTR end+FKRP ORF+3′ UTR+MSEC
+1 bp to −53 bp from 5′ UTR end+FKRP ORF+3′ UTR+MSEC
+1 bp to −52 bp from 5′ UTR end+FKRP ORF+3′ UTR+MSEC
+1 bp to −51 bp from 5′ UTR end+FKRP ORF+3′ UTR+MSEC
+1 bp to −50 bp from 5′ UTR end+FKRP ORF+3′ UTR+MSEC
+1 bp to −49 bp from 5′ UTR end+FKRP ORF+3′ UTR+MSEC
+1 bp to −48 bp from 5′ UTR end+FKRP ORF+3′ UTR+MSEC
+1 bp to −47 bp from 5′ UTR end+FKRP ORF+3′ UTR+MSEC
+1 bp to −46 bp from 5′ UTR end+FKRP ORF+3′ UTR+MSEC
+1 bp to −45 bp from 5′ UTR end+FKRP ORF+3′ UTR+MSEC
+1 bp to −44 bp from 5′ UTR end+FKRP ORF+3′ UTR+MSEC
+1 bp to −43 bp from 5′ UTR end+FKRP ORF+3′ UTR+MSEC
+1 bp to −42 bp from 5′ UTR end+FKRP ORF+3′ UTR+MSEC
+1 bp to −41 bp from 5′ UTR end+FKRP ORF+3′ UTR+MSEC
+1 bp to −40 bp from 5′ UTR end+FKRP ORF+3′ UTR+MSEC
+1 bp to −39 bp from 5′ UTR end+FKRP ORF+3′ UTR+MSEC
+1 bp to −38 bp from 5′ UTR end+FKRP ORF+3′ UTR+MSEC
+1 bp to −37 bp from 5′ UTR end+FKRP ORF+3′ UTR+MSEC
+1 bp to −36 bp from 5′ UTR end+FKRP ORF+3′ UTR+MSEC
+1 bp to −35 bp from 5′ UTR end+FKRP ORF+3′ UTR+MSEC
+1 bp to −34 bp from 5′ UTR end+FKRP ORF+3′ UTR+MSEC
+1 bp to −33 bp from 5′ UTR end+FKRP ORF+3′ UTR+MSEC
+1 bp to −32 bp from 5′ UTR end+FKRP ORF+3′ UTR+MSEC
+1 bp to −31 bp from 5′ UTR end+FKRP ORF+3′ UTR+MSEC
+1 bp to −30 bp from 5′ UTR end+FKRP ORF+3′ UTR+MSEC
+1 bp to −29 bp from 5′ UTR end+FKRP ORF+3′ UTR+MSEC
+1 bp to −28 bp from 5′ UTR end+FKRP ORF+3′ UTR+MSEC
+1 bp to −27 bp from 5′ UTR end+FKRP ORF+3′ UTR+MSEC
+1 bp to −26 bp from 5′ UTR end+FKRP ORF+3′ UTR+MSEC
+1 bp to −25 bp from 5′ UTR end+FKRP ORF+3′ UTR+MSEC
+1 bp to −24 bp from 5′ UTR end+FKRP ORF+3′ UTR+MSEC
+1 bp to −23 bp from 5′ UTR end+FKRP ORF+3′ UTR+MSEC
+1 bp to −22 bp from 5′ UTR end+FKRP ORF+3′ UTR+MSEC
+1 bp to −21 bp from 5′ UTR end+FKRP ORF+3′ UTR+MSEC
+1 bp to −20 bp from 5′ UTR end+FKRP ORF+3′ UTR+MSEC
+1 bp to −19 bp from 5′ UTR end+FKRP ORF+3′ UTR+MSEC
+1 bp to −18 bp from 5′ UTR end+FKRP ORF+3′ UTR+MSEC
+1 bp to −17 bp from 5′ UTR end+FKRP ORF+3′ UTR+MSEC
+1 bp to −16 bp from 5′ UTR end+FKRP ORF+3′ UTR+MSEC
+1 bp to −15 bp from 5′ UTR end+FKRP ORF+3′ UTR+MSEC
+1 bp to −14 bp from 5′ UTR end+FKRP ORF+3′ UTR+MSEC
+1 bp to −13 bp from 5′ UTR end+FKRP ORF+3′ UTR+MSEC
+1 bp to −12 bp from 5′ UTR end+FKRP ORF+3′ UTR+MSEC
+1 bp to −11 bp from 5′ UTR end+FKRP ORF+3′ UTR+MSEC
+1 bp to −10 bp from 5′ UTR end+FKRP ORF+3′ UTR+MSEC
+1 bp to −9 bp from 5′ UTR end+FKRP ORF+3′ UTR+MSEC
+1 bp to −8 bp from 5′ UTR end+FKRP ORF+3′ UTR+MSEC
+1 bp to −7 bp from 5′ UTR end+FKRP ORF+3′ UTR+MSEC
+1 bp to −6 bp from 5′ UTR end+FKRP ORF+3′ UTR+MSEC
+1 bp to −5 bp from 5′ UTR end+FKRP ORF+3′ UTR+MSEC
+1 bp to −4 bp from 5′ UTR end+FKRP ORF+3′ UTR+MSEC
+1 bp to −3 bp from 5′ UTR end+FKRP ORF+3′ UTR+MSEC
+1 bp to −2 bp from 5′ UTR end+FKRP ORF+3′ UTR+MSEC
+1 bp to −1 bp from 5′ UTR end+FKRP ORF+3′ UTR+MSEC FKRP ORF+3′ UTR+MSEC

### G-Quadruplex

+1 bp to −39 bp from 5′ UTR end+FKRP ORF+3′ UTR+MSEC
−40 bp to −143 bp from 5′ UTR end+FKRP ORF+3′ UTR+MSEC

### Secondary Structures

−39 bp to −56 bp from 5′ UTR end+FKRP ORF+3′ UTR+MSEC
+1 bp to −38 bp and −57 bp to −143 bp from 5′ UTR end+FKRP ORF+3′ UTR+MSEC

### Hairpin

−56 bp to −102 bp from 5′ UTR end+FKRP ORF+3′ UTR+MSEC
+1 bp to −55 bp and −103 bp to −143 bp from 5′ UTR end+FKRP ORF+3′ UTR+MSEC

### Secondary Structures

−102 bp to −123 bp from 5′ UTR end+FKRP ORF+3′ UTR+MSEC
+1 bp to −101 bp and −124 bp to −143 bp from 5′ UTR end+FKRP ORF+3′ UTR+MSEC
   3′ UTR truncations
5′ UTR+FKRP ORF+1 bp to +1600 bp at 3′ UTR end+MSEC
5′ UTR+FKRP ORF+1 bp to +1500 bp at 3′ UTR end+MSEC
5′ UTR+FKRP ORF+1 bp to +1400 bp at 3′ UTR end+MSEC
5′ UTR+FKRP ORF+1 bp to +1300 bp at 3′ UTR end+MSEC
5′ UTR+FKRP ORF+1 bp to +1200 bp at 3′ UTR end+MSEC
5′ UTR+FKRP ORF+1 bp to +1100 bp at 3′ UTR end+MSEC
5′ UTR+FKRP ORF+1 bp to +1000 bp at 3′ UTR end+MSEC
5′ UTR+FKRP ORF+1 bp to +900 bp at 3′ UTR end+MSEC
5′ UTR+FKRP ORF+1 bp to +800 bp at 3′ UTR end+MSEC
5′ UTR+FKRP ORF+1 bp to +700 bp at 3′ UTR end+MSEC
5′ UTR+FKRP ORF+1 bp to +600 bp at 3′ UTR end+MSEC
5′ UTR+FKRP ORF+1 bp to +500 bp at 3′ UTR end+MSEC
5′ UTR+FKRP ORF+1 bp to +400 bp at 3′ UTR end+MSEC
5′ UTR+FKRP ORF+1 bp to +300 bp at 3′ UTR end+MSEC
5′ UTR+FKRP ORF+1 bp to +200 bp at 3′ UTR end+MSEC
5′ UTR+FKRP ORF+1 bp to +100 bp at 3′ UTR end+MSEC
5′ UTR+FKRP ORF+1 bp to +01 bp at 3′ UTR end+MSEC
5′ UTR+FKRP ORF+MSEC
5′ UTR+FKRP ORF+01 bp to +1600 bp at 3′ UTR end+MSEC
5′ UTR+FKRP ORF+100 bp to +1600 bp at 3′ UTR end+MSEC
5′ UTR+FKRP ORF+200 bp to +1600 bp at 3′ UTR end+MSEC
5′ UTR+FKRP ORF+300 bp to +1600 bp at 3′ UTR end+MSEC
5′ UTR+FKRP ORF+400 bp to +1600 bp at 3′ UTR end+MSEC
5′ UTR+FKRP ORF+500 bp to +1600 bp at 3′ UTR end+MSEC
5′ UTR+FKRP ORF+600 bp to +1600 bp at 3′ UTR end+MSEC
5′ UTR+FKRP ORF+700 bp to +1600 bp at 3′ UTR end+MSEC
5′ UTR+FKRP ORF+800 bp to +1600 bp at 3′ UTR end+MSEC
5′ UTR+FKRP ORF+900 bp to +1600 bp at 3′ UTR end+MSEC
5′ UTR+FKRP ORF+1000 bp to +1600 bp at 3′ UTR end+MSEC
5′ UTR+FKRP ORF+1100 bp to +1600 bp at 3′ UTR end+MSEC
5′ UTR+FKRP ORF+1200 bp to +1600 bp at 3′ UTR end+MSEC
5′ UTR+FKRP ORF+1300 bp to +1600 bp at 3′ UTR end+MSEC
5′ UTR+FKRP ORF+1400 bp to +1600 bp at 3′ UTR end+MSEC
5′ UTR+FKRP ORF+1500 bp to +1600 bp at 3′ UTR end+MSEC
5′ UTR+FKRP ORF+1600 bp to +1600 bp at 3′ UTR end+MSEC
FKRP ORF+MSEC

### REFERENCES

1 Nickolls A R, Bonnemann C G. The roles of dystroglycan in the nervous system: insights from animal models of muscular dystrophy. Dis Model Mech. Dec. 19 2018; 11 (12) doi: 10.1242/dmm.035931
2. Ervasti J M, Campbell K P. A role for the dystrophin-glycoprotein complex as a transmembrane linker between laminin and actin. J Cell Biol. August 1993; 122 (4): 809-23. doi: 10.1083/jcb. 122.4.809
3. Praissman J L, Willer T, Sheikh M O, et al. The functional O-mannose glycan on alpha-dystroglycan contains a phospho-ribitol primed for matriglycan addition. Elife. Apr. 29 2016; 5doi: 10.7554/eLife.14473
4. Mercuri E, Brockington M, Straub V, et al. Phenotypic spectrum associated with mutations in the fukutin-related protein gene. Ann Neurol. April 2003; 53 (4): 537-42. doi: 10.1002/ana.10559
5. Boito C A, Melacini P, Vianello A, et al. Clinical and molecular characterization of patients with limb-girdle muscular dystrophy type 21. Arch Neurol. December 2005; 62 (12): 1894-9. doi: 10.1001/archneur.62.12.1894
6. Sveen M L, Schwartz M, Vissing J. High prevalence and phenotype-genotype correlations of limb girdle muscular dystrophy type 21 in Denmark. Ann Neurol. May 2006; 59 (5): 808-15. doi: 10.1002/ana.20824
7. Keramaris-Vrantsis E, Lu P J, Doran T, et al. Fukutin-related protein localizes to the Golgi apparatus and mutations lead to mislocalization in muscle in vivo. Muscle Nerve. October 2007; 36 (4): 455-65. doi: 10.1002/mus.20833
8. Esapa C T, McIlhinney R A, Blake D J. Fukutin-related protein mutations that cause congenital muscular dystrophy result in E R-retention of the mutant protein in cultured cells. Hum Mol Genet. Jan. 15 2005; 14 (2): 295-305. doi: 10.1093/hmg/ddi026
9. Brockington M, Blake D J, Prandini P, et al. Mutations in the fukutin-related protein gene (FKRP) cause a form of congenital muscular dystrophy with secondary laminin alpha2 deficiency and abnormal glycosylation of alpha-dystroglycan. Am J Hum Genet. December 2001; 69 (6): 1198-209. doi: S0002-9297 (07) 61249-X [pii] 10.1086/324412
10. Tucker J D, Lu P J, Xiao X, Lu Q L. Overexpression of Mutant FKRP Restores Functional Glycosylation and Improves Dystrophic Phenotype in FKRP Mutant Mice. Mol Ther Nucleic Acids. Jun. 1 2018; 11:216-227. doi: 10.1016/j.omtn.2018.02.008
11. Xu L, Lu P J, Wang C H, et al. Adeno-associated virus 9 mediated FKRP gene therapy restores functional glycosylation of alpha-dystroglycan and improves muscle functions. Mol Ther. October 2013; 21 (10): 1832-40. doi: 10.1038/mt.2013.156
12. Vannoy C H, Xiao W, Lu P, Xiao X, Lu Q L. Efficacy of Gene Therapy Is Dependent on Disease Progression in Dystrophic Mice with Mutations in the FKRP Gene. Mol Ther Methods Clin Dev. Jun. 16 2017; 5:31-42. doi: 10.1016/j.omtm.2017.02.002
13. Gicquel E, Maizonnier N, Foltz S J, et al. AAV-mediated transfer of FKRP shows therapeutic efficacy in a murine model but requires control of gene expression. Hum Mol Genet. May 15 2017; 26 (10): 1952-1965. doi: 10.1093/hmg/ddx066
14. Chan Y M, Keramaris-Vrantsis E, Lidov H G, et al. Fukutin-related protein is essential for mouse muscle, brain and eye development and mutation recapitulates the wide clinical spectrums of dystroglycanopathies. Hum Mol Genet. Oct. 15 2010; 19 (20): 3995-4006. doi: 10.1093/hmg/ddq314
15. Blaeser A, Awano H, Wu B, Lu Q L. Progressive Dystrophic Pathology in Diaphragm and Impairment of Cardiac Function in FKRP P448L Mutant Mice. PLOS ONE. 2016; 11 (10): e0164187. doi: 10.1371/journal.pone.0164187
16. Qiao C, Wang C H, Zhao C, et al. Muscle and heart function restoration in a limb girdle muscular dystrophy 2I (LGMD2I) mouse model by systemic FKRP gene delivery. Mol Ther. November 2014; 22 (11): 1890-9. doi: 10.1038/mt.2014.141
17. Krag T O, Vissing J. A New Mouse Model of Limb-Girdle Muscular Dystrophy Type 21 Homozygous for the Common L276I Mutation Mimicking the Mild Phenotype in Humans. J Neuropathol Exp Neurol. December 2015; 74 (12): 1137-46. doi: 10.1097/NEN.0000000000000260
18. Vannoy C H, Leroy V, Lu Q L. Dose-Dependent Effects of FKRP Gene-Replacement Therapy on Functional Rescue and Longevity in Dystrophic Mice. Mol Ther Methods Clin Dev. Dec. 14 2018; 11:106-120. doi: 10.1016/j.omtm.2018.10.004
19. Kregel C K, Allen D L, Booth F W, et al. Resource book for the design of animal exercise protocols. American Physiological Society; 2006. http://the-aps.org/pa/action/exercise/book.pdf
20. De Luca A. Use of treadmill and wheel exercise for impact on mdx mice phenotype. TREAT-NMD Activity A07: Accelerate preclinical phase of new therapeutic treatment development. DMD_M.2.1.001. TREAT-NMD Neuromuscular Network; 2011 (2.0). http://treat-nmd.eu/resources/research-resources/dmd-sops/
21. Grange R W. Use of treadmill and wheel exercise to assess dystrophic state. TREAT-NM Activity A07: Accelerate preclinical phase of new therapeutic treatment development. DMD_M.2.1.003. TREAT-NMD Neuromuscular Network; 2011 (1.0). http://treat-nmd.eu/resources/research-resources/dmd-sops/
22. Bye P T, Esau S A, Walley K R, Macklem P T, Pardy R L. Ventilatory muscles during exercise in air and oxygen in normal men. J Appl Physiol. February 1984; 56 (2): 464-71.
23. Mahler D A, Moritz E D, Loke J. Ventilatory responses at rest and during exercise in marathon runners. J Appl Physiol. February 1982; 52 (2): 388-92.
24. Bassett D R, Jr., Howley E T. Limiting factors for maximum oxygen uptake and determinants of endurance performance. Med Sci Sports Exerc. January 2000; 32 (1): 70-84.
25. Lindstedt S L, Thomas R G, Leith D E. Does peak inspiratory flow contribute to setting VO2max? A test of symmorphosis. Respiration physiology. January 1994; 95 (1): 109-18.
26. Spurway N C, Ekblom B, Noakes T D, Wagner P D. What limits VO₂max? A symposium held at the BASES Conference, 6 Sep. 2010. Journal of sports sciences. 2012; 30 (6): 517-31. doi: 10.1080/02640414.2011.642809
27. De Luca A, Pierno S, Liantonio A, et al. Enhanced dystrophic progression in mdx mice by exercise and beneficial effects of taurine and insulin-like growth factor-1. J Pharmacol Exp Ther. January 2003; 304 (1): 453-63.
28. Okano T, Yoshida K, Nakamura A, et al. Chronic exercise accelerates the degeneration-regeneration cycle and downregulates insulin-like growth factor-1 in muscle of mdx mice. Muscle Nerve. August 2005; 32 (2): 191-9. doi: 10.1002/mus.20351
29. Rocco A B, Levalley J C, Eldridge J A, Marsh S A, Rodgers B D. A novel protocol for assessing exercise performance and dystropathophysiology in the mdx mouse. Muscle Nerve. October 2014; 50 (4): 541-8. doi: 10.1002/mus.24184
30. Maricelli J W, Kagel D R, Bishaw Y M, Nelson O L, Lin D C, Rodgers B D. Sexually Dimorphic Skeletal Muscle and Cardiac Dysfunction in a Mouse Model of Limb Girdle Muscular Dystrophy 2i. J Appl Physiol (1985). Jun. 29 2017: jap 00287 2017. doi: 10.1152/japplphysiol.00287.2017
31. High-dose AAV gene therapy deaths. Nat Biotechnol. August 2020; 38 (8): 910. doi: 10.1038/s41587-020-0642-9
32. Wilson J M, Flotte T R. Moving Forward After Two Deaths in a Gene Therapy Trial of Myotubular Myopathy. Hum Gene Ther. July 2020; 31 (13-14): 695-696. doi: 10.1089/hum.2020.182
33. Weinmann J, Weis S, Sippel J, et al. Identification of a myotropic AAV by massively parallel in vivo evaluation of barcoded capsid variants. Nat Commun. Oct. 28 2020; 11 (1): 5432. doi: 10.1038/s41467-020-19230-w
34. El Andari J, Renaud-Gabardos E, Tulalamba W, et al. Semirational bioengineering of AAV vectors with increased potency and specificity for systemic gene therapy of muscle disorders. Sci Adv. Sep. 23 2022; 8 (38): eabn4704. doi: 10.1126/sciadv.abn4704
35. Tabebordbar M, Lagerborg K A, Stanton A, et al. Directed evolution of a family of AAV capsid variants enabling potent muscle-directed gene delivery across species. Cell. Sep. 16 2021; 184 (19): 4919-4938 e22. doi: 10.1016/j.cell.2021.08.028
36. Lukowski S W, Bombieri C, Trezise A E. Disrupted post-transcriptional regulation of the cystic fibrosis transmembrane conductance regulator (CFTR) by a 5′UTR mutation is associated with a CFTR-related disease. Hum Mutat. October 2011; 32 (10): E2266-82. doi: 10.1002/humu.21545
37. Lukowski S W, Rothnagel J A, Trezise A E. CFTR mRNA expression is regulated by an upstream open reading frame and RNA secondary structure in its 5′ untranslated region. Hum Mol Genet. Feb. 15 2015; 24 (4): 899-912. doi: 10.1093/hmg/ddu501
38. Leppek K, Das R, Barna M. Functional 5′ UTR mRNA structures in eukaryotic translation regulation and how to find them. Nat Rev Mol Cell Biol. March 2018; 19 (3): 158-174. doi: 10.1038/nrm.2017.103
39. Cammas A, Millevoi S. RNA G-quadruplexes: emerging mechanisms in disease. Nucleic Acids Res. Feb. 28 2017; 45 (4): 1584-1595. doi: 10.1093/nar/gkw1280
40. Fay M M, Lyons S M, Ivanov P. RNA G-Quadruplexes in Biology: Principles and Molecular Mechanisms. J Mol Biol. Jul. 7 2017; 429 (14): 2127-2147. doi: 10.1016/j.jmb.2017.05.017
41. Salva M Z, Himeda C L, Tai P W, et al. Design of tissue-specific regulatory cassettes for high-level rAAV-mediated expression in skeletal and cardiac muscle. Mol Ther. February 2007; 15 (2): 320-9.
42. Himeda C L, Chen X, Hauschka S D. Design and testing of regulatory cassettes for optimal activity in skeletal and cardiac muscles. Methods Mol Biol. 2011; 709:3-19. doi: 10.1007/978-1-61737-982-6_1
43. Goncalves M A, Janssen J M, Nguyen Q G, et al. Transcription factor rational design improves directed differentiation of human mesenchymal stem cells into skeletal myocytes. Mol Ther. July 2011; 19 (7): 1331-41. doi: 10.1038/mt.2010.308
44. Martari M, Sagazio A, Mohamadi A, et al. Partial rescue of growth failure in growth hormone (GH)-deficient mice by a single injection of a double-stranded adeno-associated viral vector expressing the GH gene driven by a muscle-specific regulatory cassette. Hum Gene Ther. July 2009; 20 (7): 759-66. doi: 10.1089/hum.2008.197
45. Bengtsson N E, Seto J T, Hall J K, Chamberlain J S, Odom G L. Progress and prospects of gene therapy clinical trials for the muscular dystrophies. Hum Mol Genet. Apr. 15 2016; 25 (R1): R9-17. doi: 10.1093/hmg/ddv420
46. Hu C, Kasten J, Park H, et al. Myocyte-mediated arginase expression controls hyperargininemia but not hyperammonemia in arginase-deficient mice. Mol Ther. October 2014; 22 (10): 1792-802. doi: 10.1038/mt.2014.99
47. Muir L A, Nguyen Q G, Hauschka S D, Chamberlain J S. Engraftment potential of dermal fibroblasts following in vivo myogenic conversion in immunocompetent dystrophic skeletal muscle. Mol Ther Methods Clin Dev. Jun. 25 2014; 1:14025. doi: 10.1038/mtm.2014.25
48. Blankinship M J, Gregorevic P, Allen J M, et al. Efficient transduction of skeletal muscle using vectors based on adeno-associated virus serotype 6. Mol Ther. October 2004; 10 (4): 671-8. doi: 10.1016/j.ymthe.2004.07.016
49. Gao G, Vandenberghe L H, Alvira M R, et al. Clades of Adeno-associated viruses are widely disseminated in human tissues. J Virol. June 2004; 78 (12): 6381-8.
50. Alhamidi M, Kjeldsen Buvang E, Fagerheim T, et al. Fukutin-related protein resides in the Golgi cisternae of skeletal muscle fibres and forms disulfide-linked homodimers via an N-terminal interaction. PLOS One. 2011; 6 (8): e22968. doi: 10.1371/journal.pone.0022968
51. Gregorevic P, Blankinship M J, Allen J M, et al. Systemic delivery of genes to striated muscles using adeno-associated viral vectors. Nat Med. August 2004; 10 (8): 828-34.
52. Yin F C, Spurgeon H A, Rakusan K, Weisfeldt M L, Lakatta E G. Use of tibial length to quantify cardiac hypertrophy: application in the aging rat. Am J Physiol. December 1982; 243 (6): H941-7. doi: 10.1152/ajpheart. 1982.243.6.H941
53. Kishimoto T K, Samulski R J. Addressing high dose AAV toxicity-‘one and done’ or ‘slower and lower’? Expert Opin Biol Ther. September 2022; 22 (9): 1067-1071. doi: 10.1080/14712598.2022.2060737
54. Ertl H C J. Immunogenicity and toxicity of AAV gene therapy. Front Immunol. 2022; 13:975803. doi: 10.3389/fimmu.2022.975803
55. Mendell J R, Al-Zaidy S A, Rodino-Klapac L R, et al. Current Clinical Applications of In Vivo Gene Therapy with AAVs. Mol Ther. Feb. 3 2021; 29 (2): 464-488. doi: 10.1016/j.ymthe.2020.12.007
56. Bonnemann C G, Belluscio B A, Braun S, Morris C, Muntoni F. A collaborative analysis by clinical trial sponsors and academic experts of anti-transgene SAEs in studies of gene therapy for DMD. Mol Ther. 2022; 30 (5S1): 4.
57. Cheng A, Liu C, Ye W, et al. Selective C9orf72 G-Quadruplex-Binding Small Molecules Ameliorate Pathological Signatures of ALS/FTD Models. J Med Chem. Oct. 13 2022; 65 (19): 12825-12837. doi: 10.1021/acs.jmedchem.2c00654
58. Hordeaux J, Buza E L, Jeffrey B, et al. MicroRNA-mediated inhibition of transgene expression reduces dorsal root ganglion toxicity by AAV vectors in primates. Sci Transl Med. Nov. 11 2020; 12 (569) doi: 10.1126/scitranslmed.aba9188
59. Cao J, Novoa E M, Zhang Z, et al. High-throughput 5′ UTR engineering for enhanced protein production in non-viral gene therapies. Nat Commun. Jul. 6 2021; 12 (1): 4138. doi: 10.1038/s41467-021-24436-7
60. Wasala N B, Yue Y, Vance J, Duan D. Uniform low-level dystrophin expression in the heart partially preserved cardiac function in an aged mouse model of Duchenne cardiomyopathy. J Mol Cell Cardiol. January 2017; 102:45-52. doi: S0022-2828 (16) 30425-4 [pii] 10.1016/j.yjmcc.2016.11.011
61. Keramaris E, Lu P J, Tucker J, Lu Q L. Expression of glycosylated alpha-dystroglycan in newborn skeletal and cardiac muscles of fukutin related protein (FKRP) mutant mice. Muscle Nerve. April 2017; 55 (4): 582-590. doi: 10.1002/mus.25378
62. Kregel K C, Allen D L, Booth F W, Fleshner M R, Henrikson E J, Musch T I. Resource Book for the Design of Animal Exercise Protocols. 2006.
63. Maricelli J W, Kagel D R, Bishaw Y M, Nelson O L, Lin D C, Rodgers B D. Sexually dimorphic skeletal muscle and cardiac dysfunction in a mouse model of limb girdle muscular dystrophy 2i. J Appl Physiol (1985). Nov. 1 2017; 123 (5): 1126-1138. doi: 10.1152/japplphysiol.00287.2017
64. Konopka A R, Harber M P. Skeletal muscle hypertrophy after aerobic exercise training. Exerc Sport Sci Rev. April 2014; 42 (2): 53-61. doi: 10.1249/JES.0000000000000007
65. Gehrig S M, Koopman R, Naim T, Tjoakarfa C, Lynch G S. Making fast-twitch dystrophic muscles bigger protects them from contraction injury and attenuates the dystrophic pathology. Am J Pathol. January 2010; 176 (1): 29-33. doi: 10.2353/ajpath.2010.090760
66. Webster C, Silberstein L, Hays A P, Blau H M. Fast muscle fibers are preferentially affected in Duchenne muscular dystrophy. Cell. Feb. 26 1988; 52 (4): 503-13.
67. Halbert C L, Allen J M, Chamberlain J S. AAV6 Vector Production and Purification for Muscle Gene Therapy. Methods Mol Biol. 2018; 1687:257-266. doi: 10.1007/978-1-4939-7374-3 18
68. Speakman J R. Measuring energy metabolism in the mouse-theoretical, practical, and analytical considerations. Front Physiol. 2013; 4:34. doi: 10.3389/fphys.2013.00034
69. Maricelli J, Bishaw Y, Wang B, Du M, Rodgers B D. Systemic Smad7 Gene Therapy Increases Striated Muscle Mass and Enhances Exercise Capacity in a Dose-Dependent Manner. Hum Gene Ther. Oct. 1 2017; doi: 10.1089/hum.2017.158
70. Banks G B, Chamberlain J S. The value of mammalian models for duchenne muscular dystrophy in developing therapeutic strategies. Curr Top Dev Biol. 2008; 84:431-53. doi: 10.1016/S0070-2153 (08) 00609-1
   SEQ ID NO. 1 is a human FKRP cDNA including 5′ and 3′ UTRs:

attgctccaagatggcggcggcggcggcagcgggagcgcagctcagctgg
gctggaactgccctcctggaactcccccagcctacaacctaggaggtgca
gggactgaggctcaggccaaatcgcaactcagacccagtgaacccaaggc
ctgaagagaatttggattcatttaccttgttttgtggggactggagagac
aagtaaactctcagagtaactgtcccctctgactaccatttctaaggcaa
gccccctgtttctactcttgcgccccctgctggtttcctgccctgtctga
tgccccggaggcccagctagccccagacttcggccccATGCGGCTCACCC
GCTGCCAGGCTGCCCTGGCGGCCGCCATCACCCTCAACCTTCTGGTCCTC
TTCTATGTCTCGTGGCTGCAGCACCAGCCTAGGAATTCCCGGGCCCGGGG
GCCCCGTCGTGCCTCTGCTGCCGGCCCCCGTGTCACCGTCCTGGTGCGGG
AGTTCGAGGCATTTGACAACGCGGTGCCCGAGCTGGTAGACTCCTTCCTG
CAGCAAGACCCAGCCCAGCCCGTGGTGGTGGCAGCCGACACGCTCCCCTA
CCCGCCCCTGGCCCTGCCCCGCATCCCCAACGTGCGTCTGGCGCTGCTCC
AGCCCGCCCTGGACCGGCCAGCCGCAGCCTCGCGCCCGGAGACCTACGTG
GCCACCGAGTTTGTGGCCCTAGTACCTGATGGGGCGCGGGCTGAGGCACC
TGGCCTGCTGGAGCGCATGGTGGAGGCGCTCCGCGCAGGAAGCGCACGTC
TGGTGGCCGCCCCGGTTGCCACGGCCAACCCTGCCAGGTGCCTGGCCCTG
AACGTCAGCCTGCGAGAGTGGACCGCCCGCTATGGCGCAGCCCCCGCCGC
GCCCCGCTGCGACGCCCTGGACGGAGATGCTGTGGTGCTCCTGCGCGCCC
GCGACCTCTTCAACCTCTCGGCGCCCCTGGCCCGGCCGGTGGGCACCAGC
CTCTTTCTGCAGACCGCCCTTCGCGGCTGGGCGGTGCAGCTGCTGGACTT
GACCTTCGCCGCGGCGCGCCAGCCCCCGCTGGCCACGGCCCACGCGCGCT
GGAAGGCTGAGCGCGAGGGACGCGCTCGGCGGGCGGCGCTGCTCCGCGCG
CTGGGCATCCGCCTAGTGAGCTGGGAAGGCGGGCGGCTGGAGTGGTTCGG
CTGCAACAAGGAGACCACGCGCTGCTTCGGAACCGTGGTGGGCGACACGC
CCGCCTACCTCTACGAGGAGCGCTGGACGCCCCCCTGCTGCCTGCGCGCG
CTGCGCGAGACCGCCCGCTATGTGGTGGGCGTGCTGGAGGCTGCGGGCGT
GCGCTACTGGCTCGAGGGCGGCTCACTGCTGGGGGCCGCCCGCCACGGGG
ACATCATCCCATGGGACTACGACGTGGACCTGGGCATCTACTTGGAGGAC
GTGGGCAACTGCGAGCAGCTGCGGGGGGCAGAGGCCGGCTCGGTGGTGGA
TGAGCGCGGCTTCGTATGGGAGAAGGCGGTCGAGGGCGACTTTTTCCGCG
TGCAGTACAGCGAAAGCAACCACTTGCACGTGGACCTGTGGCCCTTCTAC
CCCCGCAATGGCGTCATGACCAAGGACACGTGGCTGGACCACCGGCAGGA
TGTGGAGTTTCCCGAGCACTTCCTGCAGCCGCTGGTGCCCCTGCCCTTTG
CCGGCTTCGTGGCGCAGGCGCCTAACAACTACCGCCGCTTCCTGGAGCTC
AAGTTCGGGCCCGGGGTCATCGAGAACCCCCAGTACCCCAACCCGGCACT
GCTGAGTCTGACGGGAAGCGGCTGAagccctgataacctcgcctttgttt
ttcgggggtctgtctggatgtggagaagctctgtgtgagcggtgaggggt
ggagggatgtcgcggagaggggaagggggaaactgaccaagaaagaaatt
ctaaggagagcatgagagaaggctggcattggcaggaggagagcaccagg
acgaggatgggaagcgacctccagatttatcaaatggtcatgcccactgg
gagccgtggatatgcgtggggacatcctgggtcatctcagtcatggaggg
agacggggatgtcacgccgtcccgcagggcccagcacagccccagacccg
aaaaaagtgttctgcccaagattccgagagccctgcgctctagggcaggg
gcagagttttggaaacagtgcaggctctggagccagactggcgagattca
aatcctggctctatcgcttcggagccaggtgggcctggggggggtcgcag
tctctctgtgcctcagttgcttccaggatgcgggacccttggctgcaggg
gttgcttccgccactagagggcgcgccggtcccgctcctggtggcccact
gtggctgcccgggcgacagtacgcccagggcctgtgttccatagccatct
actctcttgagcctttggacttctctccaagcccctgtgggaggcggaca
gcagtgaccacctccccttcttttggactgcgacctccttccctcctggg
agagccctgtgacctgcatgctactcttaactgttctattcaagactgaa
tagaagtatttcagtcttgcagaggaggaaatgctcagagctccgaggtg
cggctgtggtcgagaaccgggtgctgggccgggcgcgggggctcacgcct
gtaatcccagcactttgggaggccgaggtgggaggatcgcttgagcccag
gagtctgagaccagcctcggcaacatgccaagaccccgtctctattttta
aaaaagaaaaagaaccgacttctgaatcgcagctccactcatgactaata
cctcattatttcagctgtctgcacctaattccccacttgcacggcagtgt
agacaataaccatagctcacactcactgagcacctactgggtaccaggca
ccattctcagtgtttcacctggatcaactaatgcgtccctcacctcagcc
ctctgaagtgacagctgctattattttcattacacagatgaaaaagctga
ggccagaatcgtgaagtcacttgctcaaggtcaggcagcttaggaagggg
cagatcgggggcttgaacccaggtggtcaggctctggagcccacaattgt
cttacccactatgcccctctctagtcatggtccccaagaggggcttggag
acccacttagcaggtgaaagcaatggcagccttccttatttgattatgca
cctaagaataaatggtatttgggcatgtattcccaatatgtgtatattta
tttataaatatatacagatactattatctgtatgttagtaataaagctta
aattattccattttaaaattatgaatatgaatagggttttttttatgttt
cttgcctcatcccaatgacttttgcacacccaggtgtgagcacccagcat
tcaagaccacg

SEQ ID NO. 2 is a murine FKRP cDNA including 5′ and 3′ UTRs:

ATTGCTCCAAGATGGCGGCGGCGGCGGCGGCGGCAGGGCAGATGACCCAA
GGTGAAACCACTCTTCTTGATCACCCTCAGCCTGAAACCTAGGGAGGATG
CCCTGGACGCCCCAGCTAGGGTCTGACATCAGGCCCCATGCGGCTCACCC
GCTGCTGGGCTGCCCTGGCAGCCGCCATCATCCTCAACCTCCTAGTCTTC
TTCTATGTGTCATGGCTACAACACCAGCCCAGAAACTCCCGGGCCCGGGG
TCCCCGCCGGACTTCTGCCATTGGCCCCCGAGTCACCGTCCTGATTCGGG
AATTTGAGGCTTTTGACAACGCGGTGCCAGAGCTAGTGGATTCCTTTTTG
CAGCAGGACCCAGCCCAGCCCGTGGTAGTGGCGGCCGACACACTCCCTTA
CCCACCCCTGGCCTTGCCTCGCATCCCCAACGTTCGCCTGGCTCTGCTCC
AGCCAGCCCTGGACCGGCCAGCGGCGGCCTCGCGCCCGGAGACCTACGTA
GCCACCGAGTTTGTGGCTCTAGTGCCTGATGGAGCGCGGGCCGAGTCACC
AGGCCACCTGGAGCGAATGGTGGAGGCGCTCCGAGGGAGCAGCGCGCGCC
TAGTGGCCGCCCCGGTCGCCACCGCCAACCCAGCGCGGTGCCTAGCTCTG
AACGTCAGCCTGCGGGAGTGGACTGCGCGCTACGACCCAGCTCCCAGCGC
GCCCCGCTGCGACGCTTTGGATGGCGACGCTGTGCTGCTGATGCGCTCCC
GCGACCTCTTCAACCTCTCGGTGCCCCTGGCGCGGCCGCTGGCCACCAGC
CTCTTCCTACAGACCGCCCTGCGCGGCTGGGCAGTGCAGCTGCTGGACTT
GACCTTCGCCGCGGCGCGCCAGCCACCGCTGGCCACCGCCCACGCGCGCT
GGAAGGCGGAACGTGAGGGGCGCTCACGGAGAGCGGCGCTGCTGCGCTCG
TTGGGAATCCGTCTGGTGAGCTGGGAAGGCGGGCGGCTAGAGTGGTTTGG
CTGCAGCAAGGAGAGCGCGCGCTGCTTCGGTACGGTGGCGGGCGACACAC
CCGCCTACCTGTATGAGGGCCGCTGGACCCCACCTTGTTGCCTGCGCGCG
CTGCGCGAGACTGCGCGCTACGTGGTGGGCGTGCTGGAGGCGGCGGGCGT
GCGCTACTGGTTGGAGGGCGGCTCGCTGCTGGGTGCAGCTCGCCACGGCG
ACATCATCCCTTGGGACTACGACGTAGATCTGGGCATCTACCTGGAGGAC
GTGGGCAACTGCGAGCAGTTGCGGGGTGCCGAAGCTGGCTCGGTAGTGGA
TGAACGCGGCTTTGTGTGGGAGAAGGCGGTGGAGGGCGACTTCTTCCGAG
TACAGTACAGTGAGAACAACCACCTGCACGTGGACCTGTGGCCCTTTTAC
CCCCGCAATGGGGTTATGACCAAGGACACGTGGCTGGACCACCGGCAGGA
TGTTGAGTTCCCAGAGCACTTCCTGCAGCCACTTGTCCCCCTGCCCTTTG
CGGGTTTCATGGCACAGGCCCCTAACAACTACCGCCGCTTCCTGGAGCTG
AAGTTTGGGCCTGGGGTCATCGAGAACCCGGAGTACCCCAACCCCGCACT
CTTAAGCTTGACAGGCGGTTGAAGCCCTGGTACCCACATCTGGGGCTAGG
TGAACAGCAGGGTACAAGAGTCTGTTCTTGCCTCGGATTGTGTATGGATC
TGAACCAGTTTGTGATTGGGTAGTGGGGCTGTGCACTGGAGGGAAGAAAA
AAAAAGGGTGAACTGGCCAAGGAAGATTGGAGGGAACACACAGGACAGTT
TCAGCTTTGGCGGGAAGCAGAACCCAAAGACTGGAGCAACTCAGCCTCAT
GAGGGAGGAAAGTGGTGGCCTTTGATAGAAAAGCCCAGGGAGTCTTTAGG
GCTCTGCATAGTCTCTGCCCTATATTCCAAGAGCTGTAGGCCCTTGGAGA
GTGAGATCCGGAGAACAGACGAGCCAGCCTCAGTCTACCACATACAACTT
AGGTATCCCTGGACCAGTTAGTTACCCAGTCTGTTTCATTTGAGATACCG
AATGCCTCCCTTTGTGTTTAGGATCCTCAGGACGCTTGGCTCCAGTGCTT
GCTTCCTCCACTAGAGGGCACTTCTGTCCCGCTTCAGTTCGTGTTTGATG
ACCGCTCGCGGCTCTGCTTCATCACCTTCATCTCTGGTTTTCTTGGAGTG
GCTCTGGGAGGTGGGACAGCACTGGCCACCTCCTCTCCTCCCCGAATGTG
GCACTTTCCTTTGAGAGTGCTGCTTGATGTATCTGCTGCTTCTAACCCCA
TCTTGCTCTTGGAGGACTGACTGGGCTCTCTCTGGTTTGGGATCTGGCCA
ATGCATTACCTCTTTATTTCAGCCCTCTGCACCTGTTCCCCGCCTTCCCC
CTCCCTTCTAGGAAGGGATAAACCTTCGCCATAGCTCACATTTACAAAGA
GCTTTCGGGCTCAGGCATACACTCGGAAGTGACAGCTGCTATTACGTCCA
TTTTCCGGGGAGGAAAACTGAGGCCAAAATGACAGTCACTTGCCAGTAAT
CAGATATTTTAGGAAGTGGCTAGTAAGGGACTTGAATTCAGATCACCGTT
TCAAGGCCCACACTTCATTTCCTCTCTAGGTTCTGCTGTAGAAATAGTAC
CCAAGTGGAGCCTTGCCATGGCTGTGCCACTCAAACAGGTTTAAAAAAAA
ATTCTCATTTAATTATGCATATAAATTATGTTCAAACCAGTCTGTATATT
GTTAAATAAGACTTAAATTATGCTACTTTAAAGTTTAAAAATTCAAGACA
AATAGAAAAAAAAAAAA

SEQ ID NO. 3 is a murine FKRP 5′ untranslated sequence:

ATTGCTCCAAGATGGCGGCGGCGGCGGCGGCGGGGCAGATGACCCAAGGT
GAAACCACTCTTCTTGATCACCCTCAGCCTGAAACCTAGGGAGGATGCCC
TGGACGCCCCAGCTAGGGTCTGACATCAGGCCCC

SEQ ID NO. 4 is a murine FKRP 3′ untranslated sequence:

AGCCCTGGTACCCACATCTGGGGCTAGGTGAACAGCAGGGTACAAGAGTC
TGTTCTTGCCTCGGATTGTGTATGGATCTGAACCAGTTTGTGATTGGGTA
GTGGGGCTGTGCACTGGAGGGAAGAAAAAAAAAGGGTGAACTGGCCAAGG
AAGATTGGAGGGAACACACAGGACAGTTTCAGCTTTGGCGGGAAGCAGAA
CCCAAAGACTGGAGCAACTCAGCCTCATGAGGGAGGAAAGTGGTGGCCTT
TGATAGAAAAGCCCAGGGAGTCTTTAGGGCTCTGCATAGTCTCTGCCCTA
TATTCCAAGAGCTGTAGGCCCTTGGAGAGTGAGATCCGGAGAACAGACGA
GCCAGCCTCAGTCTACCACATACAACTTAGGTATCCCTGGACCAGTTAGT
TACCCAGTCTGTTTCATTTGAGATACCGAATGCCTCCCTTTGTGTTTAGG
ATCCTCAGGACGCTTGGCTCCAGTGCTTGCTTCCTCCACTAGAGGGCACT
TCTGTCCCGCTTCAGTTCGTGTTTGATGACCGCTCGCGGCTCTGCTTCAT
CACCTTCATCTCTGGTTTTCTTGGAGTGGCTCTGGGAGGTGGGACAGCAC
TGGCCACCTCCTCTCCTCCCCGAATGTGGCACTTTCCTTTGAGAGTGCTG
CTTGATGTATCTGCTGCTTCTAACCCCATCTTGCTCTTGGAGGACTGACT
GGGCTCTCTCTGGTTTGGGATCTGGCCAATGCATTACCTCTTTATTTCAG
CCCTCTGCACCTGTTCCCCGCCTTCCCCCTCCCTTCTAGGAAGGGATAAA
CCTTCGCCATAGCTCACATTTACAAAGAGCTTTCGGGCTCAGGCATACAC
TCGGAAGTGACAGCTGCTATTACGTCCATTTTCCGGGGAGGAAAACTGAG
GCCAAAATGACAGTCACTTGCCAGTAATCAGATATTTTAGGAAGTGGCTA
GTAAGGGACTTGAATTCAGATCACCGTTTCAAGGCCCACACTTCATTTCC
TCTCTAGGTTCTGCTGTAGAAATAGTACCCAAGTGGAGCCTTGCCATGGC
TGTGCCACTCAAACAGGTTTAAAAAAAAATTCTCATTTAATTATGCATAT
AAATTATGTTCAAACCAGTCTGTATATTGTTAAATAAGACTTAAATTATG
CTACTTTAAAGTTTAAAAATTCAAGACAAATAGAAAAAAAAAAAA

SEQ ID NO. 6 is a murine FKRP cDNA sequence containing a mutated Kozak sequence:

ATTGCTCCAAGATGGCGGCGGCGGCGGCAGCGGGAGCGCAGCTCAGCTGGG
CTGGAACTGCCCTCCTGGAACTCCCCCAGCCTACAACCTAGGAGGTGCAGG
GACTGAGGCTCAGGCCAAATCGCAACTCAGACCCAGTGAACCCAAGGCCTG
AAGAGAATTTGGATTCATTTACCTTGTTTTGTGGGGACTGGAGAGACAAGT
AAACTCTCAGAGTAACTGTCCCCTCTGACTACCATTTCTAAGGATGCCCCG
GAGGCCCAGCTAGCCCCAGACTTCTCCACCATGCGGCTCACCCGCTGCCAG
GCTGCCCTGGCGGCCGCCATCACCCTCAACCTTCTGGTCCTCTTCTATGTC
TCGTGGCT . . .

SEQ ID NO. 7 is a murine FKRP cDNA sequence lacking the 5′ UTR:

. . . AGACTCAGCACTTAGTTTAGGAACCAGTGAGCAAGTCAGCCCTTG
GGGCAGCCCATACAAGGCCATGGGGCTGGGCAAGCTGCACGCCTGGGTCCG
GGGTGGGCACGGTGCCCGGGCAACGAGCTGAAAGCTCATCTGCTCTCAGGG
GCCCCTCCCTGGGGACAGCCCCTCCTGGCTAGTCACACCCTGTAGGCTCCT
CTATATAACCCAGGGGCACAGGGGCTGCCCTCATTCTACCACCACCTCCAC
AGCACAGACAGACACTCAGGAGCCAGCCAGCGTCGAGATGCGGCTCACCCG
CTGCCAGGCTGCCCTGGCGGCCGCCATCACCCTCAACCTTCTGGTCCTCTT
CTATGTCTCGTGGCT . . .

SEQ ID NO. 8 is the CK8e promoter:

TGCCCATGTAAGGAGGCAAGGCCTGGGGACACCCGAGATGCCTGGTTATAA
TTAACCCAGACATGTGGCTGCCCCCCCCCCCCCAACACCTGCTGCCTCTAA
AAATAACCCTGCATGCCATGTTCCCGGCGAAGGGCCAGCTGTCCCCCGCCA
GCTAGACTCAGCACTTAGTTTAGGAACCAGTGAGCAAGTCAGCCCTTGGGG
CAGCCCATACAAGGCCATGGGGCTGGGCAAGCTGCACGCCTGGGTCCGGGG
TGGGCACGGTGCCCGGGCAACGAGCTGAAAGCTCATCTGCTCTCAGGGGCC
CCTCCCTGGGGACAGCCCCTCCTGGCTAGTCACACCCTGTAGGCTCCTCTA
TATAACCCAGGGGCACAGGGGCTGCCCTCATTCTACCACCACCTCCACAGC
ACAGACAGACACTCAGGAGCCAGCCAGC

SEQ ID NO. 9 is RNA G-quadruplex in exon 1 of human FKRP:

attgctccaagatggcggcggcggcggcagcg

SEQ ID NO 10 is RNA G-quadruplex in exon 1 of murine FKRP:

tgtacaattgctccaagatggcggcggcggcggcggcggcag

SEQ ID NO 11 is human FKRP mRNA transcript variant 2 mRNA, NM_001039885.3

CATTGCTCCAAGATGGCGGCGGCGGCGGCAGCGGGAGCGCAGCTCAGCTGGGCT
GGAACTGCCCTCCTGGAACTCCCCCAGCCTACAACCTAGGAGGTGCAGGGACTG
AGGCTCAGGCCAAATCGCAACTCAGACCCAGTGAACCCAAGGCCTGAAGAGAAT
TTGGATTCATTTACCTTGTTTTGTGGGGACTGGAGAGACAAGTAAACTCTCAGAG
TAACTGTCCCCTCTGACTACCATTTCTAAGGCAAGCCCCCTGTTTCTACTCTTGCG
CCCCCTGCTGGTTTCCTGCCCTGTCTGATGCCCCGGAGGCCCAGCTAGCCCCAGA
CTTCGGCCCCATGCGGCTCACCCGCTGCCAGGCTGCCCTGGCGGCCGCCATCACC
CTCAACCTTCTGGTCCTCTTCTATGTCTCGTGGCTGCAGCACCAGCCTAGGAATTC
CCGGGCCCGGGGGCCCCGTCGTGCCTCTGCTGCCGGCCCCCGTGTCACCGTCCTG
GTGCGGGAGTTCGAGGCATTTGACAACGCGGTGCCCGAGCTGGTAGACTCCTTCC
TGCAGCAAGACCCAGCCCAGCCCGTGGTGGTGGCAGCCGACACGCTCCCCTACC
CGCCCCTGGCCCTGCCCCGCATCCCCAACGTGCGTCTGGCGCTGCTCCAGCCCGC
CCTGGACCGGCCAGCCGCAGCCTCGCGCCCGGAGACCTACGTGGCCACCGAGTT
TGTGGCCCTAGTACCTGATGGGGCGCGGGCTGAGGCACCTGGCCTGCTGGAGCG
CATGGTGGAGGCGCTCCGCGCAGGAAGCGCACGTCTGGTGGCCGCCCCGGTTGC
CACGGCCAACCCTGCCAGGTGCCTGGCCCTGAACGTCAGCCTGCGAGAGTGGAC
CGCCCGCTATGGCGCAGCCCCCGCCGCGCCCCGCTGCGACGCCCTGGACGGAGA
TGCTGTGGTGCTCCTGCGCGCCCGCGACCTCTTCAACCTCTCGGCGCCCCTGGCC
CGGCCGGTGGGCACCAGCCTCTTTCTGCAGACCGCCCTTCGCGGCTGGGCGGTGC
AGCTGCTGGACTTGACCTTCGCCGCGGCGCGCCAGCCCCCGCTGGCCACGGCCCA
CGCGCGCTGGAAGGCTGAGCGCGAGGGACGCGCTCGGCGGGCGGCGCTGCTCCG
CGCGCTGGGCATCCGCCTAGTGAGCTGGGAAGGCGGGCGGCTGGAGTGGTTCGG
CTGCAACAAGGAGACCACGCGCTGCTTCGGAACCGTGGTGGGCGACACGCCCGC
CTACCTCTACGAGGAGCGCTGGACGCCCCCCTGCTGCCTGCGCGCGCTGCGCGAG
ACCGCCCGCTATGTGGTGGGCGTGCTGGAGGCTGCGGGCGTGCGCTACTGGCTC
GAGGGCGGCTCACTGCTGGGGGCCGCCCGCCACGGGGACATCATCCCATGGGAC
TACGACGTGGACCTGGGCATCTACTTGGAGGACGTGGGCAACTGCGAGCAGCTG
CGGGGGGCAGAGGCCGGCTCGGTGGTGGATGAGCGCGGCTTCGTATGGGAGAAG
GCGGTCGAGGGCGACTTTTTCCGCGTGCAGTACAGCGAAAGCAACCACTTGCAC
GTGGACCTGTGGCCCTTCTACCCCCGCAATGGCGTCATGACCAAGGACACGTGGC
TGGACCACCGGCAGGATGTGGAGTTTCCCGAGCACTTCCTGCAGCCGCTGGTGCC
CCTGCCCTTTGCCGGCTTCGTGGCGCAGGCGCCTAACAACTACCGCCGCTTCCTG
GAGCTCAAGTTCGGGCCCGGGGTCATCGAGAACCCCCAGTACCCCAACCCGGCA
CTGCTGAGTCTGACGGGAAGCGGCTGAAGCCCTGATAACCTCGCCTTTGTTTTTC
GGGGGTCTGTCTGGATGTGGAGAAGCTCTGTGTGAGCGGTGAGGGGTGGAGGGA
TGTCGCGGAGAGGGGAAGGGGGAAACTGACCAAGAAAGAAATTCTAAGGAGAG
CATGAGAGAAGGCTGGCATTGGCAGGAGGAGAGCACCAGGACGAGGATGGGAA
GCGACCTCCAGATTTATCAAATGGTCATGCCCACTGGGAGCCGTGGATATGCGTG
GGGACATCCTGGGTCATCTCAGTCATGGAGGGAGACGGGGATGTCACGCCGTCC
CGCAGGGCCCAGCACAGCCCCAGACCCGAAAAAAGTGTTCTGCCCAAGATTCCG
AGAGCCCTGCGCTCTAGGGCAGGGGCAGAGTTTTGGAAACAGTGCAGGCTCTGG
AGCCAGACTGGCGAGATTCAAATCCTGGCTCTATCGCTTCGGAGCCAGGTGGGC
CTGGGGGGGCGTCGCAGTCTCTCTGTGCCTCAGTTGCTTCCAGGATGCGGGACCC
TTGGCTGCAGGGGTTGCTTCCGCCACTAGAGGGCGCGCCGGTCCCGCTCCTGGTG
GCCCACTGTGGCTGCCCGGGCGACAGTACGCCCAGGGCCTGTGTTCCATAGCCAT
CTACTCTCTTGAGCCTTTGGACTTCTCTCCAAGCCCCTGTGGGAGGCGGACAGCA
GTGACCACCTCCCCTTCTTTTGGACTGCGACCTCCTTCCCTCCTGGGAGAGCCCTG
TGACCTGCATGCTACTCTTAACTGTTCTATTCAAGACTGAATAGAAGTATTTCAGT
CTTGCAGAGGAGGAAATGCTCAGAGCTCCGAGGTGCGGCTGTGGTCGAGAACCG
GGTGCTGGGCCGGGCGCGGGGGCTCACGCCTGTAATCCCAGCACTTTGGGAGGC
CGAGGTGGGAGGATCGCTTGAGCCCAGGAGTCTGAGACCAGCCTCGGCAACATG
CCAAGACCCCGTCTCTATTTTTAAAAAAGAAAAAGAACCGACTTCTGAATCGCA
GCTCCACTCATGACTAATACCTCATTATTTCAGCTGTCTGCACCTAATTCCCCACT
TGCACGGCAGTGTAGACAATAACCATAGCTCACACTCACTGAGCACCTACTGGG
TACCAGGCACCATTCTCAGTGTTTCACCTGGATCAACTAATGCGTCCCTCACCTC
AGCCCTCTGAAGTGACAGCTGCTATTATTTTCATTACACAGATGAAAAAGCTGAG
GCCAGAATCGTGAAGTCACTTGCTCAAGGTCAGGCAGCTTAGGAAGGGGCAGAT
CGGGGGCTTGAACCCAGGTGGTCAGGCTCTGGAGCCCACAATTGTCTTACCCACT
ATGCCCCTCTCTAGTCATGGTCCCCAAGAGGGGCTTGGAGACCCACTTAGCAGGT
GAAAGCAATGGCAGCCTTCCTTATTTGATTATGCACCTAAGAATAAATGGTATTT
GGGCATGTATTCCCAATATGTGTATATTTATTTATAAATATATACAGATACTATTA
TCTGTATGTTAGTAATAAAGCTTAAATTATTCCATTTTAAAATTATGAATATGAAT
AGGGTTTTTTTTATGTTTCTTGCCTCATCCCAATGACTTTTGCACACCCAGGTGTG
AGCACCCAGCATTCAAGACCACG

SEQ ID NO. 12 is FKRP mRNA transcript variant 1 mRNA, NM_024301.5

CATTGCTCCAAGATGGCGGCGGCGGCGGCAGCGGGAGCGCAGCTCAGCTGGGCT
GGAACTGCCCTCCTGGAACTCCCCCAGCCTACAACCTAGGAGGTGCAGGGACTG
AGGCTCAGGCCAAATCGCAACTCAGACCCAGTGAACCCAAGGCCTGAAGAGAAT
TTGGATTCATTTACCTTGTTTTGTGGGGACTGGAGAGACAAGTAAACTCTCAGAG
TAACTGTCCCCTCTGACTACCATTTCTAAGGATGCCCCGGAGGCCCAGCTAGCCC
CAGACTTCGGCCCCATGCGGCTCACCCGCTGCCAGGCTGCCCTGGCGGCCGCCAT
CACCCTCAACCTTCTGGTCCTCTTCTATGTCTCGTGGCTGCAGCACCAGCCTAGG
AATTCCCGGGCCCGGGGGCCCCGTCGTGCCTCTGCTGCCGGCCCCCGTGTCACCG
TCCTGGTGCGGGAGTTCGAGGCATTTGACAACGCGGTGCCCGAGCTGGTAGACT
CCTTCCTGCAGCAAGACCCAGCCCAGCCCGTGGTGGTGGCAGCCGACACGCTCC
CCTACCCGCCCCTGGCCCTGCCCCGCATCCCCAACGTGCGTCTGGCGCTGCTCCA
GCCCGCCCTGGACCGGCCAGCCGCAGCCTCGCGCCCGGAGACCTACGTGGCCAC
CGAGTTTGTGGCCCTAGTACCTGATGGGGCGCGGGCTGAGGCACCTGGCCTGCTG
GAGCGCATGGTGGAGGCGCTCCGCGCAGGAAGCGCACGTCTGGTGGCCGCCCCG
GTTGCCACGGCCAACCCTGCCAGGTGCCTGGCCCTGAACGTCAGCCTGCGAGAG
TGGACCGCCCGCTATGGCGCAGCCCCCGCCGCGCCCCGCTGCGACGCCCTGGAC
GGAGATGCTGTGGTGCTCCTGCGCGCCCGCGACCTCTTCAACCTCTCGGCGCCCC
TGGCCCGGCCGGTGGGCACCAGCCTCTTTCTGCAGACCGCCCTTCGCGGCTGGGC
GGTGCAGCTGCTGGACTTGACCTTCGCCGCGGCGCGCCAGCCCCCGCTGGCCACG
GCCCACGCGCGCTGGAAGGCTGAGCGCGAGGGACGCGCTCGGCGGGCGGCGCTG
CTCCGCGCGCTGGGCATCCGCCTAGTGAGCTGGGAAGGCGGGCGGCTGGAGTGG
TTCGGCTGCAACAAGGAGACCACGCGCTGCTTCGGAACCGTGGTGGGCGACACG
CCCGCCTACCTCTACGAGGAGCGCTGGACGCCCCCCTGCTGCCTGCGCGCGCTGC
GCGAGACCGCCCGCTATGTGGTGGGCGTGCTGGAGGCTGCGGGCGTGCGCTACT
GGCTCGAGGGCGGCTCACTGCTGGGGGCCGCCCGCCACGGGGACATCATCCCAT
GGGACTACGACGTGGACCTGGGCATCTACTTGGAGGACGTGGGCAACTGCGAGC
AGCTGCGGGGGGCAGAGGCCGGCTCGGTGGTGGATGAGCGCGGCTTCGTATGGG
AGAAGGCGGTCGAGGGCGACTTTTTCCGCGTGCAGTACAGCGAAAGCAACCACT
TGCACGTGGACCTGTGGCCCTTCTACCCCCGCAATGGCGTCATGACCAAGGACAC
GTGGCTGGACCACCGGCAGGATGTGGAGTTTCCCGAGCACTTCCTGCAGCCGCTG
GTGCCCCTGCCCTTTGCCGGCTTCGTGGCGCAGGCGCCTAACAACTACCGCCGCT
TCCTGGAGCTCAAGTTCGGGCCCGGGGTCATCGAGAACCCCCAGTACCCCAACC
CGGCACTGCTGAGTCTGACGGGAAGCGGCTGAAGCCCTGATAACCTCGCCTTTGT
TTTTCGGGGGTCTGTCTGGATGTGGAGAAGCTCTGTGTGAGCGGTGAGGGGTGGA
GGGATGTCGCGGAGAGGGGAAGGGGGAAACTGACCAAGAAAGAAATTCTAAGG
AGAGCATGAGAGAAGGCTGGCATTGGCAGGAGGAGAGCACCAGGACGAGGATG
GGAAGCGACCTCCAGATTTATCAAATGGTCATGCCCACTGGGAGCCGTGGATAT
GCGTGGGGACATCCTGGGTCATCTCAGTCATGGAGGGAGACGGGGATGTCACGC
CGTCCCGCAGGGCCCAGCACAGCCCCAGACCCGAAAAAAGTGTTCTGCCCAAGA
TTCCGAGAGCCCTGCGCTCTAGGGCAGGGGCAGAGTTTTGGAAACAGTGCAGGC
TCTGGAGCCAGACTGGCGAGATTCAAATCCTGGCTCTATCGCTTCGGAGCCAGGT
GGGCCTGGGGGGGCGTCGCAGTCTCTCTGTGCCTCAGTTGCTTCCAGGATGCGGG
ACCCTTGGCTGCAGGGGTTGCTTCCGCCACTAGAGGGCGCGCCGGTCCCGCTCCT
GGTGGCCCACTGTGGCTGCCCGGGCGACAGTACGCCCAGGGCCTGTGTTCCATA
GCCATCTACTCTCTTGAGCCTTTGGACTTCTCTCCAAGCCCCTGTGGGAGGCGGA
CAGCAGTGACCACCTCCCCTTCTTTTGGACTGCGACCTCCTTCCCTCCTGGGAGA
GCCCTGTGACCTGCATGCTACTCTTAACTGTTCTATTCAAGACTGAATAGAAGTA
TTTCAGTCTTGCAGAGGAGGAAATGCTCAGAGCTCCGAGGTGCGGCTGTGGTCG
AGAACCGGGTGCTGGGCCGGGCGCGGGGGCTCACGCCTGTAATCCCAGCACTTT
GGGAGGCCGAGGTGGGAGGATCGCTTGAGCCCAGGAGTCTGAGACCAGCCTCGG
CAACATGCCAAGACCCCGTCTCTATTTTTAAAAAAGAAAAAGAACCGACTTCTG
AATCGCAGCTCCACTCATGACTAATACCTCATTATTTCAGCTGTCTGCACCTAATT
CCCCACTTGCACGGCAGTGTAGACAATAACCATAGCTCACACTCACTGAGCACCT
ACTGGGTACCAGGCACCATTCTCAGTGTTTCACCTGGATCAACTAATGCGTCCCT
CACCTCAGCCCTCTGAAGTGACAGCTGCTATTATTTTCATTACACAGATGAAAAA
GCTGAGGCCAGAATCGTGAAGTCACTTGCTCAAGGTCAGGCAGCTTAGGAAGGG
GCAGATCGGGGGCTTGAACCCAGGTGGTCAGGCTCTGGAGCCCACAATTGTCTT
ACCCACTATGCCCCTCTCTAGTCATGGTCCCCAAGAGGGGCTTGGAGACCCACTT
AGCAGGTGAAAGCAATGGCAGCCTTCCTTATTTGATTATGCACCTAAGAATAAAT
GGTATTTGGGCATGTATTCCCAATATGTGTATATTTATTTATAAATATATACAGAT
ACTATTATCTGTATGTTAGTAATAAAGCTTAAATTATTCCATTTTAAAATTATGAA
TATGAATAGGGTTTTTTTTATGTTTCTTGCCTCATCCCAATGACTTTTGCACACCC
AGGTGTGAGCACCCAGCATTCAAGACCACG

SEQ ID NO. 13 is FKRP, transcript variant X10, mRNA, XM_047439426.1

GGGAAAGGTGGGCTGCTACAAGCAGGAGCGCAGCTCAGCTGGGCTGGAACTGCC
CTCCTGGAACTCCCCCAGCCTACAACCTAGGAGGTGCAGGGACTGAGGCTCAGG
CCAAATCGCAACTCAGACCCAGTGAACCCAAGGCCTGAAGAGAATTTGGATTCA
TTTACCTTGTTTTGTGGGGACTGGAGAGACAAGTAAACTCTCAGAGTAACTGTCC
CCTCTGACTACCATTTCTAAGGCAAGCCCCCTGTTTCTACTCTTGCGCCCCCTGCT
GGTTTCCTGCCCTGTCTGTAAGTTGCATGGCTTTTGTCCGTCTTTTTTTTGTTTGTT
TGTTTGTTTTGAGACAGGGTCTCACCCAGGCTAGAGTGAAGTGGAGCAATCTCGG
CTCACTGCAACCTCCGCCTCCTGAGTTCAAGTGATTCTCACACCTCAGCCTCCCC
AATAGCTGGGATTACAGGATGCCCCGGAGGCCCAGCTAGCCCCAGACTTCGGCC
CCATGCGGCTCACCCGCTGCCAGGCTGCCCTGGCGGCCGCCATCACCCTCAACCT
TCTGGTCCTCTTCTATGTCTCGTGGCTGCAGCACCAGCCTAGGAATTCCCGGGCC
CGGGGGCCCCGTCGTGCCTCTGCTGCCGGCCCCCGTGTCACCGTCCTGGTGCGGG
AGTTCGAGGCATTTGACAACGCGGTGCCCGAGCTGGTAGACTCCTTCCTGCAGCA
AGACCCAGCCCAGCCCGTGGTGGTGGCAGCCGACACGCTCCCCTACCCGCCCCT
GGCCCTGCCCCGCATCCCCAACGTGCGTCTGGCGCTGCTCCAGCCCGCCCTGGAC
CGGCCAGCCGCAGCCTCGCGCCCGGAGACCTACGTGGCCACCGAGTTTGTGGCC
CTAGTACCTGATGGGGCGCGGGCTGAGGCACCTGGCCTGCTGGAGCGCATGGTG
GAGGCGCTCCGCGCAGGAAGCGCACGTCTGGTGGCCGCCCCGGTTGCCACGGCC
AACCCTGCCAGGTGCCTGGCCCTGAACGTCAGCCTGCGAGAGTGGACCGCCCGC
TATGGCGCAGCCCCCGCCGCGCCCCGCTGCGACGCCCTGGACGGAGATGCTGTG
GTGCTCCTGCGCGCCCGCGACCTCTTCAACCTCTCGGCGCCCCTGGCCCGGCCGG
TGGGCACCAGCCTCTTTCTGCAGACCGCCCTTCGCGGCTGGGCGGTGCAGCTGCT
GGACTTGACCTTCGCCGCGGCGCGCCAGCCCCCGCTGGCCACGGCCCACGCGCG
CTGGAAGGCTGAGCGCGAGGGACGCGCTCGGCGGGCGGCGCTGCTCCGCGCGCT
GGGCATCCGCCTAGTGAGCTGGGAAGGCGGGCGGCTGGAGTGGTTCGGCTGCAA
CAAGGAGACCACGCGCTGCTTCGGAACCGTGGTGGGCGACACGCCCGCCTACCT
CTACGAGGAGCGCTGGACGCCCCCCTGCTGCCTGCGCGCGCTGCGCGAGACCGC
CCGCTATGTGGTGGGCGTGCTGGAGGCTGCGGGCGTGCGCTACTGGCTCGAGGG
CGGCTCACTGCTGGGGGCCGCCCGCCACGGGGACATCATCCCATGGGACTACGA
CGTGGACCTGGGCATCTACTTGGAGGACGTGGGCAACTGCGAGCAGCTGCGGGG
GGCAGAGGCCGGCTCGGTGGTGGATGAGCGCGGCTTCGTATGGGAGAAGGCGGT
CGAGGGCGACTTTTTCCGCGTGCAGTACAGCGAAAGCAACCACTTGCACGTGGA
CCTGTGGCCCTTCTACCCCCGCAATGGCGTCATGACCAAGGACACGTGGCTGGAC
CACCGGCAGGATGTGGAGTTTCCCGAGCACTTCCTGCAGCCGCTGGTGCCCCTGC
CCTTTGCCGGCTTCGTGGCGCAGGCGCCTAACAACTACCGCCGCTTCCTGGAGCT
CAAGTTCGGGCCCGGGGTCATCGAGAACCCCCAGTACCCCAACCCGGCACTGCT
GAGTCTGACGGGAAGCGGCTGAAGCCCTGATAACCTCGCCTTTGTTTTTCGGGGG
TCTGTCTGGATGTGGAGAAGCTCTGTGTGAGCGGTGAGGGGTGGAGGGATGTCG
CGGAGAGGGGAAGGGGGAAACTGACCAAGAAAGAAATTCTAAGGAGAGCATGA
GAGAAGGCTGGCATTGGCAGGAGGAGAGCACCAGGACGAGGATGGGAAGCGAC
CTCCAGATTTATCAAATGGTCATGCCCACTGGGAGCCGTGGATATGCGTGGGGAC
ATCCTGGGTCATCTCAGTCATGGAGGGAGACGGGGATGTCACGCCGTCCCGCAG
GGCCCAGCACAGCCCCAGACCCGAAAAAAGTGTTCTGCCCAAGATTCCGAGAGC
CCTGCGCTCTAGGGCAGGGGCAGAGTTTTGGAAACAGTGCAGGCTCTGGAGCCA
GACTGGCGAGATTCAAATCCTGGCTCTATCGCTTCGGAGCCAGGTGGGCCTGGG
GGGGCGTCGCAGTCTCTCTGTGCCTCAGTTGCTTCCAGGATGCGGGACCCTTGGC
TGCAGGGGTTGCTTCCGCCACTAGAGGGCGCGCCGGTCCCGCTCCTGGTGGCCCA
CTGTGGCTGCCCGGGCGACAGTACGCCCAGGGCCTGTGTTCCATAGCCATCTACT
CTCTTGAGCCTTTGGACTTCTCTCCAAGCCCCTGTGGGAGGCGGACAGCAGTGAC
CACCTCCCCTTCTTTTGGACTGCGACCTCCTTCCCTCCTGGGAGAGCCCTGTGACC
TGCATGCTACTCTTAACTGTTCTATTCAAGACTGAATAGAAGTATTTCAGTCTTGC
AGAGGAGGAAATGCTCAGAGCTCCGAGGTGCGGCTGTGGTCGAGAACCGGGTGC
TGGGCCGGGCGCGGGGGCTCACGCCTGTAATCCCAGCACTTTGGGAGGCCGAGG
TGGGAGGATCGCTTGAGCCCAGGAGTCTGAGACCAGCCTCGGCAACATGCCAAG
ACCCCGTCTCTATTTTTAAAAAAGAAAAAGAACCGACTTCTGAATCGCAGCTCCA
CTCATGACTAATACCTCATTATTTCAGCTGTCTGCACCTAATTCCCCACTTGCACG
GCAGTGTAGACAATAACCATAGCTCACACTCACTGAGCACCTACTGGGTACCAG
GCACCATTCTCAGTGTTTCACCTGGATCAACTAATGCGTCCCTCACCTCAGCCCTC
TGAAGTGACAGCTGCTATTATTTTCATTACACAGATGAAAAAGCTGAGGCCAGA
ATCGTGAAGTCACTTGCTCAAGGTCAGGCAGCTTAGGAAGGGGCAGATCGGGGG
CTTGAACCCAGGTGGTCAGGCTCTGGAGCCCACAATTGTCTTACCCACTATGCCC
CTCTCTAGTCATGGTCCCCAAGAGGGGCTTGGAGACCCACTTAGCAGGTGAAAG
CAATGGCAGCCTTCCTTATTTGATTATGCACCTAAGAATAAATGGTATTTGGGCA
TGTATTCCCAATATGTGTATATTTATTTATAAATATATACAGATACTATTATCTGT
ATGTTAGTAATAAAGCTTAAATTATTCCATTTTAAAATTATGAATATGAATAGGG
TTTTTTTTATGTTTCTTGCCTCATCCCAATGACTTTTGCACACCCAGGTGTGAGCA
CCCAGCATTCAAGACCACG

SEQ ID NO. 14 is FKRP, transcript variant X3, mRNA, XM_047439422.1

CATTGCTCCAAGATGGCGGCGGCGGCGGCAGCGGAGGAGATGCTCTGCTGAGGG
GCAGTTGCTATGGTTACAGGGCCTGGACCTTCCTCCGGAAGGTGAGAAACAGGA
GCGCAGCTCAGCTGGGCTGGAACTGCCCTCCTGGAACTCCCCCAGCCTACAACCT
AGGAGGTGCAGGGACTGAGGCTCAGGCCAAATCGCAACTCAGACCCAGTGAACC
CAAGGCCTGAAGAGAATTTGGATTCATTTACCTTGTTTTGTGGGGACTGGAGAGA
CAAGTAAACTCTCAGAGTAACTGTCCCCTCTGACTACCATTTCTAAGGCAAGCCC
CCTGTTTCTACTCTTGCGCCCCCTGCTGGTTTCCTGCCCTGTCTGTAAGTTGCATG
GCTTTTGTCCGTCTTTTTTTTGTTTGTTTGTTTGTTTTGAGACAGGGTCTCACCCAG
GCTAGAGTGAAGTGGAGCAATCTCGGCTCACTGCAACCTCCGCCTCCTGAGTTCA
AGTGATTCTCACACCTCAGCCTCCCCAATAGCTGGGATTACAGGATGCCCCGGAG
GCCCAGCTAGCCCCAGACTTCGGCCCCATGCGGCTCACCCGCTGCCAGGCTGCCC
TGGCGGCCGCCATCACCCTCAACCTTCTGGTCCTCTTCTATGTCTCGTGGCTGCAG
CACCAGCCTAGGAATTCCCGGGCCCGGGGGCCCCGTCGTGCCTCTGCTGCCGGCC
CCCGTGTCACCGTCCTGGTGCGGGAGTTCGAGGCATTTGACAACGCGGTGCCCGA
GCTGGTAGACTCCTTCCTGCAGCAAGACCCAGCCCAGCCCGTGGTGGTGGCAGC
CGACACGCTCCCCTACCCGCCCCTGGCCCTGCCCCGCATCCCCAACGTGCGTCTG
GCGCTGCTCCAGCCCGCCCTGGACCGGCCAGCCGCAGCCTCGCGCCCGGAGACC
TACGTGGCCACCGAGTTTGTGGCCCTAGTACCTGATGGGGCGCGGGCTGAGGCA
CCTGGCCTGCTGGAGCGCATGGTGGAGGCGCTCCGCGCAGGAAGCGCACGTCTG
GTGGCCGCCCCGGTTGCCACGGCCAACCCTGCCAGGTGCCTGGCCCTGAACGTCA
GCCTGCGAGAGTGGACCGCCCGCTATGGCGCAGCCCCCGCCGCGCCCCGCTGCG
ACGCCCTGGACGGAGATGCTGTGGTGCTCCTGCGCGCCCGCGACCTCTTCAACCT
CTCGGCGCCCCTGGCCCGGCCGGTGGGCACCAGCCTCTTTCTGCAGACCGCCCTT
CGCGGCTGGGCGGTGCAGCTGCTGGACTTGACCTTCGCCGCGGCGCGCCAGCCC
CCGCTGGCCACGGCCCACGCGCGCTGGAAGGCTGAGCGCGAGGGACGCGCTCGG
CGGGCGGCGCTGCTCCGCGCGCTGGGCATCCGCCTAGTGAGCTGGGAAGGCGGG
CGGCTGGAGTGGTTCGGCTGCAACAAGGAGACCACGCGCTGCTTCGGAACCGTG
GTGGGCGACACGCCCGCCTACCTCTACGAGGAGCGCTGGACGCCCCCCTGCTGC
CTGCGCGCGCTGCGCGAGACCGCCCGCTATGTGGTGGGCGTGCTGGAGGCTGCG
GGCGTGCGCTACTGGCTCGAGGGCGGCTCACTGCTGGGGGCCGCCCGCCACGGG
GACATCATCCCATGGGACTACGACGTGGACCTGGGCATCTACTTGGAGGACGTG
GGCAACTGCGAGCAGCTGCGGGGGGCAGAGGCCGGCTCGGTGGTGGATGAGCG
CGGCTTCGTATGGGAGAAGGCGGTCGAGGGCGACTTTTTCCGCGTGCAGTACAG
CGAAAGCAACCACTTGCACGTGGACCTGTGGCCCTTCTACCCCCGCAATGGCGTC
ATGACCAAGGACACGTGGCTGGACCACCGGCAGGATGTGGAGTTTCCCGAGCAC
TTCCTGCAGCCGCTGGTGCCCCTGCCCTTTGCCGGCTTCGTGGCGCAGGCGCCTA
ACAACTACCGCCGCTTCCTGGAGCTCAAGTTCGGGCCCGGGGTCATCGAGAACC
CCCAGTACCCCAACCCGGCACTGCTGAGTCTGACGGGAAGCGGCTGAAGCCCTG
ATAACCTCGCCTTTGTTTTTCGGGGGTCTGTCTGGATGTGGAGAAGCTCTGTGTG
AGCGGTGAGGGGTGGAGGGATGTCGCGGAGAGGGGAAGGGGGAAACTGACCAA
GAAAGAAATTCTAAGGAGAGCATGAGAGAAGGCTGGCATTGGCAGGAGGAGAG
CACCAGGACGAGGATGGGAAGCGACCTCCAGATTTATCAAATGGTCATGCCCAC
TGGGAGCCGTGGATATGCGTGGG
GACATCCTGGGTCATCTCAGTCATGGAGGGAGACGGGGATGTCACGCCGTCCCG
CAGGGCCCAGCACAGCCCCAGACCCGAAAAAAGTGTTCTGCCCAAGATTCCGAG
AGCCCTGCGCTCTAGGGCAGGGGCAGAGTTTTGGAAACAGTGCAGGCTCTGGAG
CCAGACTGGCGAGATTCAAATCCTGGCTCTATCGCTTCGGAGCCAGGTGGGCCTG
GGGGGGCGTCGCAGTCTCTCTGTGCCTCAGTTGCTTCCAGGATGCGGGACCCTTG
GCTGCAGG
GGTTGCTTCCGCCACTAGAGGGCGCGCCGGTCCCGCTCCTGGTGGCCCACTGTGG
CTGCCCGGGCGACAGTACGCCCAGGGCCTGTGTTCCATAGCCATCTACTCTCTTG
AGCCTTTGGACTTCTCTCCAAGCCCCTGTGGGAGGCGGACAGCAGTGACCACCTC
CCCTTCTTTTGGACTGCGACCTCCTTCCCTCCTGGGAGAGCCCTGTGACCTGCATG
CTACTCTTAACTGTTCTATTCAAGACTGAATAGAAGTATTTCAGTCTTGCAGAGG
AGGAAATGCTCAGAGCTCCGAGGTGCGGCTGTGGTCGAGAACCGGGTGCTGGGC
CGGGCGCGGGGGCTCACGCCTGTAATCCCAGCACTTTGGGAGGCCGAGGTGGGA
GGATCGCTTGAGCCCAGGAGTCTGAGACCAGCCTCGGCAACATGCCAAGACCCC
GTCTCTATTTTTAAAAAAGAAAAAGAACCGACTTCTGAATCGCAGCTCCACTCAT
GACTAATACCTCATTATTTCAGCTGTCTGCACCTAATTCCCCACTTGCACGGCAGT
GTAGACAATAACCATAGCTCACACTCACTGAGCACCTACTGGGTACCAGGCACC
ATTCTCAGTGTTTCACCTGGATCAACTAATGCGTCCCTCACCTCAGCCCTCTGAA
GTGACAGCTGCTATTATTTTCATTACACAGATGAAAAAGCTGAGGCCAGAATCGT
GAAGTCACTTGCTCAAGGTCAGGCAGCTTAGGAAGGGGCAGATCGGGGGCTTGA
ACCCAGGTGGTCAGGCTCTGGAGCCCACAATTGTCTTACCCACTATGCCCCTCTC
TAGTCATGGTCCCCAAGAGGGGCTTGGAGACCCACTTAGCAGGTGAAAGCAATG
GCAGCCTTCCTTATTTGATTATGCACCTAAGAAT
AAATGGTATTTGGGCATGTATTCCCAATATGTGTATATTTATTTATAAATATATAC
AGATACTATTATCTGTATGTTAGTAATAAAGCTTAAATTATTCCATTTTAAAATTA
TGAATATGAATAGGGTTTTTTTTATGTTTCTTGCCTCATCCCAATGACTTTTGCAC
ACCCAGGTGTGAGCACCCAGCATTCAAGACCACG

SEQ ID NO. 15 is FKRP, transcript variant X5, mRNA, XM_005259247.3

CATTGCTCCAAGATGGCGGCGGCGGCGGCAGCGGAGGAGATGCTCTGCTGAGGG
GCAGTTGCTATGGTTACAGGGCCTGGACCTTCCTCCGGAAGGTGAGAAACAGGA
GCGCAGCTCAGCTGGGCTGGAACTGCCCTCCTGGAACTCCCCCAGCCTACAACCT
AGGAGGTGCAGGGACTGAGGCTCAGGCCAAATCGCAACTCAGACCCAGTGAACC
CAAGGCCTGAAGAGAATTTGGATTCATTTACCTTGTTTTGTGGGGACTGGAGAGA
CAAGTAAACTCTCAGAGTAACTGTCCCCTCTGACTACCATTTCTAAGGCAAGCCC
CCTGTTTCTACTCTTGCGCCCCCTGCTGGTTTCCTGCCCTGTCTGATGCCCCGGAG
GCCCAGCTAGCCCCAGACTTCGGCCCCATGCGGCTCACCCGCTGCCAGGCTGCCC
TGGCGGCCGCCATCACCCTCAACCTTCTGGTCCTCTTCTATGTCTCGTGGCTGCAG
CACCAGCCTAGGAATTCCCGGGCCCGGGGGCCCCGTCGTGCCTCTGCTGCCGGCC
CCCGTGTCACCGTCCTGGTGCGGGAGTTCGAGGCATTTGACAACGCGGTGCCCGA
GCTGGTAGACTCCTTCCTGCAGCAAGACCCAGCCCAGCCCGTGGTGGTGGCAGC
CGACACGCTCCCCTACCCGCCCCTGGCCCTGCCCCGCATCCCCAACGTGCGTCTG
GCGCTGCTCCAGCCCGCCCTGGACCGGCCAGCCGCAGCCTCGCGCCCGGAGACC
TACGTGGCCACCGAGTTTGTGGCCCTAGTACCTGATGGGGCGCGGGCTGAGGCA
CCTGGCCTGCTGGAGCGCATGGTGGAGGCGCTCCGCGCAGGAAGCGCACGTCTG
GTGGCCGCCCCGGTTGCCACGGCCAACCCTGCCAGGTGCCTGGCCCTGAACGTCA
GCCTGCGAGAGTGGACCGCCCGCTATGGCGCAGCCCCCGCCGCGCCCCGCTGCG
ACGCCCTGGACGGAGATGCTGTGGTGCTCCTGCGCGCCCGCGACCTCTTCAACCT
CTCGGCGCCCCTGGCCCGGCCGGTGGGCACCAGCCTCTTTCTGCAGACCGCCCTT
CGCGGCTGGGCGGTGCAGCTGCTGGACTTGACCTTCGCCGCGGCGCGCCAGCCC
CCGCTGGCCACGGCCCACGCGCGCTGGAAGGCTGAGCGCGAGGGACGCGCTCGG
CGGGCGGCGCTGCTCCGCGCGCTGGGCATCCGCCTAGTGAGCTGGGAAGGCGGG
CGGCTGGAGTGGTTCGGCTGCAACAAGGAGACCACGCGCTGCTTCGGAACCGTG
GTGGGCGACACGCCCGCCTACCTCTACGAGGAGCGCTGGACGCCCCCCTGCTGC
CTGCGCGCGCTGCGCGAGACCGCCCGCTATGTGGTGGGCGTGCTGGAGGCTGCG
GGCGTGCGCTACTGGCTCGAGGGCGGCTCACTGCTGGGGGCCGCCCGCCACGGG
GACATCATCCCATGGGACTACGACGTGGACCTGGGCATCTACTTGGAGGACGTG
GGCAACTGCGAGCAGCTGCGGGGGGCAGAGGCCGGCTCGGTGGTGGATGAGCG
CGGCTTCGTATGGGAGAAGGCGGTCGAGGGCGACTTTTTCCGCGTGCAGTACAG
CGAAAGCAACCACTTGCACGTGGACCTGTGGCCCTTCTACCCCCGCAATGGCGTC
ATGACCAAGGACACGTGGCTGGACCACCGGCAGGATGTGGAGTTTCCCGAGCAC
TTCCTGCAGCCGCTGGTGCCCCTGCCCTTTGCCGGCTTCGTGGCGCAGGCGCCTA
ACAACTACCGCCGCTTCCTGGAGCTCAAGTTCGGGCCCGGGGTCATCGAGAACC
CCCAGTACCCCAACCCGGCACTGCTGAGTCTGACGGGAAGCGGCTGAAGCCCTG
ATAACCTCGCCTTTGTTTTTCGGGGGTCTGTCTGGATGTGGAGAAGCTCTGTGTG
AGCGGTGAGGGGTGGAGGGATGTCGCGGAGAGGGGAAGGGGGAAACTGACCAA
GAAAGAAATTCTAAGGAGAGCATGAGAGAAGGCTGGCATTGGCAGGAGGAGAG
CACCAGGACGAGGATGGGAAGCGACCTCCAGATTTATCAAATGGTCATGCCCAC
TGGGAGCCGTGGATATGCGTGGGGACATCCTGGGTCATCTCAGTCATGGAGGGA
GACGGGGATGTCACGCCGTCCCGCAGGGCCCAGCACAGCCCCAGACCCGAAAAA
AGTGTTCTGCCCAAGATTCCGAGAGCCCTGCGCTCTAGGGCAGGGGCAGAGTTTT
GGAAACAGTGCAGGCTCTGGAGCCAGACTGGCGAGATTCAAATCCTGGCTCTAT
CGCTTCGGAGCCAGGTGGGCCTGGGGGGGCGTCGCAGTCTCTCTGTGCCTCAGTT
GCTTCCAGGATGCGGGACCCTTGGCTGCAGGGGTTGCTTCCGCCACTAGAGGGC
GCGCCGGTCCCGCTCCTGGTGGCCCACTGTGGCTGCCCGGGCGACAGTACGCCCA
GGGCCTGTGTTCCATAGCCATCTACTCTCTTGAGCCTTTGGACTTCTCTCCAAGCC
CCTGTGGGAGGCGGACAGCAGTGACCACCTCCCCTTCTTTTGGACTGCGACCTCC
TTCCCTCCTGGGAGAGCCCTGTGACCTGCATGCTACTCTTAACTGTTCTATTCAAG
ACTGAATAGAAGTATTTCAGTCTTGCAGAGGAGGAAATGCTCAGAGCTCCGAGG
TGCGGCTGTGGTCGAGAACCGGGTGCTGGGCCGGGCGCGGGGGCTCACGCCTGT
AATCCCAGCACTTTGGGAGGCCGAGGTGGGAGGATCGCTTGAGCCCAGGAGTCT
GAGACCAGCCTCGGCAACATGCCAAGACCCCGTCTCTATTTTTAAAAAAGAAAA
AGAACCGACTTCTGAATCGCAGCTCCACTCATGACTAATACCTCATTATTTCAGC
TGTCTGCACCTAATTCCCCACTTGCACGGCAGTGTAGACAATAACCATAGCTCAC
ACTCACTGAGCACCTACTGGGTACCAGGCACCATTCTCAGTGTTTCACCTGGATC
AACTAATGCGTCCCTCACCTCAGCCCTCTGAAGTGACAGCTGCTATTATTTTCATT
ACACAGATGAAAAAGCTGAGGCCAGAATCGTGAAGTCACTTGCTCAAGGTCAGG
CAGCTTAGGAAGGGGCAGATCGGGGGCTTGAACCCAGGTGGTCAGGCTCTGGAG
CCCACAATTGTCTTACCCACTATGCCCCTCTCTAGTCATGGTCCCCAAGAGGGGC
TTGGAGACCCACTTAGCAGGTGAAAGCAATGGCAGCCTTCCTTATTTGATTATGC
ACCTAAGAATAAATGGTATTTGGGCATGTATTCCCAATATGTGTATATTTATTTAT
AAATATATACAGATACTATTATCTGTATGTTAGTAATAAAGCTTAAATTATTCCA
TTTTAAAATTATGAATATGAATAGGGTTTTTTTTATGTTTCTTGCCTCATCCCAAT
GACTTTTGCACACCCAGGTGTGAGCACCCAGCATTCAAGACCACG

SEQ ID NO. 16 is FKRP, transcript variant X4, mRNA, XM_005259248.3

CATTGCTCCAAGATGGCGGCGGCGGCGGCAGCGGAGGAGATGCTCTGCTGAGGG
GCAGTTGCTATGGTTACAGGGCCTGGACCTTCCTCCGGAAGGTGAGAAACAGGA
GCGCAGCTCAGCTGGGCTGGAACTGCCCTCCTGGAACTCCCCCAGCCTACAACCT
AGGAGGTGCAGGGACTGAGGCTCAGGCCAAATCGCAACTCAGACCCAGTGAACC
CAAGGCCTGAAGAGAATTTGGATTCATTTACCTTGTTTTGTGGGGACTGGAGAGA
CAAGTAAACTCTCAGAGTAACTGTCCCCTCTGACTACCATTTCTAAGGATGCCCC
GGAGGCCCAGCTAGCCCCAGACTTCGGCCCCATGCGGCTCACCCGCTGCCAGGC
TGCCCTGGCGGCCGCCATCACCCTCAACCTTCTGGTCCTCTTCTATGTCTCGTGGC
TGCAGCACCAGCCTAGGAATTCCCGGGCCCGGGGGCCCCGTCGTGCCTCTGCTGC
CGGCCCCCGTGTCACCGTCCTGGTGCGGGAGTTCGAGGCATTTGACAACGCGGTG
CCCGAGCTGGTAGACTCCTTCCTGCAGCAAGACCCAGCCCAGCCCGTGGTGGTG
GCAGCCGACACGCTCCCCTACCCGCCCCTGGCCCTGCCCCGCATCCCCAACGTGC
GTCTGGCGCTGCTCCAGCCCGCCCTGGACCGGCCAGCCGCAGCCTCGCGCCCGG
AGACCTACGTGGCCACCGAGTTTGTGGCCCTAGTACCTGATGGGGCGCGGGCTG
AGGCACCTGGCCTGCTGGAGCGCATGGTGGAGGCGCTCCGCGCAGGAAGCGCAC
GTCTGGTGGCCGCCCCGGTTGCCACGGCCAACCCTGCCAGGTGCCTGGCCCTGAA
CGTCAGCCTGCGAGAGTGGACCGCCCGCTATGGCGCAGCCCCCGCCGCGCCCCG
CTGCGACGCCCTGGACGGAGATGCTGTGGTGCTCCTGCGCGCCCGCGACCTCTTC
AACCTCTCGGCGCCCCTGGCCCGGCCGGTGGGCACCAGCCTCTTTCTGCAGACCG
CCCTTCGCGGCTGGGCGGTGCAGCTGCTGGACTTGACCTTCGCCGCGGCGCGCCA
GCCCCCGCTGGCCACGGCCCACGCGCGCTGGAAGGCTGAGCGCGAGGGACGCGC
TCGGCGGGCGGCGCTGCTCCGCGCGCTGGGCATCCGCCTAGTGAGCTGGGAAGG
CGGGCGGCTGGAGTGGTTCGGCTGCAACAAGGAGACCACGCGCTGCTTCGGAAC
CGTGGTGGGCGACACGCCCGCCTACCTCTACGAGGAGCGCTGGACGCCCCCCTG
CTGCCTGCGCGCGCTGCGCGAGACCGCCCGCTATGTGGTGGGCGTGCTGGAGGC
TGCGGGCGTGCGCTACTGGCTCGAGGGCGGCTCACTGCTGGGGGCCGCCCGCCA
CGGGGACATCATCCCATGGGACTACGACGTGGACCTGGGCATCTACTTGGAGGA
CGTGGGCAACTGCGAGCAGCTGCGGGGGGCAGAGGCCGGCTCGGTGGTGGATGA
GCGCGGCTTCGTATGGGAGAAGGCGGTCGAGGGCGACTTTTTCCGCGTGCAGTA
CAGCGAAAGCAACCACTTGCACGTGGACCTGTGGCCCTTCTACCCCCGCAATGGC
GTCATGACCAAGGACACGTGGCTGGACCACCGGCAGGATGTGGAGTTTCCCGAG
CACTTCCTGCAGCCGCTGGTGCCCCTGCCCTTTGCCGGCTTCGTGGCGCAGGCGC
CTAACAACTACCGCCGCTTCCTGGAGCTCAAGTTCGGGCCCGGGGTCATCGAGA
ACCCCCAGTACCCCAACCCGGCACTGCTGAGTCTGACGGGAAGCGGCTGAAGCC
CTGATAACCTCGCCTTTGTTTTTCGGGGGTCTGTCTGGATGTGGAGAAGCTCTGTG
TGAGCGGTGAGGGGTGGAGGGATGTCGCGGAGAGGGGAAGGGGGAAACTGACC
AAGAAAGAAATTCTAAGGAGAGCATGAGAGAAGGCTGGCATTGGCAGGAGGAG
AGCACCAGGACGAGGATGGGAAGCGACCTCCAGATTTATCAAATGGTCATGCCC
ACTGGGAGCCGTGGATATGCGTGGGGACATCCTGGGTCATCTCAGTCATGGAGG
GAGACGGGGATGTCACGCCGTCCCGCAGGGCCCAGCACAGCCCCAGACCCGAAA
AAAGTGTTCTGCCCAAGATTCCGAGAGCCCTGCGCTCTAGGGCAGGGGCAGAGT
TTTGGAAACAGTGCAGGCTCTGGAGCCAGACTGGCGAGATTCAAATCCTGGCTCT
ATCGCTTCGGAGCCAGGTGGGCCTGGGGGGGCGTCGCAGTCTCTCTGTGCCTCAG
TTGCTTCCAGGATGCGGGACCCTTGGCTGCAGGGGTTGCTTCCGCCACTAGAGGG
CGCGCCGGTCCCGCTCCTGGTGGCCCACTGTGGCTGCCCGGGCGACAGTACGCCC
AGGGCCTGTGTTCCATAGCCATCTACTCTCTTGAGCCTTTGGACTTCTCTCCAAGC
CCCTGTGGGAGGCGGACAGCAGTGACCACCTCCCCTTCTTTTGGACTGCGACCTC
CTTCCCTCCTGGGAGAGCCCTGTGACCTGCATGCTACTCTTAACTGTTCTATTCAA
GACTGAATAGAAGTATTTCAGTCTTGCAGAGGAGGAAATGCTCAGAGCTCCGAG
GTGCGGCTGTGGTCGAGAACCGGGTGCTGGGCCGGGCGCGGGGGCTCACGCCTG
TAATCCCAGCACTTTGGGAGGCCGAGGTGGGAGGATCGCTTGAGCCCAGGAGTC
TGAGACCAGCCTCGGCAACATGCCAAGACCCCGTCTCTATTTTTAAAAAAGAAA
AAGAACCGACTTCTGAATCGCAGCTCCACTCATGACTAATACCTCATTATTTCAG
CTGTCTGCACCTAATTCCCCACTTGCACGGCAGTGTAGACAATAACCATAGCTCA
CACTCACTGAGCACCTACTGGGTACCAGGCACCATTCTCAGTGTTTCACCTGGAT
CAACTAATGCGTCCCTCACCTCAGCCCTCTGAAGTGACAGCTGCTATTATTTTCAT
TACACAGATGAAAAAGCTGAGGCCAGAATCGTGAAGTCACTTGCTCAAGGTCAG
GCAGCTTAGGAAGGGGCAGATCGGGGGCTTGAACCCAGGTGGTCAGGCTCTGGA
GCCCACAATTGTCTTACCCACTATGCCCCTCTCTAGTCATGGTCCCCAAGAGGGG
CTTGGAGACCCACTTAGCAGGTGAAAGCAATGGCAGCCTTCCTTATTTGATTATG
CACCTAAGAATAAATGGTATTTGGGCATGTATTCCCAATATGTGTATATTTATTTA
TAAATATATACAGATACTATTATCTGTATGTTAGTAATAAAGCTTAAATTATTCC
ATTTTAAAATTATGAATATGAATAGGGTTTTTTTTATGTTTCTTGCCTCATCCCAA
TGACTTTTGCACACCCAGGTGTGAGCACCCAGCATTCAAGACCACG

SEQ ID NO. 17 is FKRP, transcript variant X13, mRNA XM_024451707.2

CATTGCTCCAAGATGGCGGCGGCGGCGGCAGCGGAGGAGATGCTCTGCTGAGGG
GCAGTTGCTATGGTTACAGGGCCTGGACCTTCCTCCGGAAGGTGAGAAACAGGA
GCGCAGCTCAGCTGGGCTGGAACTGCCCTCCTGGAACTCCCCCAGCCTACAACCT
AGGAGGATGCCCCGGAGGCCCAGCTAGCCCCAGACTTCGGCCCCATGCGGCTCA
CCCGCTGCCAGGCTGCCCTGGCGGCCGCCATCACCCTCAACCTTCTGGTCCTCTT
CTATGTCTCGTGGCTGCAGCACCAGCCTAGGAATTCCCGGGCCCGGGGGCCCCGT
CGTGCCTCTGCTGCCGGCCCCCGTGTCACCGTCCTGGTGCGGGAGTTCGAGGCAT
TTGACAACGCGGTGCCCGAGCTGGTAGACTCCTTCCTGCAGCAAGACCCAGCCC
AGCCCGTGGTGGTGGCAGCCGACACGCTCCCCTACCCGCCCCTGGCCCTGCCCCG
CATCCCCAACGTGCGTCTGGCGCTGCTCCAGCCCGCCCTGGACCGGCCAGCCGCA
GCCTCGCGCCCGGAGACCTACGTGGCCACCGAGTTTGTGGCCCTAGTACCTGATG
GGGCGCGGGCTGAGGCACCTGGCCTGCTGGAGCGCATGGTGGAGGCGCTCCGCG
CAGGAAGCGCACGTCTGGTGGCCGCCCCGGTTGCCACGGCCAACCCTGCCAGGT
GCCTGGCCCTGAACGTCAGCCTGCGAGAGTGGACCGCCCGCTATGGCGCAGCCC
CCGCCGCGCCCCGCTGCGACGCCCTGGACGGAGATGCTGTGGTGCTCCTGCGCGC
CCGCGACCTCTTCAACCTCTCGGCGCCCCTGGCCCGGCCGGTGGGCACCAGCCTC
TTTCTGCAGACCGCCCTTCGCGGCTGGGCGGTGCAGCTGCTGGACTTGACCTTCG
CCGCGGCGCGCCAGCCCCCGCTGGCCACGGCCCACGCGCGCTGGAAGGCTGAGC
GCGAGGGACGCGCTCGGCGGGCGGCGCTGCTCCGCGCGCTGGGCATCCGCCTAG
TGAGCTGGGAAGGCGGGCGGCTGGAGTGGTTCGGCTGCAACAAGGAGACCACGC
GCTGCTTCGGAACCGTGGTGGGCGACACGCCCGCCTACCTCTACGAGGAGCGCT
GGACGCCCCCCTGCTGCCTGCGCGCGCTGCGCGAGACCGCCCGCTATGTGGTGG
GCGTGCTGGAGGCTGCGGGCGTGCGCTACTGGCTCGAGGGCGGCTCACTGCTGG
GGGCCGCCCGCCACGGGGACATCATCCCATGGGACTACGACGTGGACCTGGGCA
TCTACTTGGAGGACGTGGGCAACTGCGAGCAGCTGCGGGGGGCAGAGGCCGGCT
CGGTGGTGGATGAGCGCGGCTTCGTATGGGAGAAGGCGGTCGAGGGCGACTTTT
TCCGCGTGCAGTACAGCGAAAGCAACCACTTGCACGTGGACCTGTGGCCCTTCTA
CCCCCGCAATGGCGTCATGACCAAGGACACGTGGCTGGACCACCGGCAGGATGT
GGAGTTTCCCGAGCACTTCCTGCAGCCGCTGGTGCCCCTGCCCTTTGCCGGCTTC
GTGGCGCAGGCGCCTAACAACTACCGCCGCTTCCTGGAGCTCAAGTTCGGGCCC
GGGGTCATCGAGAACCCCCAGTACCCCAACCCGGCACTGCTGAGTCTGACGGGA
AGCGGCTGAAGCCCTGATAACCTCGCCTTTGTTTTTCGGGGGTCTGTCTGGATGT
GGAGAAGCTCTGTGTGAGCGGTGAGGGGTGGAGGGATGTCGCGGAGAGGGGAA
GGGGGAAACTGACCAAGAAAGAAATTCTAAGGAGAGCATGAGAGAAGGCTGGC
ATTGGCAGGAGGAGAGCACCAGGACGAGGATGGGAAGCGACCTCCAGATTTATC
AAATGGTCATGCCCACTGGGAGCCGTGGATATGCGTGGGGACATCCTGGGTCAT
CTCAGTCATGGAGGGAGACGGGGATGTCACGCCGTCCCGCAGGGCCCAGCACAG
CCCCAGACCCGAAAAAAGTGTTCTGCCCAAGATTCCGAGAGCCCTGCGCTCTAG
GGCAGGGGCAGAGTTTTGGAAACAGTGCAGGCTCTGGAGCCAGACTGGCGAGAT
TCAAATCCTGGCTCTATCGCTTCGGAGCCAGGTGGGCCTGGGGGGGCGTCGCAGT
CTCTCTGTGCCTCAGTTGCTTCCAGGATGCGGGACCCTTGGCTGCAGGGGTTGCT
TCCGCCACTAGAGGGCGCGCCGGTCCCGCTCCTGGTGGCCCACTGTGGCTGCCCG
GGCGACAGTACGCCCAGGGCCTGTGTTCCATAGCCATCTACTCTCTTGAGCCTTT
GGACTTCTCTCCAAGCCCCTGTGGGAGGCGGACAGCAGTGACCACCTCCCCTTCT
TTTGGACTGCGACCTCCTTCCCTCCTGGGAGAGCCCTGTGACCTGCATGCTACTCT
TAACTGTTCTATTCAAGACTGAATAGAAGTATTTCAGTCTTGCAGAGGAGGAAAT
GCTCAGAGCTCCGAGGTGCGGCTGTGGTCGAGAACCGGGTGCTGGGCCGGGCGC
GGGGGCTCACGCCTGTAATCCCAGCACTTTGGGAGGCCGAGGTGGGAGGATCGC
TTGAGCCCAGGAGTCTGAGACCAGCCTCGGCAACATGCCAAGACCCCGTCTCTAT
TTTTAAAAAAGAAAAAGAACCGACTTCTGAATCGCAGCTCCACTCATGACTAAT
ACCTCATTATTTCAGCTGTCTGCACCTAATTCCCCACTTGCACGGCAGTGTAGAC
AATAACCATAGCTCACACTCACTGAGCACCTACTGGGTACCAGGCACCATTCTCA
GTGTTTCACCTGGATCAACTAATGCGTCCCTCACCTCAGCCCTCTGAAGTGACAG
CTGCTATTATTTTCATTACACAGATGAAAAAGCTGAGGCCAGAATCGTGAAGTCA
CTTGCTCAAGGTCAGGCAGCTTAGGAAGGGGCAGATCGGGGGCTTGAACCCAGG
TGGTCAGGCTCTGGAGCCCACAATTGTCTTACCCACTATGCCCCTCTCTAGTCAT
GGTCCCCAAGAGGGGCTTGGAGACCCACTTAGCAGGTGAAAGCAATGGCAGCCT
TCCTTATTTGATTATGCACCTAAGAATAAATGGTATTTGGGCATGTATTCCCAATA
TGTGTATATTTATTTATAAATATATACAGATACTATTATCTGTATGTTAGTAATAA
AGCTTAAATTATTCCATTTTAAAATTATGAATATGAATAGGGTTTTTTTTATGTTT
CTTGCCTCATCCCAATGACTTTTGCACACCCAGGTGTGAGCACCCAGCATTCAAG
ACCACG

SEQ ID NO. 18 is FKRP, transcript variant X7, mRNA, XM_047439423.1

CATTGCTCCAAGATGGCGGCGGCGGCGGCAGCGGAGGAGATGCTCTGCTGAGGG
GCAGTTGCTATGGTTACAGGGCCTGGACCTTCCTCCGGAAGGAGCGCAGCTCAG
CTGGGCTGGAACTGCCCTCCTGGAACTCCCCCAGCCTACAACCTAGGAGGTGCA
GGGACTGAGGCTCAGGCCAAATCGCAACTCAGACCCAGTGAACCCAAGGCCTGA
AGAGAATTTGGATTCATTTACCTTGTTTTGTGGGGACTGGAGAGACAAGTAAACT
CTCAGAGTAACTGTCCCCTCTGACTACCATTTCTAAGGCAAGCCCCCTGTTTCTAC
TCTTGCGCCCCCTGCTGGTTTCCTGCCCTGTCTGATGCCCCGGAGGCCCAGCTAG
CCCCAGACTTCGGCCCCATGCGGCTCACCCGCTGCCAGGCTGCCCTGGCGGCCGC
CATCACCCTCAACCTTCTGGTCCTCTTCTATGTCTCGTGGCTGCAGCACCAGCCTA
GGAATTCCCGGGCCCGGGGGCCCCGTCGTGCCTCTGCTGCCGGCCCCCGTGTCAC
CGTCCTGGTGCGGGAGTTCGAGGCATTTGACAACGCGGTGCCCGAGCTGGTAGA
CTCCTTCCTGCAGCAAGACCCAGCCCAGCCCGTGGTGGTGGCAGCCGACACGCTC
CCCTACCCGCCCCTGGCCCTGCCCCGCATCCCCAACGTGCGTCTGGCGCTGCTCC
AGCCCGCCCTGGACCGGCCAGCCGCAGCCTCGCGCCCGGAGACCTACGTGGCCA
CCGAGTTTGTGGCCCTAGTACCTGATGGGGCGCGGGCTGAGGCACCTGGCCTGCT
GGAGCGCATGGTGGAGGCGCTCCGCGCAGGAAGCGCACGTCTGGTGGCCGCCCC
GGTTGCCACGGCCAACCCTGCCAGGTGCCTGGCCCTGAACGTCAGCCTGCGAGA
GTGGACCGCCCGCTATGGCGCAGCCCCCGCCGCGCCCCGCTGCGACGCCCTGGA
CGGAGATGCTGTGGTGCTCCTGCGCGCCCGCGACCTCTTCAACCTCTCGGCGCCC
CTGGCCCGGCCGGTGGGCACCAGCCTCTTTCTGCAGACCGCCCTTCGCGGCTGGG
CGGTGCAGCTGCTGGACTTGACCTTCGCCGCGGCGCGCCAGCCCCCGCTGGCCAC
GGCCCACGCGCGCTGGAAGGCTGAGCGCGAGGGACGCGCTCGGCGGGCGGCGCT
GCTCCGCGCGCTGGGCATCCGCCTAGTGAGCTGGGAAGGCGGGCGGCTGGAGTG
GTTCGGCTGCAACAAGGAGACCACGCGCTGCTTCGGAACCGTGGTGGGCGACAC
GCCCGCCTACCTCTACGAGGAGCGCTGGACGCCCCCCTGCTGCCTGCGCGCGCTG
CGCGAGACCGCCCGCTATGTGGTGGGCGTGCTGGAGGCTGCGGGCGTGCGCTAC
TGGCTCGAGGGCGGCTCACTGCTGGGGGCCGCCCGCCACGGGGACATCATCCCA
TGGGACTACGACGTGGACCTGGGCATCTACTTGGAGGACGTGGGCAACTGCGAG
CAGCTGCGGGGGGCAGAGGCCGGCTCGGTGGTGGATGAGCGCGGCTTCGTATGG
GAGAAGGCGGTCGAGGGCGACTTTTTCCGCGTGCAGTACAGCGAAAGCAACCAC
TTGCACGTGGACCTGTGGCCCTTCTACCCCCGCAATGGCGTCATGACCAAGGACA
CGTGGCTGGACCACCGGCAGGATGTGGAGTTTCCCGAGCACTTCCTGCAGCCGCT
GGTGCCCCTGCCCTTTGCCGGCTTCGTGGCGCAGGCGCCTAACAACTACCGCCGC
TTCCTGGAGCTCAAGTTCGGGCCCGGGGTCATCGAGAACCCCCAGTACCCCAACC
CGGCACTGCTGAGTCTGACGGGAAGCGGCTGAAGCCCTGATAACCTCGCCTTTGT
TTTTCGGGGGTCTGTCTGGATGTGGAGAAGCTCTGTGTGAGCGGTGAGGGGTGGA
GGGATGTCGCGGAGAGGGGAAGGGGGAAACTGACCAAGAAAGAAATTCTAAGG
AGAGCATGAGAGAAGGCTGGCATTGGCAGGAGGAGAGCACCAGGACGAGGATG
GGAAGCGACCTCCAGATTTATCAAATGGTCATGCCCACTGGGAGCCGTGGATAT
GCGTGGGGACATCCTGGGTCATCTCAGTCATGGAGGGAGACGGGGATGTCACGC
CGTCCCGCAGGGCCCAGCACAGCCCCAGACCCGAAAAAAGTGTTCTGCCCAAGA
TTCCGAGAGCCCTGCGCTCTAGGGCAGGGGCAGAGTTTTGGAAACAGTGCAGGC
TCTGGAGCCAGACTGGCGAGATTCAAATCCTGGCTCTATCGCTTCGGAGCCAGGT
GGGCCTGGGGGGGCGTCGCAGTCTCTCTGTGCCTCAGTTGCTTCCAGGATGCGGG
ACCCTTGGCTGCAGGGGTTGCTTCCGCCACTAGAGGGCGCGCCGGTCCCGCTCCT
GGTGGCCCACTGTGGCTGCCCGGGCGACAGTACGCCCAGGGCCTGTGTTCCATA
GCCATCTACTCTCTTGAGCCTTTGGACTTCTCTCCAAGCCCCTGTGGGAGGCGGA
CAGCAGTGACCACCTCCCCTTCTTTTGGACTGCGACCTCCTTCCCTCCTGGGAGA
GCCCTGTGACCTGCATGCTACTCTTAACTGTTCTATTCAAGACTGAATAGAAGTA
TTTCAGTCTTGCAGAGGAGGAAATGCTCAGAGCTCCGAGGTGCGGCTGTGGTCG
AGAACCGGGTGCTGGGCCGGGCGCGGGGGCTCACGCCTGTAATCCCAGCACTTT
GGGAGGCCGAGGTGGGAGGATCGCTTGAGCCCAGGAGTCTGAGACCAGCCTCGG
CAACATGCCAAGACCCCGTCTCTATTTTTAAAAAAGAAAAAGAACCGACTTCTG
AATCGCAGCTCCACTCATGACTAATACCTCATTATTTCAGCTGTCTGCACCTAATT
CCCCACTTGCACGGCAGTGTAGACAATAACCATAGCTCACACTCACTGAGCACCT
ACTGGGTACCAGGCACCATTCTCAGTGTTTCACCTGGATCAACTAATGCGTCCCT
CACCTCAGCCCTCTGAAGTGACAGCTGCTATTATTTTCATTACACAGATGAAAAA
GCTGAGGCCAGAATCGTGAAGTCACTTGCTCAAGGTCAGGCAGCTTAGGAAGGG
GCAGATCGGGGGCTTGAACCCAGGTGGTCAGGCTCTGGAGCCCACAATTGTCTT
ACCCACTATGCCCCTCTCTAGTCATGGTCCCCAAGAGGGGCTTGGAGACCCACTT
AGCAGGTGAAAGCAATGGCAGCCTTCCTTATTTGATTATGCACCTAAGAATAAAT
GGTATTTGGGCATGTATTCCCAATATGTGTATATTTATTTATAAATATATACAGAT
ACTATTATCTGTATGTTAGTAATAAAGCTTAAATTATTCCATTTTAAAATTATGAA
TATGAATAGGGTTTTTTTTATGTTTCTTGCCTCATCCCAATGACTTTTGCACACCC
AGGTGTGAGCACCCAGCATTCAAGACCACG

SEQ ID NO. 19 is FKRP, transcript variant X6, mRNA XM_005259249.5

CATTGCTCCAAGATGGCGGCGGCGGCGGCAGCGGAGGAGATGCTCTGCTG
AGGGGCAGTTGCTATGGTTACAGGGCCTGGACCTTCCTCCGGAAGGAGCG
CAGCTCAGCTGGGCTGGAACTGCCCTCCTGGAACTCCCCCAGCCTACAAC
CTAGGAGGTGCAGGGACTGAGGCTCAGGCCAAATCGCAACTCAGACCCAG
TGAACCCAAGGCCTGAAGAGAATTTGGATTCATTTACCTTGTTTTGTGGG
GACTGGAGAGACAAGTAAACTCTCAGAGTAACTGTCCCCTCTGACTACCA
TTTCTAAGGATGCCCCGGAGGCCCAGCTAGCCCCAGACTTCGGCCCCATG
CGGCTCACCCGCTGCCAGGCTGCCCTGGCGGCCGCCATCACCCTCAACCT
TCTGGTCCTCTTCTATGTCTCGTGGCTGCAGCACCAGCCTAGGAATTCCC
GGGCCCGGGGGCCCCGTCGTGCCTCTGCTGCCGGCCCCCGTGTCACCGTC
CTGGTGCGGGAGTTCGAGGCATTTGACAACGCGGTGCCCGAGCTGGTAGA
CTCCTTCCTGCAGCAAGACCCAGCCCAGCCCGTGGTGGTGGCAGCCGACA
CGCTCCCCTACCCGCCCCTGGCCCTGCCCCGCATCCCCAACGTGCGTCTG
GCGCTGCTCCAGCCCGCCCTGGACCGGCCAGCCGCAGCCTCGCGCCCGGA
GACCTACGTGGCCACCGAGTTTGTGGCCCTAGTACCTGATGGGGCGCGGG
CTGAGGCACCTGGCCTGCTGGAGCGCATGGTGGAGGCGCTCCGCGCAGGA
AGCGCACGTCTGGTGGCCGCCCCGGTTGCCACGGCCAACCCTGCCAGGTG
CCTGGCCCTGAACGTCAGCCTGCGAGAGTGGACCGCCCGCTATGGCGCAG
CCCCCGCCGCGCCCCGCTGCGACGCCCTGGACGGAGATGCTGTGGTGCTC
CTGCGCGCCCGCGACCTCTTCAACCTCTCGGCGCCCCTGGCCCGGCCGGT
GGGCACCAGCCTCTTTCTGCAGACCGCCCTTCGCGGCTGGGCGGTGCAGC
TGCTGGACTTGACCTTCGCCGCGGCGCGCCAGCCCCCGCTGGCCACGGCC
CACGCGCGCTGGAAGGCTGAGCGCGAGGGACGCGCTCGGCGGGCGGCGCT
GCTCCGCGCGCTGGGCATCCGCCTAGTGAGCTGGGAAGGCGGGCGGCTGG
AGTGGTTCGGCTGCAACAAGGAGACCACGCGCTGCTTCGGAACCGTGGTG
GGCGACACGCCCGCCTACCTCTACGAGGAGCGCTGGACGCCCCCCTGCTG
CCTGCGCGCGCTGCGCGAGACCGCCCGCTATGTGGTGGGCGTGCTGGAGG
CTGCGGGCGTGCGCTACTGGCTCGAGGGCGGCTCACTGCTGGGGGCCGCC
CGCCACGGGGACATCATCCCATGGGACTACGACGTGGACCTGGGCATCTA
CTTGGAGGACGTGGGCAACTGCGAGCAGCTGCGGGGGGCAGAGGCCGGCT
CGGTGGTGGATGAGCGCGGCTTCGTATGGGAGAAGGCGGTCGAGGGCGAC
TTTTTCCGCGTGCAGTACAGCGAAAGCAACCACTTGCACGTGGACCTGTG
GCCCTTCTACCCCCGCAATGGCGTCATGACCAAGGACACGTGGCTGGACC
ACCGGCAGGATGTGGAGTTTCCCGAGCACTTCCTGCAGCCGCTGGTGCCC
CTGCCCTTTGCCGGCTTCGTGGCGCAGGCGCCTAACAACTACCGCCGCTT
CCTGGAGCTCAAGTTCGGGCCCGGGGTCATCGAGAACCCCCAGTACCCCA
ACCCGGCACTGCTGAGTCTGACGGGAAGCGGCTGAAGCCCTGATAACCTC
GCCTTTGTTTTTCGGGGGTCTGTCTGGATGTGGAGAAGCTCTGTGTGAGC
GGTGAGGGGTGGAGGGATGTCGCGGAGAGGGGAAGGGGGAAACTGACCAA
GAAAGAAATTCTAAGGAGAGCATGAGAGAAGGCTGGCATTGGCAGGAGGA
GAGCACCAGGACGAGGATGGGAAGCGACCTCCAGATTTATCAAATGGTCA
TGCCCACTGGGAGCCGTGGATATGCGTGGGGACATCCTGGGTCATCTCAG
TCATGGAGGGAGACGGGGATGTCACGCCGTCCCGCAGGGCCCAGCACAGC
CCCAGACCCGAAAAAAGTGTTCTGCCCAAGATTCCGAGAGCCCTGCGCTC
TAGGGCAGGGGCAGAGTTTTGGAAACAGTGCAGGCTCTGGAGCCAGACTG
GCGAGATTCAAATCCTGGCTCTATCGCTTCGGAGCCAGGTGGGCCTGGGG
GGGCGTCGCAGTCTCTCTGTGCCTCAGTTGCTTCCAGGATGCGGGACCCT
TGGCTGCAGGGGTTGCTTCCGCCACTAGAGGGCGCGCCGGTCCCGCTCCT
GGTGGCCCACTGTGGCTGCCCGGGCGACAGTACGCCCAGGGCCTGTGTTC
CATAGCCATCTACTCTCTTGAGCCTTTGGACTTCTCTCCAAGCCCCTGTG
GGAGGCGGACAGCAGTGACCACCTCCCCTTCTTTTGGACTGCGACCTCCT
TCCCTCCTGGGAGAGCCCTGTGACCTGCATGCTACTCTTAACTGTTCTAT
TCAAGACTGAATAGAAGTATTTCAGTCTTGCAGAGGAGGAAATGCTCAGA
GCTCCGAGGTGCGGCTGTGGTCGAGAACCGGGTGCTGGGCCGGGCGCGGG
GGCTCACGCCTGTAATCCCAGCACTTTGGGAGGCCGAGGTGGGAGGATCG
CTTGAGCCCAGGAGTCTGAGACCAGCCTCGGCAACATGCCAAGACCCCGT
CTCTATTTTTAAAAAAGAAAAAGAACCGACTTCTGAATCGCAGCTCCACT
CATGACTAATACCTCATTATTTCAGCTGTCTGCACCTAATTCCCCACTTG
CACGGCAGTGTAGACAATAACCATAGCTCACACTCACTGAGCACCTACTG
GGTACCAGGCACCATTCTCAGTGTTTCACCTGGATCAACTAATGCGTCCC
TCACCTCAGCCCTCTGAAGTGACAGCTGCTATTATTTTCATTACACAGAT
GAAAAAGCTGAGGCCAGAATCGTGAAGTCACTTGCTCAAGGTCAGGCAGC
TTAGGAAGGGGCAGATCGGGGGCTTGAACCCAGGTGGTCAGGCTCTGGAG
CCCACAATTGTCTTACCCACTATGCCCCTCTCTAGTCATGGTCCCCAAGA
GGGGCTTGGAGACCCACTTAGCAGGTGAAAGCAATGGCAGCCTTCCTTAT
TTGATTATGCACCTAAGAATAAATGGTATTTGGGCATGTATTCCCAATAT
GTGTATATTTATTTATAAATATATACAGATACTATTATCTGTATGTTAGT
AATAAAGCTTAAATTATTCCATTTTAAAATTATGAATATGAATAGGGTTT
TTTTTATGTTTCTTGCCTCATCCCAATGACTTTTGCACACCCAGGTGTGA
GCACCCAGCATTCAAGACCACG

SEQ ID NO. 20 is FKRP, transcript variant X15, mRNA, XM_011527307.2

CATTGCTCCAAGATGGCGGCGGCGGCGGCAGCGGAGGAGATGCTCTGCTG
AGGGGCAGTTGCTATGGTTACAGGGCCTGGACCTTCCTCCGGAAGGAGCG
CAGCTCAGCTGGGCTGGAACTGCCCTCCTGGAACTCCCCCAGCCTACAAC
CTAGGAGGATGCCCCGGAGGCCCAGCTAGCCCCAGACTTCGGCCCCATGC
GGCTCACCCGCTGCCAGGCTGCCCTGGCGGCCGCCATCACCCTCAACCTT
CTGGTCCTCTTCTATGTCTCGTGGCTGCAGCACCAGCCTAGGAATTCCCG
GGCCCGGGGGCCCCGTCGTGCCTCTGCTGCCGGCCCCCGTGTCACCGTCC
TGGTGCGGGAGTTCGAGGCATTTGACAACGCGGTGCCCGAGCTGGTAGAC
TCCTTCCTGCAGCAAGACCCAGCCCAGCCCGTGGTGGTGGCAGCCGACAC
GCTCCCCTACCCGCCCCTGGCCCTGCCCCGCATCCCCAACGTGCGTCTGG
CGCTGCTCCAGCCCGCCCTGGACCGGCCAGCCGCAGCCTCGCGCCCGGAG
ACCTACGTGGCCACCGAGTTTGTGGCCCTAGTACCTGATGGGGCGCGGGC
TGAGGCACCTGGCCTGCTGGAGCGCATGGTGGAGGCGCTCCGCGCAGGAA
GCGCACGTCTGGTGGCCGCCCCGGTTGCCACGGCCAACCCTGCCAGGTGC
CTGGCCCTGAACGTCAGCCTGCGAGAGTGGACCGCCCGCTATGGCGCAGC
CCCCGCCGCGCCCCGCTGCGACGCCCTGGACGGAGATGCTGTGGTGCTCC
TGCGCGCCCGCGACCTCTTCAACCTCTCGGCGCCCCTGGCCCGGCCGGTG
GGCACCAGCCTCTTTCTGCAGACCGCCCTTCGCGGCTGGGCGGTGCAGCT
GCTGGACTTGACCTTCGCCGCGGCGCGCCAGCCCCCGCTGGCCACGGCCC
ACGCGCGCTGGAAGGCTGAGCGCGAGGGACGCGCTCGGCGGGCGGCGCTG
CTCCGCGCGCTGGGCATCCGCCTAGTGAGCTGGGAAGGCGGGCGGCTGGA
GTGGTTCGGCTGCAACAAGGAGACCACGCGCTGCTTCGGAACCGTGGTGG
GCGACACGCCCGCCTACCTCTACGAGGAGCGCTGGACGCCCCCCTGCTGC
CTGCGCGCGCTGCGCGAGACCGCCCGCTATGTGGTGGGCGTGCTGGAGGC
TGCGGGCGTGCGCTACTGGCTCGAGGGCGGCTCACTGCTGGGGGCCGCCC
GCCACGGGGACATCATCCCATGGGACTACGACGTGGACCTGGGCATCTAC
TTGGAGGACGTGGGCAACTGCGAGCAGCTGCGGGGGGCAGAGGCCGGCTC
GGTGGTGGATGAGCGCGGCTTCGTATGGGAGAAGGCGGTCGAGGGCGACT
TTTTCCGCGTGCAGTACAGCGAAAGCAACCACTTGCACGTGGACCTGTGG
CCCTTCTACCCCCGCAATGGCGTCATGACCAAGGACACGTGGCTGGACCA
CCGGCAGGATGTGGAGTTTCCCGAGCACTTCCTGCAGCCGCTGGTGCCCC
TGCCCTTTGCCGGCTTCGTGGCGCAGGCGCCTAACAACTACCGCCGCTTC
CTGGAGCTCAAGTTCGGGCCCGGGGTCATCGAGAACCCCCAGTACCCCAA
CCCGGCACTGCTGAGTCTGACGGGAAGCGGCTGAAGCCCTGATAACCTCG
CCTTTGTTTTTCGGGGGTCTGTCTGGATGTGGAGAAGCTCTGTGTGAGCG
GTGAGGGGTGGAGGGATGTCGCGGAGAGGGGAAGGGGGAAACTGACCAAG
AAAGAAATTCTAAGGAGAGCATGAGAGAAGGCTGGCATTGGCAGGAGGAG
AGCACCAGGACGAGGATGGGAAGCGACCTCCAGATTTATCAAATGGTCAT
GCCCACTGGGAGCCGTGGATATGCGTGGGGACATCCTGGGTCATCTCAGT
CATGGAGGGAGACGGGGATGTCACGCCGTCCCGCAGGGCCCAGCACAGCC
CCAGACCCGAAAAAAGTGTTCTGCCCAAGATTCCGAGAGCCCTGCGCTCT
AGGGCAGGGGCAGAGTTTTGGAAACAGTGCAGGCTCTGGAGCCAGACTGG
CGAGATTCAAATCCTGGCTCTATCGCTTCGGAGCCAGGTGGGCCTGGGGG
GGCGTCGCAGTCTCTCTGTGCCTCAGTTGCTTCCAGGATGCGGGACCCTT
GGCTGCAGGGGTTGCTTCCGCCACTAGAGGGCGCGCCGGTCCCGCTCCTG
GTGGCCCACTGTGGCTGCCCGGGCGACAGTACGCCCAGGGCCTGTGTTCC
ATAGCCATCTACTCTCTTGAGCCTTTGGACTTCTCTCCAAGCCCCTGTGG
GAGGCGGACAGCAGTGACCACCTCCCCTTCTTTTGGACTGCGACCTCCTT
CCCTCCTGGGAGAGCCCTGTGACCTGCATGCTACTCTTAACTGTTCTATT
CAAGACTGAATAGAAGTATTTCAGTCTTGCAGAGGAGGAAATGCTCAGAG
CTCCGAGGTGCGGCTGTGGTCGAGAACCGGGTGCTGGGCCGGGCGCGGGG
GCTCACGCCTGTAATCCCAGCACTTTGGGAGGCCGAGGTGGGAGGATCGC
TTGAGCCCAGGAGTCTGAGACCAGCCTCGGCAACATGCCAAGACCCCGTC
TCTATTTTTAAAAAAGAAAAAGAACCGACTTCTGAATCGCAGCTCCACTC
ATGACTAATACCTCATTATTTCAGCTGTCTGCACCTAATTCCCCACTTGC
ACGGCAGTGTAGACAATAACCATAGCTCACACTCACTGAGCACCTACTGG
GTACCAGGCACCATTCTCAGTGTTTCACCTGGATCAACTAATGCGTCCCT
CACCTCAGCCCTCTGAAGTGACAGCTGCTATTATTTTCATTACACAGATG
AAAAAGCTGAGGCCAGAATCGTGAAGTCACTTGCTCAAGGTCAGGCAGCT
TAGGAAGGGGCAGATCGGGGGCTTGAACCCAGGTGGTCAGGCTCTGGAGC
CCACAATTGTCTTACCCACTATGCCCCTCTCTAGTCATGGTCCCCAAGAG
GGGCTTGGAGACCCACTTAGCAGGTGAAAGCAATGGCAGCCTTCCTTATT
TGATTATGCACCTAAGAATAAATGGTATTTGGGCATGTATTCCCAATATG
TGTATATTTATTTATAAATATATACAGATACTATTATCTGTATGTTAGTA
ATAAAGCTTAAATTATTCCATTTTAAAATTATGAATATGAATAGGGTTTT
TTTTATGTTTCTTGCCTCATCCCAATGACTTTTGCACACCCAGGTGTGAG
CACCCAGCATTCAAGACCACG

SEQ ID NO. 21 is FKRP, transcript variant X1, mRNA XM_017027297.3

CATTGCTCCAAGATGGCGGCGGCGGCGGCAGCGGGAGCGCAGCTCAGCTG
GGCTGGAACTGCCCTCCTGGAACTCCCCCAGCCTACAACCTAGGAGGTGC
AGGGACTGAGGCTCAGGCCAAATCGCAACTCAGACCCAGTGAACCCAAGG
CCTGAAGAGAATTTGGATTCATTTACCTTGTTTTGTGGGGACTGGAGAGA
CAAGTAAACTCTCAGAGTAACTGTCCCCTCTGACTACCATTTCTAAGGCA
AGCCCCCTGTTTCTACTCTTGCGCCCCCTGCTGGTTTCCTGCCCTGTCTG
TAAGTTGCATGGCTTTTGTCCGTCTTTTTTTTGTTTGTTTGTTTGTTTTG
AGACAGGGTCTCACCCAGGCTAGAGTGAAGTGGAGCAATCTCGGCTCACT
GCAACCTCCGCCTCCTGAGTTCAAGTGATTCTCACACCTCAGCCTCCCCA
ATAGCTGGGATTACAGGATGCCCCGGAGGCCCAGCTAGCCCCAGACTTCG
GCCCCATGCGGCTCACCCGCTGCCAGGCTGCCCTGGCGGCCGCCATCACC
CTCAACCTTCTGGTCCTCTTCTATGTCTCGTGGCTGCAGCACCAGCCTAG
GAATTCCCGGGCCCGGGGGCCCCGTCGTGCCTCTGCTGCCGGCCCCCGTG
TCACCGTCCTGGTGCGGGAGTTCGAGGCATTTGACAACGCGGTGCCCGAG
CTGGTAGACTCCTTCCTGCAGCAAGACCCAGCCCAGCCCGTGGTGGTGGC
AGCCGACACGCTCCCCTACCCGCCCCTGGCCCTGCCCCGCATCCCCAACG
TGCGTCTGGCGCTGCTCCAGCCCGCCCTGGACCGGCCAGCCGCAGCCTCG
CGCCCGGAGACCTACGTGGCCACCGAGTTTGTGGCCCTAGTACCTGATGG
GGCGCGGGCTGAGGCACCTGGCCTGCTGGAGCGCATGGTGGAGGCGCTCC
GCGCAGGAAGCGCACGTCTGGTGGCCGCCCCGGTTGCCACGGCCAACCCT
GCCAGGTGCCTGGCCCTGAACGTCAGCCTGCGAGAGTGGACCGCCCGCTA
TGGCGCAGCCCCCGCCGCGCCCCGCTGCGACGCCCTGGACGGAGATGCTG
TGGTGCTCCTGCGCGCCCGCGACCTCTTCAACCTCTCGGCGCCCCTGGCC
CGGCCGGTGGGCACCAGCCTCTTTCTGCAGACCGCCCTTCGCGGCTGGGC
GGTGCAGCTGCTGGACTTGACCTTCGCCGCGGCGCGCCAGCCCCCGCTGG
CCACGGCCCACGCGCGCTGGAAGGCTGAGCGCGAGGGACGCGCTCGGCGG
GCGGCGCTGCTCCGCGCGCTGGGCATCCGCCTAGTGAGCTGGGAAGGCGG
GCGGCTGGAGTGGTTCGGCTGCAACAAGGAGACCACGCGCTGCTTCGGAA
CCGTGGTGGGCGACACGCCCGCCTACCTCTACGAGGAGCGCTGGACGCCC
CCCTGCTGCCTGCGCGCGCTGCGCGAGACCGCCCGCTATGTGGTGGGCGT
GCTGGAGGCTGCGGGCGTCGCTACTGGCTCGAGGGCGGCTCACTGCTGGG
GGCCGCCCGCCACGGGGACATCATCCCATGGGACTACGACGTGGACCTGG
GCATCTACTTGGAGGACGTGGGCAACTGCGAGCAGCTGCGGGGGGCAGAG
GCCGGCTCGGTGGTGGATGAGCGCGGCTTCGTATGGGAGAAGGCGGTCGA
GGGCGACTTTTTCCGCGTGCAGTACAGCGAAAGCAACCACTTGCACGTGG
ACCTGTGGCCCTTCTACCCCCGCAATGGCGTCATGACCAAGGACACGTGG
CTGGACCACCGGCAGGATGTGGAGTTTCCCGAGCACTTCCTGCAGCCGCT
GGTGCCCCTGCCCTTTGCCGGCTTCGTGGCGCAGGCGCCTAACAACTACC
GCCGCTTCCTGGAGCTCAAGTTCGGGCCCGGGGTCATCGAGAACCCCCAG
TACCCCAACCCGGCACTGCTGAGTCTGACGGGAAGCGGCTGAAGCCCTGA
TAACCTCGCCTTTGTTTTTCGGGGGTCTGTCTGGATGTGGAGAAGCTCTG
TGTGAGCGGTGAGGGGTGGAGGGATGTCGCGGAGAGGGGAAGGGGGAAAC
TGACCAAGAAAGAAATTCTAAGGAGAGCATGAGAGAAGGCTGGCATTGGC
AGGAGGAGAGCACCAGGACGAGGATGGGAAGCGACCTCCAGATTTATCAA
ATGGTCATGCCCACTGGGAGCCGTGGATATGCGTGGGGACATCCTGGGTC
ATCTCAGTCATGGAGGGAGACGGGGATGTCACGCCGTCCCGCAGGGCCCA
GCACAGCCCCAGACCCGAAAAAAGTGTTCTGCCCAAGATTCCGAGAGCCC
TGCGCTCTAGGGCAGGGGCAGAGTTTTGGAAACAGTGCAGGCTCTGGAGC
CAGACTGGCGAGATTCAAATCCTGGCTCTATCGCTTCGGAGCCAGGTGGG
CCTGGGGGGGCGTCGCAGTCTCTCTGTGCCTCAGTTGCTTCCAGGATGCG
GGACCCTTGGCTGCAGGGGTTGCTTCCGCCACTAGAGGGCGCGCCGGTCC
CGCTCCTGGTGGCCCACTGTGGCTGCCCGGGCGACAGTACGCCCAGGGCC
TGTGTTCCATAGCCATCTACTCTCTTGAGCCTTTGGACTTCTCTCCAAGC
CCCTGTGGGAGGCGGACAGCAGTGACCACCTCCCCTTCTTTTGGACTGCG
ACCTCCTTCCCTCCTGGGAGAGCCCTGTGACCTGCATGCTACTCTTAACT
GTTCTATTCAAGACTGAATAGAAGTATTTCAGTCTTGCAGAGGAGGAAAT
GCTCAGAGCTCCGAGGTGCGGCTGTGGTCGAGAACCGGGTGCTGGGCCGG
GCGCGGGGGCTCACGCCTGTAATCCCAGCACTTTGGGAGGCCGAGGTGGG
AGGATCGCTTGAGCCCAGGAGTCTGAGACCAGCCTCGGCAACATGCCAAG
ACCCCGTCTCTATTTTTAAAAAAGAAAAAGAACCGACTTCTGAATCGCAG
CTCCACTCATGACTAATACCTCATTATTTCAGCTGTCTGCACCTAATTCC
CCACTTGCACGGCAGTGTAGACAATAACCATAGCTCACACTCACTGAGCA
CCTACTGGGTACCAGGCACCATTCTCAGTGTTTCACCTGGATCAACTAAT
GCGTCCCTCACCTCAGCCCTCTGAAGTGACAGCTGCTATTATTTTCATTA
CACAGATGAAAAAGCTGAGGCCAGAATCGTGAAGTCACTTGCTCAAGGTC
AGGCAGCTTAGGAAGGGGCAGATCGGGGGCTTGAACCCAGGTGGTCAGGC
TCTGGAGCCCACAATTGTCTTACCCACTATGCCCCTCTCTAGTCATGGTC
CCCAAGAGGGGCTTGGAGACCCACTTAGCAGGTGAAAGCAATGGCAGCCT
TCCTTATTTGATTATGCACCTAAGAATAAATGGTATTTGGGCATGTATTC
CCAATATGTGTATATTTATTTATAAATATATACAGATACTATTATCTGTA
TGTTAGTAATAAAGCTTAAATTATTCCATTTTAAAATTATGAATATGAAT
AGGGTTTTTTTTATGTTTCTTGCCTCATCCCAATGACTTTTGCACACCCA
GGTGTGAGCACCCAGCATTCAAGACCACG

SEQ ID NO. 22 is FKRP, transcript variant X14, mRNA, XM_011527306.3

CATTGCTCCAAGATGGCGGCGGCGGCGGCAGCGGGAGCGCAGCTCAGCTG
GGCTGGAACTGCCCTCCTGGAACTCCCCCAGCCTACAACCTAGGAGGATG
CCCCGGAGGCCCAGCTAGCCCCAGACTTCGGCCCCATGCGGCTCACCCGC
TGCCAGGCTGCCCTGGCGGCCGCCATCACCCTCAACCTTCTGGTCCTCTT
CTATGTCTCGTGGCTGCAGCACCAGCCTAGGAATTCCCGGGCCCGGGGGC
CCCGTCGTGCCTCTGCTGCCGGCCCCCGTGTCACCGTCCTGGTGCGGGAG
TTCGAGGCATTTGACAACGCGGTGCCCGAGCTGGTAGACTCCTTCCTGCA
GCAAGACCCAGCCCAGCCCGTGGTGGTGGCAGCCGACACGCTCCCCTACC
CGCCCCTGGCCCTGCCCCGCATCCCCAACGTGCGTCTGGCGCTGCTCCAG
CCCGCCCTGGACCGGCCAGCCGCAGCCTCGCGCCCGGAGACCTACGTGGC
CACCGAGTTTGTGGCCCTAGTACCTGATGGGGCGCGGGCTGAGGCACCTG
GCCTGCTGGAGCGCATGGTGGAGGCGCTCCGCGCAGGAAGCGCACGTCTG
GTGGCCGCCCCGGTTGCCACGGCCAACCCTGCCAGGTGCCTGGCCCTGAA
CGTCAGCCTGCGAGAGTGGACCGCCCGCTATGGCGCAGCCCCCGCCGCGC
CCCGCTGCGACGCCCTGGACGGAGATGCTGTGGTGCTCCTGCGCGCCCGC
GACCTCTTCAACCTCTCGGCGCCCCTGGCCCGGCCGGTGGGCACCAGCCT
CTTTCTGCAGACCGCCCTTCGCGGCTGGGCGGTGCAGCTGCTGGACTTGA
CCTTCGCCGCGGCGCGCCAGCCCCCGCTGGCCACGGCCCACGCGCGCTGG
AAGGCTGAGCGCGAGGGACGCGCTCGGCGGGCGGCGCTGCTCCGCGCGCT
GGGCATCCGCCTAGTGAGCTGGGAAGGCGGGCGGCTGGAGTGGTTCGGCT
GCAACAAGGAGACCACGCGCTGCTTCGGAACCGTGGTGGGCGACACGCCC
GCCTACCTCTACGAGGAGCGCTGGACGCCCCCCTGCTGCCTGCGCGCGCT
GCGCGAGACCGCCCGCTATGTGGTGGGCGTGCTGGAGGCTGCGGGCGTGC
GCTACTGGCTCGAGGGCGGCTCACTGCTGGGGGCCGCCCGCCACGGGGAC
ATCATCCCATGGGACTACGACGTGGACCTGGGCATCTACTTGGAGGACGT
GGGCAACTGCGAGCAGCTGCGGGGGGCAGAGGCCGGCTCGGTGGTGGATG
AGCGCGGCTTCGTATGGGAGAAGGCGGTCGAGGGCGACTTTTTCCGCGTG
CAGTACAGCGAAAGCAACCACTTGCACGTGGACCTGTGGCCCTTCTACCC
CCGCAATGGCGTCATGACCAAGGACACGTGGCTGGACCACCGGCAGGATG
TGGAGTTTCCCGAGCACTTCCTGCAGCCGCTGGTGCCCCTGCCCTTTGCC
GGCTTCGTGGCGCAGGCGCCTAACAACTACCGCCGCTTCCTGGAGCTCAA
GTTCGGGCCCGGGGTCATCGAGAACCCCCAGTACCCCAACCCGGCACTGC
TGAGTCTGACGGGAAGCGGCTGAAGCCCTGATAACCTCGCCTTTGTTTTT
CGGGGGTCTGTCTGGATGTGGAGAAGCTCTGTGTGAGCGGTGAGGGGTGG
AGGGATGTCGCGGAGAGGGGAAGGGGGAAACTGACCAAGAAAGAAATTCT
AAGGAGAGCATGAGAGAAGGCTGGCATTGGCAGGAGGAGAGCACCAGGAC
GAGGATGGGAAGCGACCTCCAGATTTATCAAATGGTCATGCCCACTGGGA
GCCGTGGATATGCGTGGGGACATCCTGGGTCATCTCAGTCATGGAGGGAG
ACGGGGATGTCACGCCGTCCCGCAGGGCCCAGCACAGCCCCAGACCCGAA
AAAAGTGTTCTGCCCAAGATTCCGAGAGCCCTGCGCTCTAGGGCAGGGGC
AGAGTTTTGGAAACAGTGCAGGCTCTGGAGCCAGACTGGCGAGATTCAAA
TCCTGGCTCTATCGCTTCGGAGCCAGGTGGGCCTGGGGGGGCGTCGCAGT
CTCTCTGTGCCTCAGTTGCTTCCAGGATGCGGGACCCTTGGCTGCAGGGG
TTGCTTCCGCCACTAGAGGGCGCGCCGGTCCCGCTCCTGGTGGCCCACTG
TGGCTGCCCGGGCGACAGTACGCCCAGGGCCTGTGTTCCATAGCCATCTA
CTCTCTTGAGCCTTTGGACTTCTCTCCAAGCCCCTGTGGGAGGCGGACAG
CAGTGACCACCTCCCCTTCTTTTGGACTGCGACCTCCTTCCCTCCTGGGA
GAGCCCTGTGACCTGCATGCTACTCTTAACTGTTCTATTCAAGACTGAAT
AGAAGTATTTCAGTCTTGCAGAGGAGGAAATGCTCAGAGCTCCGAGGTGC
GGCTGTGGTCGAGAACCGGGTGCTGGGCCGGGCGCGGGGGCTCACGCCTG
TAATCCCAGCACTTTGGGAGGCCGAGGTGGGAGGATCGCTTGAGCCCAGG
AGTCTGAGACCAGCCTCGGCAACATGCCAAGACCCCGTCTCTATTTTTAA
AAAAGAAAAAGAACCGACTTCTGAATCGCAGCTCCACTCATGACTAATAC
CTCATTATTTCAGCTGTCTGCACCTAATTCCCCACTTGCACGGCAGTGTA
GACAATAACCATAGCTCACACTCACTGAGCACCTACTGGGTACCAGGCAC
CATTCTCAGTGTTTCACCTGGATCAACTAATGCGTCCCTCACCTCAGCCC
TCTGAAGTGACAGCTGCTATTATTTTCATTACACAGATGAAAAAGCTGAG
GCCAGAATCGTGAAGTCACTTGCTCAAGGTCAGGCAGCTTAGGAAGGGGC
AGATCGGGGGCTTGAACCCAGGTGGTCAGGCTCTGGAGCCCACAATTGTC
TTACCCACTATGCCCCTCTCTAGTCATGGTCCCCAAGAGGGGCTTGGAGA
CCCACTTAGCAGGTGAAAGCAATGGCAGCCTTCCTTATTTGATTATGCAC
CTAAGAATAAATGGTATTTGGGCATGTATTCCCAATATGTGTATATTTAT
TTATAAATATATACAGATACTATTATCTGTATGTTAGTAATAAAGCTTAA
ATTATTCCATTTTAAAATTATGAATATGAATAGGGTTTTTTTTATGTTTC
TTGCCTCATCCCAATGACTTTTGCACACCCAGGTGTGAGCACCCAGCATT
CAAGACCACG

SEQ ID NO. 23 is FKRP, transcript variant X12, mRNA XM_047439428.1

GGGGACAGTAGGAAGGGGGGCCGGCGGCGAGCGCGAGCCTCGCCCACCCG
GGTCTACACCGAAGGGAGCGCAGCTCAGCTGGGCTGGAACTGCCCTCCTG
GAACTCCCCCAGCCTACAACCTAGGAGGTGCAGGGACTGAGGCTCAGGCC
AAATCGCAACTCAGACCCAGTGAACCCAAGGCCTGAAGAGAATTTGGATT
CATTTACCTTGTTTTGTGGGGACTGGAGAGACAAGTAAACTCTCAGAGTA
ACTGTCCCCTCTGACTACCATTTCTAAGGATGCCCCGGAGGCCCAGCTAG
CCCCAGACTTCGGCCCCATGCGGCTCACCCGCTGCCAGGCTGCCCTGGCG
GCCGCCATCACCCTCAACCTTCTGGTCCTCTTCTATGTCTCGTGGCTGCA
GCACCAGCCTAGGAATTCCCGGGCCCGGGGGCCCCGTCGTGCCTCTGCTG
CCGGCCCCCGTGTCACCGTCCTGGTGCGGGAGTTCGAGGCATTTGACAAC
GCGGTGCCCGAGCTGGTAGACTCCTTCCTGCAGCAAGACCCAGCCCAGCC
CGTGGTGGTGGCAGCCGACACGCTCCCCTACCCGCCCCTGGCCCTGCCCC
GCATCCCCAACGTGCGTCTGGCGCTGCTCCAGCCCGCCCTGGACCGGCCA
GCCGCAGCCTCGCGCCCGGAGACCTACGTGGCCACCGAGTTTGTGGCCCT
AGTACCTGATGGGGCGCGGGCTGAGGCACCTGGCCTGCTGGAGCGCATGG
TGGAGGCGCTCCGCGCAGGAAGCGCACGTCTGGTGGCCGCCCCGGTTGCC
ACGGCCAACCCTGCCAGGTGCCTGGCCCTGAACGTCAGCCTGCGAGAGTG
GACCGCCCGCTATGGCGCAGCCCCCGCCGCGCCCCGCTGCGACGCCCTGG
ACGGAGATGCTGTGGTGCTCCTGCGCGCCCGCGACCTCTTCAACCTCTCG
GCGCCCCTGGCCCGGCCGGTGGGCACCAGCCTCTTTCTGCAGACCGCCCT
TCGCGGCTGGGCGGTGCAGCTGCTGGACTTGACCTTCGCCGCGGCGCGCC
AGCCCCCGCTGGCCACGGCCCACGCGCGCTGGAAGGCTGAGCGCGAGGGA
CGCGCTCGGCGGGCGGCGCTGCTCCGCGCGCTGGGCATCCGCCTAGTGAG
CTGGGAAGGCGGGCGGCTGGAGTGGTTCGGCTGCAACAAGGAGACCACGC
GCTGCTTCGGAACCGTGGTGGGCGACACGCCCGCCTACCTCTACGAGGAG
CGCTGGACGCCCCCCTGCTGCCTGCGCGCGCTGCGCGAGACCGCCCGCTA
TGTGGTGGGCGTGCTGGAGGCTGCGGGCGTGCGCTACTGGCTCGAGGGCG
GCTCACTGCTGGGGGCCGCCCGCCACGGGGACATCATCCCATGGGACTAC
GACGTGGACCTGGGCATCTACTTGGAGGACGTGGGCAACTGCGAGCAGCT
GCGGGGGGCAGAGGCCGGCTCGGTGGTGGATGAGCGCGGCTTCGTATGGG
AGAAGGCGGTCGAGGGCGACTTTTTCCGCGTGCAGTACAGCGAAAGCAAC
CACTTGCACGTGGACCTGTGGCCCTTCTACCCCCGCAATGGCGTCATGAC
CAAGGACACGTGGCTGGACCACCGGCAGGATGTGGAGTTTCCCGAGCACT
TCCTGCAGCCGCTGGTGCCCCTGCCCTTTGCCGGCTTCGTGGCGCAGGCG
CCTAACAACTACCGCCGCTTCCTGGAGCTCAAGTTCGGGCCCGGGGTCAT
CGAGAACCCCCAGTACCCCAACCCGGCACTGCTGAGTCTGACGGGAAGCG
GCTGAAGCCCTGATAACCTCGCCTTTGTTTTTCGGGGGTCTGTCTGGATG
TGGAGAAGCTCTGTGTGAGCGGTGAGGGGTGGAGGGATGTCGCGGAGAGG
GGAAGGGGGAAACTGACCAAGAAAGAAATTCTAAGGAGAGCATGAGAGAA
GGCTGGCATTGGCAGGAGGAGAGCACCAGGACGAGGATGGGAAGCGACCT
CCAGATTTATCAAATGGTCATGCCCACTGGGAGCCGTGGATATGCGTGGG
GACATCCTGGGTCATCTCAGTCATGGAGGGAGACGGGGATGTCACGCCGT
CCCGCAGGGCCCAGCACAGCCCCAGACCCGAAAAAAGTGTTCTGCCCAAG
ATTCCGAGAGCCCTGCGCTCTAGGGCAGGGGCAGAGTTTTGGAAACAGTG
CAGGCTCTGGAGCCAGACTGGCGAGATTCAAATCCTGGCTCTATCGCTTC
GGAGCCAGGTGGGCCTGGGGGGGCGTCGCAGTCTCTCTGTGCCTCAGTTG
CTTCCAGGATGCGGGACCCTTGGCTGCAGGGGTTGCTTCCGCCACTAGAG
GGCGCGCCGGTCCCGCTCCTGGTGGCCCACTGTGGCTGCCCGGGCGACAG
TACGCCCAGGGCCTGTGTTCCATAGCCATCTACTCTCTTGAGCCTTTGGA
CTTCTCTCCAAGCCCCTGTGGGAGGCGGACAGCAGTGACCACCTCCCCTT
CTTTTGGACTGCGACCTCCTTCCCTCCTGGGAGAGCCCTGTGACCTGCAT
GCTACTCTTAACTGTTCTATTCAAGACTGAATAGAAGTATTTCAGTCTTG
CAGAGGAGGAAATGCTCAGAGCTCCGAGGTGCGGCTGTGGTCGAGAACCG
GGTGCTGGGCCGGGCGCGGGGGCTCACGCCTGTAATCCCAGCACTTTGGG
AGGCCGAGGTGGGAGGATCGCTTGAGCCCAGGAGTCTGAGACCAGCCTCG
GCAACATGCCAAGACCCCGTCTCTATTTTTAAAAAAGAAAAAGAACCGAC
TTCTGAATCGCAGCTCCACTCATGACTAATACCTCATTATTTCAGCTGTC
TGCACCTAATTCCCCACTTGCACGGCAGTGTAGACAATAACCATAGCTCA
CACTCACTGAGCACCTACTGGGTACCAGGCACCATTCTCAGTGTTTCACC
TGGATCAACTAATGCGTCCCTCACCTCAGCCCTCTGAAGTGACAGCTGCT
ATTATTTTCATTACACAGATGAAAAAGCTGAGGCCAGAATCGTGAAGTCA
CTTGCTCAAGGTCAGGCAGCTTAGGAAGGGGCAGATCGGGGGCTTGAACC
CAGGTGGTCAGGCTCTGGAGCCCACAATTGTCTTACCCACTATGCCCCTC
TCTAGTCATGGTCCCCAAGAGGGGCTTGGAGACCCACTTAGCAGGTGAAA
GCAATGGCAGCCTTCCTTATTTGATTATGCACCTAAGAATAAATGGTATT
TGGGCATGTATTCCCAATATGTGTATATTTATTTATAAATATATACAGAT
ACTATTATCTGTATGTTAGTAATAAAGCTTAAATTATTCCATTTTAAAAT
TATGAATATGAATAGGGTTTTTTTTATGTTTCTTGCCTCATCCCAATGAC
TTTTGCACACCCAGGTGTGAGCACCCAGCATTCAAGACCACG

SEQ ID NO. 24 is FKRP, transcript variant X16, mRNA XM_047439429.1

GGGGCCGGCGGCGAGCGCGAGCCTCGCCCACCCGGGTCTACACCGAAGGG
AGCGCAGCTCAGCTGGGCTGGAACTGCCCTCCTGGAACTCCCCCAGCCTA
CAACCTAGGAGGATGCCCCGGAGGCCCAGCTAGCCCCAGACTTCGGCCCC
ATGCGGCTCACCCGCTGCCAGGCTGCCCTGGCGGCCGCCATCACCCTCAA
CCTTCTGGTCCTCTTCTATGTCTCGTGGCTGCAGCACCAGCCTAGGAATT
CCCGGGCCCGGGGGCCCCGTCGTGCCTCTGCTGCCGGCCCCCGTGTCACC
GTCCTGGTGCGGGAGTTCGAGGCATTTGACAACGCGGTGCCCGAGCTGGT
AGACTCCTTCCTGCAGCAAGACCCAGCCCAGCCCGTGGTGGTGGCAGCCG
ACACGCTCCCCTACCCGCCCCTGGCCCTGCCCCGCATCCCCAACGTGCGT
CTGGCGCTGCTCCAGCCCGCCCTGGACCGGCCAGCCGCAGCCTCGCGCCC
GGAGACCTACGTGGCCACCGAGTTTGTGGCCCTAGTACCTGATGGGGCGC
GGGCTGAGGCACCTGGCCTGCTGGAGCGCATGGTGGAGGCGCTCCGCGCA
GGAAGCGCACGTCTGGTGGCCGCCCCGGTTGCCACGGCCAACCCTGCCAG
GTGCCTGGCCCTGAACGTCAGCCTGCGAGAGTGGACCGCCCGCTATGGCG
CAGCCCCCGCCGCGCCCCGCTGCGACGCCCTGGACGGAGATGCTGTGGTG
CTCCTGCGCGCCCGCGACCTCTTCAACCTCTCGGCGCCCCTGGCCCGGCC
GGTGGGCACCAGCCTCTTTCTGCAGACCGCCCTTCGCGGCTGGGCGGTGC
AGCTGCTGGACTTGACCTTCGCCGCGGCGCGCCAGCCCCCGCTGGCCACG
GCCCACGCGCGCTGGAAGGCTGAGCGCGAGGGACGCGCTCGGCGGGCGGC
GCTGCTCCGCGCGCTGGGCATCCGCCTAGTGAGCTGGGAAGGCGGGCGGC
TGGAGTGGTTCGGCTGCAACAAGGAGACCACGCGCTGCTTCGGAACCGTG
GTGGGCGACACGCCCGCCTACCTCTACGAGGAGCGCTGGACGCCCCCCTG
CTGCCTGCGCGCGCTGCGCGAGACCGCCCGCTATGTGGTGGGCGTGCTGG
AGGCTGCGGGCGTGCGCTACTGGCTCGAGGGCGGCTCACTGCTGGGGGCC
GCCCGCCACGGGGACATCATCCCATGGGACTACGACGTGGACCTGGGCAT
CTACTTGGAGGACGTGGGCAACTGCGAGCAGCTGCGGGGGGCAGAGGCCG
GCTCGGTGGTGGATGAGCGCGGCTTCGTATGGGAGAAGGCGGTCGAGGGC
GACTTTTTCCGCGTGCAGTACAGCGAAAGCAACCACTTGCACGTGGACCT
GTGGCCCTTCTACCCCCGCAATGGCGTCATGACCAAGGACACGTGGCTGG
ACCACCGGCAGGATGTGGAGTTTCCCGAGCACTTCCTGCAGCCGCTGGTG
CCCCTGCCCTTTGCCGGCTTCGTGGCGCAGGCGCCTAACAACTACCGCCG
CTTCCTGGAGCTCAAGTTCGGGCCCGGGGTCATCGAGAACCCCCAGTACC
CCAACCCGGCACTGCTGAGTCTGACGGGAAGCGGCTGAAGCCCTGATAAC
CTCGCCTTTGTTTTTCGGGGGTCTGTCTGGATGTGGAGAAGCTCTGTGTG
AGCGGTGAGGGGTGGAGGGATGTCGCGGAGAGGGGAAGGGGGAAACTGAC
CAAGAAAGAAATTCTAAGGAGAGCATGAGAGAAGGCTGGCATTGGCAGGA
GGAGAGCACCAGGACGAGGATGGGAAGCGACCTCCAGATTTATCAAATGG
TCATGCCCACTGGGAGCCGTGGATATGCGTGGGGACATCCTGGGTCATCT
CAGTCATGGAGGGAGACGGGGATGTCACGCCGTCCCGCAGGGCCCAGCAC
AGCCCCAGACCCGAAAAAAGTGTTCTGCCCAAGATTCCGAGAGCCCTGCG
CTCTAGGGCAGGGGCAGAGTTTTGGAAACAGTGCAGGCTCTGGAGCCAGA
CTGGCGAGATTCAAATCCTGGCTCTATCGCTTCGGAGCCAGGTGGGCCTG
GGGGGGCGTCGCAGTCTCTCTGTGCCTCAGTTGCTTCCAGGATGCGGGAC
CCTTGGCTGCAGGGGTTGCTTCCGCCACTAGAGGGCGCGCCGGTCCCGCT
CCTGGTGGCCCACTGTGGCTGCCCGGGCGACAGTACGCCCAGGGCCTGTG
TTCCATAGCCATCTACTCTCTTGAGCCTTTGGACTTCTCTCCAAGCCCCT
GTGGGAGGCGGACAGCAGTGACCACCTCCCCTTCTTTTGGACTGCGACCT
CCTTCCCTCCTGGGAGAGCCCTGTGACCTGCATGCTACTCTTAACTGTTC
TATTCAAGACTGAATAGAAGTATTTCAGTCTTGCAGAGGAGGAAATGCTC
AGAGCTCCGAGGTGCGGCTGTGGTCGAGAACCGGGTGCTGGGCCGGGCGC
GGGGGCTCACGCCTGTAATCCCAGCACTTTGGGAGGCCGAGGTGGGAGGA
TCGCTTGAGCCCAGGAGTCTGAGACCAGCCTCGGCAACATGCCAAGACCC
CGTCTCTATTTTTAAAAAAGAAAAAGAACCGACTTCTGAATCGCAGCTCC
ACTCATGACTAATACCTCATTATTTCAGCTGTCTGCACCTAATTCCCCAC
TTGCACGGCAGTGTAGACAATAACCATAGCTCACACTCACTGAGCACCTA
CTGGGTACCAGGCACCATTCTCAGTGTTTCACCTGGATCAACTAATGCGT
CCCTCACCTCAGCCCTCTGAAGTGACAGCTGCTATTATTTTCATTACACA
GATGAAAAAGCTGAGGCCAGAATCGTGAAGTCACTTGCTCAAGGTCAGGC
AGCTTAGGAAGGGGCAGATCGGGGGCTTGAACCCAGGTGGTCAGGCTCTG
GAGCCCACAATTGTCTTACCCACTATGCCCCTCTCTAGTCATGGTCCCCA
AGAGGGGCTTGGAGACCCACTTAGCAGGTGAAAGCAATGGCAGCCTTCCT
TATTTGATTATGCACCTAAGAATAAATGGTATTTGGGCATGTATTCCCAA
TATGTGTATATTTATTTATAAATATATACAGATACTATTATCTGTATGTT
AGTAATAAAGCTTAAATTATTCCATTTTAAAATTATGAATATGAATAGGG
TTTTTTTTATGTTTCTTGCCTCATCCCAATGACTTTTGCACACCCAGGTG
TGAGCACCCAGCATTCAAGACCACG

SEQ ID NO. 25 is FKRP, transcript variant X11, mRNA XM_047439427.1

CGCCCACCCGGGTCTACACCGAAGGGAGCGCAGCTCAGCTGGGCTGGAAC
TGCCCTCCTGGAACTCCCCCAGCCTACAACCTAGGAGGTGCAGGGACTGA
GGCTCAGGCCAAATCGCAACTCAGACCCAGTGAACCCAAGGCCTGAAGAG
AATTTGGATTCATTTACCTTGTTTTGTGGGGACTGGAGAGACAAGTAAAC
TCTCAGAGTAACTGTCCCCTCTGACTACCATTTCTAAGGCAAGCCCCCTG
TTTCTACTCTTGCGCCCCCTGCTGGTTTCCTGCCCTGTCTGTAAGTTGCA
TGGCTTTTGTCCGTCTTTTTTTTGTTTGTTTGTTTGTTTTGAGACAGGGT
CTCACCCAGGCTAGAGTGAAGTGGAGCAATCTCGGCTCACTGCAACCTCC
GCCTCCTGAGTTCAAGTGATTCTCACACCTCAGCCTCCCCAATAGCTGGG
ATTACAGGATGCCCCGGAGGCCCAGCTAGCCCCAGACTTCGGCCCCATGC
GGCTCACCCGCTGCCAGGCTGCCCTGGCGGCCGCCATCACCCTCAACCTT
CTGGTCCTCTTCTATGTCTCGTGGCTGCAGCACCAGCCTAGGAATTCCCG
GGCCCGGGGGCCCCGTCGTGCCTCTGCTGCCGGCCCCCGTGTCACCGTCC
TGGTGCGGGAGTTCGAGGCATTTGACAACGCGGTGCCCGAGCTGGTAGAC
TCCTTCCTGCAGCAAGACCCAGCCCAGCCCGTGGTGGTGGCAGCCGACAC
GCTCCCCTACCCGCCCCTGGCCCTGCCCCGCATCCCCAACGTGCGTCTGG
CGCTGCTCCAGCCCGCCCTGGACCGGCCAGCCGCAGCCTCGCGCCCGGAG
ACCTACGTGGCCACCGAGTTTGTGGCCCTAGTACCTGATGGGGCGCGGGC
TGAGGCACCTGGCCTGCTGGAGCGCATGGTGGAGGCGCTCCGCGCAGGAA
GCGCACGTCTGGTGGCCGCCCCGGTTGCCACGGCCAACCCTGCCAGGTGC
CTGGCCCTGAACGTCAGCCTGCGAGAGTGGACCGCCCGCTATGGCGCAGC
CCCCGCCGCGCCCCGCTGCGACGCCCTGGACGGAGATGCTGTGGTGCTCC
TGCGCGCCCGCGACCTCTTCAACCTCTCGGCGCCCCTGGCCCGGCCGGTG
GGCACCAGCCTCTTTCTGCAGACCGCCCTTCGCGGCTGGGCGGTGCAGCT
GCTGGACTTGACCTTCGCCGCGGCGCGCCAGCCCCCGCTGGCCACGGCCC
ACGCGCGCTGGAAGGCTGAGCGCGAGGGACGCGCTCGGCGGGCGGCGCTG
CTCCGCGCGCTGGGCATCCGCCTAGTGAGCTGGGAAGGCGGGCGGCTGGA
GTGGTTCGGCTGCAACAAGGAGACCACGCGCTGCTTCGGAACCGTGGTGG
GCGACACGCCCGCCTACCTCTACGAGGAGCGCTGGACGCCCCCCTGCTGC
CTGCGCGCGCTGCGCGAGACCGCCCGCTATGTGGTGGGCGTGCTGGAGGC
TGCGGGCGTGCGCTACTGGCTCGAGGGCGGCTCACTGCTGGGGGCCGCCC
GCCACGGGGACATCATCCCATGGGACTACGACGTGGACCTGGGCATCTAC
TTGGAGGACGTGGGCAACTGCGAGCAGCTGCGGGGGGCAGAGGCCGGCTC
GGTGGTGGATGAGCGCGGCTTCGTATGGGAGAAGGCGGTCGAGGGCGACT
TTTTCCGCGTGCAGTACAGCGAAAGCAACCACTTGCACGTGGACCTGTGG
CCCTTCTACCCCCGCAATGGCGTCATGACCAAGGACACGTGGCTGGACCA
CCGGCAGGATGTGGAGTTTCCCGAGCACTTCCTGCAGCCGCTGGTGCCCC
TGCCCTTTGCCGGCTTCGTGGCGCAGGCGCCTAACAACTACCGCCGCTTC
CTGGAGCTCAAGTTCGGGCCCGGGGTCATCGAGAACCCCCAGTACCCCAA
CCCGGCACTGCTGAGTCTGACGGGAAGCGGCTGAAGCCCTGATAACCTCG
CCTTTGTTTTTCGGGGGTCTGTCTGGATGTGGAGAAGCTCTGTGTGAGCG
GTGAGGGGTGGAGGGATGTCGCGGAGAGGGGAAGGGGGAAACTGACCAAG
AAAGAAATTCTAAGGAGAGCATGAGAGAAGGCTGGCATTGGCAGGAGGAG
AGCACCAGGACGAGGATGGGAAGCGACCTCCAGATTTATCAAATGGTCAT
GCCCACTGGGAGCCGTGGATATGCGTGGGGACATCCTGGGTCATCTCAGT
CATGGAGGGAGACGGGGATGTCACGCCGTCCCGCAGGGCCCAGCACAGCC
CCAGACCCGAAAAAAGTGTTCTGCCCAAGATTCCGAGAGCCCTGCGCTCT
AGGGCAGGGGCAGAGTTTTGGAAACAGTGCAGGCTCTGGAGCCAGACTGG
CGAGATTCAAATCCTGGCTCTATCGCTTCGGAGCCAGGTGGGCCTGGGGG
GGCGTCGCAGTCTCTCTGTGCCTCAGTTGCTTCCAGGATGCGGGACCCTT
GGCTGCAGGGGTTGCTTCCGCCACTAGAGGGCGCGCCGGTCCCGCTCCTG
GTGGCCCACTGTGGCTGCCCGGGCGACAGTACGCCCAGGGCCTGTGTTCC
ATAGCCATCTACTCTCTTGAGCCTTTGGACTTCTCTCCAAGCCCCTGTGG
GAGGCGGACAGCAGTGACCACCTCCCCTTCTTTTGGACTGCGACCTCCTT
CCCTCCTGGGAGAGCCCTGTGACCTGCATGCTACTCTTAACTGTTCTATT
CAAGACTGAATAGAAGTATTTCAGTCTTGCAGAGGAGGAAATGCTCAGAG
CTCCGAGGTGCGGCTGTGGTCGAGAACCGGGTGCTGGGCCGGGCGCGGGG
GCTCACGCCTGTAATCCCAGCACTTTGGGAGGCCGAGGTGGGAGGATCGC
TTGAGCCCAGGAGTCTGAGACCAGCCTCGGCAACATGCCAAGACCCCGTC
TCTATTTTTAAAAAAGAAAAAGAACCGACTTCTGAATCGCAGCTCCACTC
ATGACTAATACCTCATTATTTCAGCTGTCTGCACCTAATTCCCCACTTGC
ACGGCAGTGTAGACAATAACCATAGCTCACACTCACTGAGCACCTACTGG
GTACCAGGCACCATTCTCAGTGTTTCACCTGGATCAACTAATGCGTCCCT
CACCTCAGCCCTCTGAAGTGACAGCTGCTATTATTTTCATTACACAGATG
AAAAAGCTGAGGCCAGAATCGTGAAGTCACTTGCTCAAGGTCAGGCAGCT
TAGGAAGGGGCAGATCGGGGGCTTGAACCCAGGTGGTCAGGCTCTGGAGC
CCACAATTGTCTTACCCACTATGCCCCTCTCTAGTCATGGTCCCCAAGAG
GGGCTTGGAGACCCACTTAGCAGGTGAAAGCAATGGCAGCCTTCCTTATT
TGATTATGCACCTAAGAATAAATGGTATTTGGGCATGTATTCCCAATATG
TGTATATTTATTTATAAATATATACAGATACTATTATCTGTATGTTAGTA
ATAAAGCTTAAATTATTCCATTTTAAAATTATGAATATGAATAGGGTTTT
TTTTATGTTTCTTGCCTCATCCCAATGACTTTTGCACACCCAGGTGTGAG
CACCCAGCATTCAAGACCACG

SEQ ID NO. 26 is FKRP, transcript variant X8, mRNA XM_047439424.1

GGGGGCCGGCGGGTGGGAGATCGCGCCCCGAGGAGGGCGAGGGAGACCCA
GGCCCCGCGCGACGGTCCCCCTCGCTGGAGTGAGACTGAGGAAGGGGCCG
CTTTCTGCCCCCCTCCTCCCACTTCGCCCCAGAGAAAGAGCCTTACCCCT
TCCACACCCAAACACCACTCTCCTGGACAATTACTTTTACTCTGAGAGAA
AGGGCGACCAGCAGTGGCCCCATCCTCTCTGATTGCCGGCCAGAGGAGAT
GCTCTGCTGAGGGGCAGTTGCTATGGTTACAGGGCCTGGACCTTCCTCCG
GAAGGAGCGCAGCTCAGCTGGGCTGGAACTGCCCTCCTGGAACTCCCCCA
GCCTACAACCTAGGAGGTGCAGGGACTGAGGCTCAGGCCAAATCGCAACT
CAGACCCAGTGAACCCAAGGCCTGAAGAGAATTTGGATTCATTTACCTTG
TTTTGTGGGGACTGGAGAGACAAGTAAACTCTCAGAGTAACTGTCCCCTC
TGACTACCATTTCTAAGGCAAGCCCCCTGTTTCTACTCTTGCGCCCCCTG
CTGGTTTCCTGCCCTGTCTGTAAGTTGCATGGCTTTTGTCCGTCTTTTTT
TTGTTTGTTTGTTTGTTTTGAGACAGGGTCTCACCCAGGCTAGAGTGAAG
TGGAGCAATCTCGGCTCACTGCAACCTCCGCCTCCTGAGTTCAAGTGATT
CTCACACCTCAGCCTCCCCAATAGCTGGGATTACAGGATGCCCCGGAGGC
CCAGCTAGCCCCAGACTTCGGCCCCATGCGGCTCACCCGCTGCCAGGCTG
CCCTGGCGGCCGCCATCACCCTCAACCTTCTGGTCCTCTTCTATGTCTCG
TGGCTGCAGCACCAGCCTAGGAATTCCCGGGCCCGGGGGCCCCGTCGTGC
CTCTGCTGCCGGCCCCCGTGTCACCGTCCTGGTGCGGGAGTTCGAGGCAT
TTGACAACGCGGTGCCCGAGCTGGTAGACTCCTTCCTGCAGCAAGACCCA
GCCCAGCCCGTGGTGGTGGCAGCCGACACGCTCCCCTACCCGCCCCTGGC
CCTGCCCCGCATCCCCAACGTGCGTCTGGCGCTGCTCCAGCCCGCCCTGG
ACCGGCCAGCCGCAGCCTCGCGCCCGGAGACCTACGTGGCCACCGAGTTT
GTGGCCCTAGTACCTGATGGGGCGCGGGCTGAGGCACCTGGCCTGCTGGA
GCGCATGGTGGAGGCGCTCCGCGCAGGAAGCGCACGTCTGGTGGCCGCCC
CGGTTGCCACGGCCAACCCTGCCAGGTGCCTGGCCCTGAACGTCAGCCTG
CGAGAGTGGACCGCCCGCTATGGCGCAGCCCCCGCCGCGCCCCGCTGCGA
CGCCCTGGACGGAGATGCTGTGGTGCTCCTGCGCGCCCGCGACCTCTTCA
ACCTCTCGGCGCCCCTGGCCCGGCCGGTGGGCACCAGCCTCTTTCTGCAG
ACCGCCCTTCGCGGCTGGGCGGTGCAGCTGCTGGACTTGACCTTCGCCGC
GGCGCGCCAGCCCCCGCTGGCCACGGCCCACGCGCGCTGGAAGGCTGAGC
GCGAGGGACGCGCTCGGCGGGCGGCGCTGCTCCGCGCGCTGGGCATCCGC
CTAGTGAGCTGGGAAGGCGGGCGGCTGGAGTGGTTCGGCTGCAACAAGGA
GACCACGCGCTGCTTCGGAACCGTGGTGGGCGACACGCCCGCCTACCTCT
ACGAGGAGCGCTGGACGCCCCCCTGCTGCCTGCGCGCGCTGCGCGAGACC
GCCCGCTATGTGGTGGGCGTGCTGGAGGCTGCGGGCGTGCGCTACTGGCT
CGAGGGCGGCTCACTGCTGGGGGCCGCCCGCCACGGGGACATCATCCCAT
GGGACTACGACGTGGACCTGGGCATCTACTTGGAGGACGTGGGCAACTGC
GAGCAGCTGCGGGGGGCAGAGGCCGGCTCGGTGGTGGATGAGCGCGGCTT
CGTATGGGAGAAGGCGGTCGAGGGCGACTTTTTCCGCGTGCAGTACAGCG
AAAGCAACCACTTGCACGTGGACCTGTGGCCCTTCTACCCCCGCAATGGC
GTCATGACCAAGGACACGTGGCTGGACCACCGGCAGGATGTGGAGTTTCC
CGAGCACTTCCTGCAGCCGCTGGTGCCCCTGCCCTTTGCCGGCTTCGTGG
CGCAGGCGCCTAACAACTACCGCCGCTTCCTGGAGCTCAAGTTCGGGCCC
GGGGTCATCGAGAACCCCCAGTACCCCAACCCGGCACTGCTGAGTCTGAC
GGGAAGCGGCTGAAGCCCTGATAACCTCGCCTTTGTTTTTCGGGGGTCTG
TCTGGATGTGGAGAAGCTCTGTGTGAGCGGTGAGGGGTGGAGGGATGTCG
CGGAGAGGGGAAGGGGGAAACTGACCAAGAAAGAAATTCTAAGGAGAGCA
TGAGAGAAGGCTGGCATTGGCAGGAGGAGAGCACCAGGACGAGGATGGGA
AGCGACCTCCAGATTTATCAAATGGTCATGCCCACTGGGAGCCGTGGATA
TGCGTGGGGACATCCTGGGTCATCTCAGTCATGGAGGGAGACGGGGATGT
CACGCCGTCCCGCAGGGCCCAGCACAGCCCCAGACCCGAAAAAAGTGTTC
TGCCCAAGATTCCGAGAGCCCTGCGCTCTAGGGCAGGGGCAGAGTTTTGG
AAACAGTGCAGGCTCTGGAGCCAGACTGGCGAGATTCAAATCCTGGCTCT
ATCGCTTCGGAGCCAGGTGGGCCTGGGGGGGCGTCGCAGTCTCTCTGTGC
CTCAGTTGCTTCCAGGATGCGGGACCCTTGGCTGCAGGGGTTGCTTCCGC
CACTAGAGGGCGCGCCGGTCCCGCTCCTGGTGGCCCACTGTGGCTGCCCG
GGCGACAGTACGCCCAGGGCCTGTGTTCCATAGCCATCTACTCTCTTGAG
CCTTTGGACTTCTCTCCAAGCCCCTGTGGGAGGCGGACAGCAGTGACCAC
CTCCCCTTCTTTTGGACTGCGACCTCCTTCCCTCCTGGGAGAGCCCTGTG
ACCTGCATGCTACTCTTAACTGTTCTATTCAAGACTGAATAGAAGTATTT
CAGTCTTGCAGAGGAGGAAATGCTCAGAGCTCCGAGGTGCGGCTGTGGTC
GAGAACCGGGTGCTGGGCCGGGCGCGGGGGCTCACGCCTGTAATCCCAGC
ACTTTGGGAGGCCGAGGTGGGAGGATCGCTTGAGCCCAGGAGTCTGAGAC
CAGCCTCGGCAACATGCCAAGACCCCGTCTCTATTTTTAAAAAAGAAAAA
GAACCGACTTCTGAATCGCAGCTCCACTCATGACTAATACCTCATTATTT
CAGCTGTCTGCACCTAATTCCCCACTTGCACGGCAGTGTAGACAATAACC
ATAGCTCACACTCACTGAGCACCTACTGGGTACCAGGCACCATTCTCAGT
GTTTCACCTGGATCAACTAATGCGTCCCTCACCTCAGCCCTCTGAAGTGA
CAGCTGCTATTATTTTCATTACACAGATGAAAAAGCTGAGGCCAGAATCG
TGAAGTCACTTGCTCAAGGTCAGGCAGCTTAGGAAGGGGCAGATCGGGGG
CTTGAACCCAGGTGGTCAGGCTCTGGAGCCCACAATTGTCTTACCCACTA
TGCCCCTCTCTAGTCATGGTCCCCAAGAGGGGCTTGGAGACCCACTTAGC
AGGTGAAAGCAATGGCAGCCTTCCTTATTTGATTATGCACCTAAGAATAA
ATGGTATTTGGGCATGTATTCCCAATATGTGTATATTTATTTATAAATAT
ATACAGATACTATTATCTGTATGTTAGTAATAAAGCTTAAATTATTCCAT
TTTAAAATTATGAATATGAATAGGGTTTTTTTTATGTTTCTTGCCTCATC
CCAATGACTTTTGCACACCCAGGTGTGAGCACCCAGCATTCAAGACCACG

SEQ ID NO. 27 is FKRP, transcript variant X9, mRNA XM_047439425.1

GGGGGCCGGCGGGTGGGAGATCGCGCCCCGAGGAGGGCGAGGGAGACCCA
GGCCCCGCGCGACGGTCCCCCTCGCTGGAGTGAGACTGAGGAAGGGGCCG
CTTTCTGCCCCCCTCCTCCCACTTCGCCCCAGAGAAAGAGCCTTACCCCT
TCCACACCCAAACACCACTCTCCTGGACAATTACTTTTACTCTGAGAGAA
AGGGCGACCAGCAGTGGCCCCATCCTCTCTGATTGCCGGCCAGGAGCGCA
GCTCAGCTGGGCTGGAACTGCCCTCCTGGAACTCCCCCAGCCTACAACCT
AGGAGGTGCAGGGACTGAGGCTCAGGCCAAATCGCAACTCAGACCCAGTG
AACCCAAGGCCTGAAGAGAATTTGGATTCATTTACCTTGTTTTGTGGGGA
CTGGAGAGACAAGTAAACTCTCAGAGTAACTGTCCCCTCTGACTACCATT
TCTAAGGCAAGCCCCCTGTTTCTACTCTTGCGCCCCCTGCTGGTTTCCTG
CCCTGTCTGTAAGTTGCATGGCTTTTGTCCGTCTTTTTTTTGTTTGTTTG
TTTGTTTTGAGACAGGGTCTCACCCAGGCTAGAGTGAAGTGGAGCAATCT
CGGCTCACTGCAACCTCCGCCTCCTGAGTTCAAGTGATTCTCACACCTCA
GCCTCCCCAATAGCTGGGATTACAGGATGCCCCGGAGGCCCAGCTAGCCC
CAGACTTCGGCCCCATGCGGCTCACCCGCTGCCAGGCTGCCCTGGCGGCC
GCCATCACCCTCAACCTTCTGGTCCTCTTCTATGTCTCGTGGCTGCAGCA
CCAGCCTAGGAATTCCCGGGCCCGGGGGCCCCGTCGTGCCTCTGCTGCCG
GCCCCCGTGTCACCGTCCTGGTGCGGGAGTTCGAGGCATTTGACAACGCG
GTGCCCGAGCTGGTAGACTCCTTCCTGCAGCAAGACCCAGCCCAGCCCGT
GGTGGTGGCAGCCGACACGCTCCCCTACCCGCCCCTGGCCCTGCCCCGCA
TCCCCAACGTGCGTCTGGCGCTGCTCCAGCCCGCCCTGGACCGGCCAGCC
GCAGCCTCGCGCCCGGAGACCTACGTGGCCACCGAGTTTGTGGCCCTAGT
ACCTGATGGGGCGCGGGCTGAGGCACCTGGCCTGCTGGAGCGCATGGTGG
AGGCGCTCCGCGCAGGAAGCGCACGTCTGGTGGCCGCCCCGGTTGCCACG
GCCAACCCTGCCAGGTGCCTGGCCCTGAACGTCAGCCTGCGAGAGTGGAC
CGCCCGCTATGGCGCAGCCCCCGCCGCGCCCCGCTGCGACGCCCTGGACG
GAGATGCTGTGGTGCTCCTGCGCGCCCGCGACCTCTTCAACCTCTCGGCG
CCCCTGGCCCGGCCGGTGGGCACCAGCCTCTTTCTGCAGACCGCCCTTCG
CGGCTGGGCGGTGCAGCTGCTGGACTTGACCTTCGCCGCGGCGCGCCAGC
CCCCGCTGGCCACGGCCCACGCGCGCTGGAAGGCTGAGCGCGAGGGACGC
GCTCGGCGGGCGGCGCTGCTCCGCGCGCTGGGCATCCGCCTAGTGAGCTG
GGAAGGCGGGCGGCTGGAGTGGTTCGGCTGCAACAAGGAGACCACGCGCT
GCTTCGGAACCGTGGTGGGCGACACGCCCGCCTACCTCTACGAGGAGCGC
TGGACGCCCCCCTGCTGCCTGCGCGCGCTGCGCGAGACCGCCCGCTATGT
GGTGGGCGTGCTGGAGGCTGCGGGCGTGCGCTACTGGCTCGAGGGCGGCT
CACTGCTGGGGGCCGCCCGCCACGGGGACATCATCCCATGGGACTACGAC
GTGGACCTGGGCATCTACTTGGAGGACGTGGGCAACTGCGAGCAGCTGCG
GGGGGCAGAGGCCGGCTCGGTGGTGGATGAGCGCGGCTTCGTATGGGAGA
AGGCGGTCGAGGGCGACTTTTTCCGCGTGCAGTACAGCGAAAGCAACCAC
TTGCACGTGGACCTGTGGCCCTTCTACCCCCGCAATGGCGTCATGACCAA
GGACACGTGGCTGGACCACCGGCAGGATGTGGAGTTTCCCGAGCACTTCC
TGCAGCCGCTGGTGCCCCTGCCCTTTGCCGGCTTCGTGGCGCAGGCGCCT
AACAACTACCGCCGCTTCCTGGAGCTCAAGTTCGGGCCCGGGGTCATCGA
GAACCCCCAGTACCCCAACCCGGCACTGCTGAGTCTGACGGGAAGCGGCT
GAAGCCCTGATAACCTCGCCTTTGTTTTTCGGGGGTCTGTCTGGATGTGG
AGAAGCTCTGTGTGAGCGGTGAGGGGTGGAGGGATGTCGCGGAGAGGGGA
AGGGGGAAACTGACCAAGAAAGAAATTCTAAGGAGAGCATGAGAGAAGGC
TGGCATTGGCAGGAGGAGAGCACCAGGACGAGGATGGGAAGCGACCTCCA
GATTTATCAAATGGTCATGCCCACTGGGAGCCGTGGATATGCGTGGGGAC
ATCCTGGGTCATCTCAGTCATGGAGGGAGACGGGGATGTCACGCCGTCCC
GCAGGGCCCAGCACAGCCCCAGACCCGAAAAAAGTGTTCTGCCCAAGATT
CCGAGAGCCCTGCGCTCTAGGGCAGGGGCAGAGTTTTGGAAACAGTGCAG
GCTCTGGAGCCAGACTGGCGAGATTCAAATCCTGGCTCTATCGCTTCGGA
GCCAGGTGGGCCTGGGGGGGCGTCGCAGTCTCTCTGTGCCTCAGTTGCTT
CCAGGATGCGGGACCCTTGGCTGCAGGGGTTGCTTCCGCCACTAGAGGGC
GCGCCGGTCCCGCTCCTGGTGGCCCACTGTGGCTGCCCGGGCGACAGTAC
GCCCAGGGCCTGTGTTCCATAGCCATCTACTCTCTTGAGCCTTTGGACTT
CTCTCCAAGCCCCTGTGGGAGGCGGACAGCAGTGACCACCTCCCCTTCTT
TTGGACTGCGACCTCCTTCCCTCCTGGGAGAGCCCTGTGACCTGCATGCT
ACTCTTAACTGTTCTATTCAAGACTGAATAGAAGTATTTCAGTCTTGCAG
AGGAGGAAATGCTCAGAGCTCCGAGGTGCGGCTGTGGTCGAGAACCGGGT
GCTGGGCCGGGCGCGGGGGCTCACGCCTGTAATCCCAGCACTTTGGGAGG
CCGAGGTGGGAGGATCGCTTGAGCCCAGGAGTCTGAGACCAGCCTCGGCA
ACATGCCAAGACCCCGTCTCTATTTTTAAAAAAGAAAAAGAACCGACTTC
TGAATCGCAGCTCCACTCATGACTAATACCTCATTATTTCAGCTGTCTGC
ACCTAATTCCCCACTTGCACGGCAGTGTAGACAATAACCATAGCTCACAC
TCACTGAGCACCTACTGGGTACCAGGCACCATTCTCAGTGTTTCACCTGG
ATCAACTAATGCGTCCCTCACCTCAGCCCTCTGAAGTGACAGCTGCTATT
ATTTTCATTACACAGATGAAAAAGCTGAGGCCAGAATCGTGAAGTCACTT
GCTCAAGGTCAGGCAGCTTAGGAAGGGGCAGATCGGGGGCTTGAACCCAG
GTGGTCAGGCTCTGGAGCCCACAATTGTCTTACCCACTATGCCCCTCTCT
AGTCATGGTCCCCAAGAGGGGCTTGGAGACCCACTTAGCAGGTGAAAGCA
ATGGCAGCCTTCCTTATTTGATTATGCACCTAAGAATAAATGGTATTTGG
GCATGTATTCCCAATATGTGTATATTTATTTATAAATATATACAGATACT
ATTATCTGTATGTTAGTAATAAAGCTTAAATTATTCCATTTTAAAATTAT
GAATATGAATAGGGTTTTTTTTATGTTTCTTGCCTCATCCCAATGACTTT
TGCACACCCAGGTGTGAGCACCCAGCATTCAAGACCACG

SEQ ID NO. 28 is FKRP, transcript variant X2, mRNA XM_047439421.1

TGTCATGGTAACCCTTCCCCAGCGGTGGGGGCGAGGTGGTGGGGAGCAGC
CTTCCCCTCGCTTCCACCTTTGTGGTTATGGAGCCCCAAACCTCTTAGGA
CTCTGCACCAGGAGAAGGTTGCTAAGGTAACCTAAGTGTCCGTGGTGGTC
CTTCTGCTCCTATCCTCATTGATTACTATGGCAACCTCCCCCCCACCTAG
CATTCAGGTAGAAAGGACCCCAGCCCCAAGGACACTGATCTCTAAGAGAA
GCACCTCTTCCCCAGCCCCTCCCCCATAGCCACCTAGTTGGCATTACCAT
GGTAACCGTCTTCCCCTTCCCATCGTGGGAGGCCCATGCACCAAACTAAC
CAGGTGTTTTTATTTAGAGGAGATGCTCTGCTGAGGGGCAGTTGCTATGG
TTACAGGGCCTGGACCTTCCTCCGGAAGGTGAGAAACAGGAGCGCAGCTC
AGCTGGGCTGGAACTGCCCTCCTGGAACTCCCCCAGCCTACAACCTAGGA
GGTGCAGGGACTGAGGCTCAGGCCAAATCGCAACTCAGACCCAGTGAACC
CAAGGCCTGAAGAGAATTTGGATTCATTTACCTTGTTTTGTGGGGACTGG
AGAGACAAGTAAACTCTCAGAGTAACTGTCCCCTCTGACTACCATTTCTA
AGGATGCCCCGGAGGCCCAGCTAGCCCCAGACTTCGGCCCCATGCGGCTC
ACCCGCTGCCAGGCTGCCCTGGCGGCCGCCATCACCCTCAACCTTCTGGT
CCTCTTCTATGTCTCGTGGCTGCAGCACCAGCCTAGGAATTCCCGGGCCC
GGGGGCCCCGTCGTGCCTCTGCTGCCGGCCCCCGTGTCACCGTCCTGGTG
CGGGAGTTCGAGGCATTTGACAACGCGGTGCCCGAGCTGGTAGACTCCTT
CCTGCAGCAAGACCCAGCCCAGCCCGTGGTGGTGGCAGCCGACACGCTCC
CCTACCCGCCCCTGGCCCTGCCCCGCATCCCCAACGTGCGTCTGGCGCTG
CTCCAGCCCGCCCTGGACCGGCCAGCCGCAGCCTCGCGCCCGGAGACCTA
CGTGGCCACCGAGTTTGTGGCCCTAGTACCTGATGGGGCGCGGGCTGAGG
CACCTGGCCTGCTGGAGCGCATGGTGGAGGCGCTCCGCGCAGGAAGCGCA
CGTCTGGTGGCCGCCCCGGTTGCCACGGCCAACCCTGCCAGGTGCCTGGC
CCTGAACGTCAGCCTGCGAGAGTGGACCGCCCGCTATGGCGCAGCCCCCG
CCGCGCCCCGCTGCGACGCCCTGGACGGAGATGCTGTGGTGCTCCTGCGC
GCCCGCGACCTCTTCAACCTCTCGGCGCCCCTGGCCCGGCCGGTGGGCAC
CAGCCTCTTTCTGCAGACCGCCCTTCGCGGCTGGGCGGTGCAGCTGCTGG
ACTTGACCTTCGCCGCGGCGCGCCAGCCCCCGCTGGCCACGGCCCACGCG
CGCTGGAAGGCTGAGCGCGAGGGACGCGCTCGGCGGGCGGCGCTGCTCCG
CGCGCTGGGCATCCGCCTAGTGAGCTGGGAAGGCGGGCGGCTGGAGTGGT
TCGGCTGCAACAAGGAGACCACGCGCTGCTTCGGAACCGTGGTGGGCGAC
ACGCCCGCCTACCTCTACGAGGAGCGCTGGACGCCCCCCTGCTGCCTGCG
CGCGCTGCGCGAGACCGCCCGCTATGTGGTGGGCGTGCTGGAGGCTGCGG
GCGTGCGCTACTGGCTCGAGGGCGGCTCACTGCTGGGGGCCGCCCGCCAC
GGGGACATCATCCCATGGGACTACGACGTGGACCTGGGCATCTACTTGGA
GGACGTGGGCAACTGCGAGCAGCTGCGGGGGGCAGAGGCCGGCTCGGTGG
TGGATGAGCGCGGCTTCGTATGGGAGAAGGCGGTCGAGGGCGACTTTTTC
CGCGTGCAGTACAGCGAAAGCAACCACTTGCACGTGGACCTGTGGCCCTT
CTACCCCCGCAATGGCGTCATGACCAAGGACACGTGGCTGGACCACCGGC
AGGATGTGGAGTTTCCCGAGCACTTCCTGCAGCCGCTGGTGCCCCTGCCC
TTTGCCGGCTTCGTGGCGCAGGCGCCTAACAACTACCGCCGCTTCCTGGA
GCTCAAGTTCGGGCCCGGGGTCATCGAGAACCCCCAGTACCCCAACCCGG
CACTGCTGAGTCTGACGGGAAGCGGCTGAAGCCCTGATAACCTCGCCTTT
GTTTTTCGGGGGTCTGTCTGGATGTGGAGAAGCTCTGTGTGAGCGGTGAG
GGGTGGAGGGATGTCGCGGAGAGGGGAAGGGGGAAACTGACCAAGAAAGA
AATTCTAAGGAGAGCATGAGAGAAGGCTGGCATTGGCAGGAGGAGAGCAC
CAGGACGAGGATGGGAAGCGACCTCCAGATTTATCAAATGGTCATGCCCA
CTGGGAGCCGTGGATATGCGTGGGGACATCCTGGGTCATCTCAGTCATGG
AGGGAGACGGGGATGTCACGCCGTCCCGCAGGGCCCAGCACAGCCCCAGA
CCCGAAAAAAGTGTTCTGCCCAAGATTCCGAGAGCCCTGCGCTCTAGGGC
AGGGGCAGAGTTTTGGAAACAGTGCAGGCTCTGGAGCCAGACTGGCGAGA
TTCAAATCCTGGCTCTATCGCTTCGGAGCCAGGTGGGCCTGGGGGGGCGT
CGCAGTCTCTCTGTGCCTCAGTTGCTTCCAGGATGCGGGACCCTTGGCTG
CAGGGGTTGCTTCCGCCACTAGAGGGCGCGCCGGTCCCGCTCCTGGTGGC
CCACTGTGGCTGCCCGGGCGACAGTACGCCCAGGGCCTGTGTTCCATAGC
CATCTACTCTCTTGAGCCTTTGGACTTCTCTCCAAGCCCCTGTGGGAGGC
GGACAGCAGTGACCACCTCCCCTTCTTTTGGACTGCGACCTCCTTCCCTC
CTGGGAGAGCCCTGTGACCTGCATGCTACTCTTAACTGTTCTATTCAAGA
CTGAATAGAAGTATTTCAGTCTTGCAGAGGAGGAAATGCTCAGAGCTCCG
AGGTGCGGCTGTGGTCGAGAACCGGGTGCTGGGCCGGGCGCGGGGGCTCA
CGCCTGTAATCCCAGCACTTTGGGAGGCCGAGGTGGGAGGATCGCTTGAG
CCCAGGAGTCTGAGACCAGCCTCGGCAACATGCCAAGACCCCGTCTCTAT
TTTTAAAAAAGAAAAAGAACCGACTTCTGAATCGCAGCTCCACTCATGAC
TAATACCTCATTATTTCAGCTGTCTGCACCTAATTCCCCACTTGCACGGC
AGTGTAGACAATAACCATAGCTCACACTCACTGAGCACCTACTGGGTACC
AGGCACCATTCTCAGTGTTTCACCTGGATCAACTAATGCGTCCCTCACCT
CAGCCCTCTGAAGTGACAGCTGCTATTATTTTCATTACACAGATGAAAAA
GCTGAGGCCAGAATCGTGAAGTCACTTGCTCAAGGTCAGGCAGCTTAGGA
AGGGGCAGATCGGGGGCTTGAACCCAGGTGGTCAGGCTCTGGAGCCCACA
ATTGTCTTACCCACTATGCCCCTCTCTAGTCATGGTCCCCAAGAGGGGCT
TGGAGACCCACTTAGCAGGTGAAAGCAATGGCAGCCTTCCTTATTTGATT
ATGCACCTAAGAATAAATGGTATTTGGGCATGTATTCCCAATATGTGTAT
ATTTATTTATAAATATATACAGATACTATTATCTGTATGTTAGTAATAAA
GCTTAAATTATTCCATTTTAAAATTATGAATATGAATAGGGTTTTTTTTA
TGTTTCTTGCCTCATCCCAATGACTTTTGCACACCCAGGTGTGAGCACCC
AGCATTCAAGACCACG

SEQ ID NO. 29 is the pseudoknot sequence in the 5′ UTR of human FKRP:

AGCTCAGCTGGGCTGGAACTGCCCTCCTGGAACTCCCCCAGCCTACAACC
TAGGAGGTGCAGGGACTGAGGCTCAGGCCAAATCGCAACTCAGACCCAGT
GAACCCAAGGCCTGAAGAGAATTTGGATTCATT

SEQ ID NO. 30 is the PCR forward primer:

TTGTTAACATGCGGCTCACCC

SEQ ID NO. 31 is the PCR reverse primer:

TACCGGTTCAACCGCCTGTC

SEQ ID NO. 32 is a fusion with the C terminus of KLH:

APNNYRRFLELKFGPGVIENPQYPNP

SEQ ID NO. 33 is the first 401 bases from a human FKRP cDNA including 5′ UTR which includes a predicted G-quadriplex, a predicted pseudoknot or an internal ribosome entry site, a predicted hairpin, and an encompassed Kozak consensus sequence:

attgctccaagatggcggcggcggcggcagcgggagcgcagctcagctgg
gctggaactgccctcctggaactcccccagcctacaacctaggaggtgca
gggactgaggctcaggccaaatcgcaactcagacccagtgaacccaaggc
ctgaagagaatttggattcatttaccttgttttgtggggactggagagac
aagtaaactctcagagtaactgtcccctctgactaccatttctaaggcaa
gccccctgtttctactcttgcgccccctgctggtttcctgccctgtctga
tgccccggaggcccagctagccccagactteggccccATGCGGCTCACCC
GCTGCCAGGCTGCCCTGGCGGCCGCCATCACCCTCAACCTTCTGGTCCTC
T

1. A nucleic acid expression cassette comprising a transcriptional regulatory region operably linked to a nucleic acid sequence encoding a fukutin-related protein (FKRP) RNA transcript that comprises a modified 5′ and/or 3′ untranslated region (UTR).

2. The nucleic acid expression cassette of claim 1, wherein the modified 5′ untranslated region (UTR) is truncated as compared to the 5′ UTR of wild-type FKRP.

3. The nucleic acid expression cassette of claim 1, wherein the modified 5′ UTR comprises a deletion of at least one nucleotide, a plurality of nucleotides or all of the nucleotides in the 5′ UTR region.

4. The nucleic acid expression cassette of claim 3, wherein the modification of the 5′UTR comprises deletion or disruption of a G-quadruplex, or a hairpin in the 5′ UTR.

5. The nucleic acid expression cassette of claim 4, wherein the modification comprises a modification to the Kozak consensus sequence.

6. The nucleic acid expression cassette of claim 1, wherein the modified 3′ UTR is truncated compared to the 3′ UTR of wild-type FKRP.

7. The nucleic acid expression cassette of claim 5, wherein the modification to the 3′ UTR comprises deletion of at least one nucleotide, a plurality of nucleotides or all of the nucleotides in the 3′ UTR region.

8. The nucleic acid expression cassette of claim 1, wherein the nucleic acid encoding FKRP comprises a modification in each of the 5′ and 3′ UTRs.

9. The nucleic acid expression cassette of any of claims 1-8, wherein the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

10. The nucleic acid expression cassette of any one of claims 1-9, wherein the transcriptional regulatory region comprises a muscle-specific expression cassette (MSEC).

11. The nucleic acid expression cassette of claim 10, wherein the MSEC is selected from the group consisting of CK8e.

12. The nucleic acid expression cassette of claim 11, wherein upon administration to a cell, expression level of an FKRP mRNA or protein is higher when operably linked to an MSEC than the expression level of the FKRP mRNA or protein when operably linked to a CK8e transcriptional regulatory region.

13. The nucleic acid expression cassette of claim 11, wherein upon administration to a cell, expression level of an FKRP mRNA or protein is lower when operably linked to an MSEC than the expression level of the FKRP mRNA or protein when operably linked to a CK8e transcriptional regulatory region.

14. An RNA transcript generated by transcription of the nucleic acid expression cassette of any one of claims 1-13.

15. An adeno-associated viral vector (AAV) comprising the nucleic acid expression cassette of any one of claims 1-13.

16. The AAV vector of claim 15, wherein the adeno-associated viral vector is selected from the group consisting of: an AAVRh74 vector, an AAV8 vector, an AAV9 vector, an AAV6 vector, an AAV7 vector, an AAV218 vector, a NP vector, a NP 66 vector, a NP 22 vector, an AAVpo.1 vector, a MyoAAV vector, and an AAVMyo vector.

17. The AAV vector of claim 15, wherein the adeno-associated viral vector comprises an internal terminal repeat (ITR), a muscle-specific expression cassette, a nucleic acid encoding FKRP, a polyadenylation signal (pA+), and/or a second ITR.

18. An engineered cell comprising or expressing a nucleic acid expression cassette of any one of claims 1-17.

19. The engineered cell of claim 18, wherein the modified 5′ untranslated region (UTR) is truncated as compared to the wild-type 5′ UTR of FKRP.

20. The engineered cell of claim 18 or 19, wherein the modified 5′ UTR comprises a deletion of at least one nucleotide, a plurality of nucleotides or all of the nucleotides in the 5′ UTR region.

21. The engineered cell of claim 20, wherein the modification of the 5′UTR comprises deletion or disruption of a G-quadruplex, or a hairpin in the 5′ UTR.

22. The engineered cell of claim 20 or 21, wherein the modification comprises a modification to the Kozak consensus sequence.

23. The engineered cell of claim 18, wherein the modified 3′ UTR is truncated compared to the 3′UTR of wild-type FKRP.

24. The engineered cell of claim 23, wherein the modification to the 3′ UTR comprises deletion of at least one nucleotide, a plurality of nucleotides or all of the nucleotides in the 3′ UTR region.

25. The engineered cell of claim 18, wherein the nucleic acid encoding FKRP comprises a modification in each of the 5′ and 3′ UTRs.

26. The engineered cell of any of claims 18-25, wherein the modification in the 5′ and/or 3′ UTR of FKRP causes an increase or a decrease in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

27. A method of expressing an FKRP gene product in a subject comprising administering an adeno-associated viral vector of any one of claims 15-17 to a subject in need thereof.

28. The method of claim 27, wherein the FKRP gene product is a RNA transcript and/or a protein.

29. The method of claim 27, wherein the subject in need thereof comprises limb girdle muscular dystrophy type 2I/R9 (LGMD2i), Walker-Warburg syndrome, or muscle-eye-brain disease (MED).

30. The method of claim 27, wherein the modified 5′ untranslated region (UTR) is truncated as compared to the 5′ UTR of wild-type FKRP.

31. The method of claim 27, wherein the modified 5′ UTR comprises a deletion of at least one nucleotide, a plurality of nucleotides or all of the nucleotides in the 5′ UTR region.

32. The method of claim 31, wherein the modification of the 5′UTR comprises deletion or disruption of a G-quadruplex, or a hairpin in the 5′ UTR.

33. The method of claim 32, wherein the modification comprises a modification to the Kozak consensus sequence.

34. The method of claim 27, wherein the modified 3′ UTR is truncated compared to the 3′ UTR of a wild-type FKRP.

35. The method of claim 34, wherein the modification to the 3′ UTR comprises deletion of at least one nucleotide, a plurality of nucleotides or all of the nucleotides in the 3′ UTR region.

36. The method of claim 27, wherein the nucleic acid encoding FKRP comprises a modification in each of the 5′ and 3′ UTRs.

37. The method of any of claims 27-36, wherein the modification in the 5′ and/or 3′ UTR of FKRP causes a reduction or inhibition in protein expression and/or enzymatic activity upon expression in a cell as compared to the protein expression and/or enzymatic activity expressed from a similar construct comprising a transcriptional regulatory region operably linked to a nucleic acid sequence encoding a FKRP RNA transcript that comprises a wild-type 5′ and/or 3′ untranslated region (UTR).

38. The method of claim 27, wherein the administration of the AAV vector comprises intravenous and/or intramuscular injection.

39. The method of claim 27, wherein the subject is a human.

40. A method for reducing at least one symptom of an FKRP-mediated disease or disorder, the method comprising administering an AAV vector of any one of claims 15-17 to a subject in need thereof, thereby reducing at least one symptom of an FKRP-mediated disorder.

41. The method of claim 40, wherein the FKRP-mediated disease or disorder comprises limb girdle muscular dystrophy type 2I/R9 (LGMD2i), Walker-Warburg syndrome, or muscle-eye-brain disease (MED).

42. The method of claim 40, wherein the modified 5′ untranslated region (UTR) is truncated as compared to the wild-type 5′ UTR of FKRP.

43. The method of claim 40, wherein the modified 5′ UTR comprises a deletion of at least one nucleotide, a plurality of nucleotides or all of the nucleotides in the 5′ UTR region.

44. The method of claim 43, wherein the modification of the 5′UTR comprises deletion or disruption of a G-quadruplex, or a hairpin in the 5′ UTR.

45. The method of claim 44, wherein the modification comprises a modification to the Kozak consensus sequence.

46. The method of claim 40, wherein the modified 3′ UTR is truncated compared to the 3′ UTR of a wild-type FKRP.

47. The method of claim 46, wherein the modification to the 3′ UTR comprises deletion of at least one nucleotide, a plurality of nucleotides or all of the nucleotides in the 3′ UTR region.

48. The method of claim 40, wherein the nucleic acid encoding FKRP comprises a modification in each of the 5′ and 3′ UTRs.

49. The method of any of claims 40-48, wherein the modification in the 5′ and/or 3′ UTR of FKRP causes an increase or a decrease in protein expression and/or enzymatic activity upon expression in a cell as compared to the protein expression and/or enzymatic activity of a construct comprising wild type 5′ and 3′ FKRP UTRs under substantially similar conditions.

50. The method of claim 40, wherein the administration of the AAV vector comprises intravenous and/or intramuscular injection.

51. The method of claim 40, wherein the subject is a human.

52. The method of claim 40, wherein at least one symptom of a FKRP-mediated disease or disorder comprises: muscle pain, muscle weakness, muscle fatigue, muscle atrophy, inflammation, decrease in average myofiber diameter in skeletal muscle, loss of ambulation, abnormalities in the brain and/or eyes, eye problems, delay in development, intellectual disability, and seizures.
